(19) United States
(12) Patent Application Publication
(10) Pub. No.: US 2025/0084098 A1
(43) Pub. Date: Mar. 13, 2025

(54) FUSED-RING AMINE DERIVATIVE

(71) Applicants: Sumitomo Pharma Co., Ltd., Osaka (JP); CARNA BIOSCIENCES, INC., Hyogo (JP)

(72) Inventors: Shingo TOJO, Osaka-shi, Osaka (JP); Daisuke URABE, Osaka-shi, Osaka (JP); Hitoshi WATANABE, Osaka-shi, Osaka (JP)

(73) Assignees: Sumitomo Pharma Co., Ltd., Osaka (JP); CARNA BIOSCIENCES, INC., Hyogo (JP)

(21) Appl. No.: 18/292,585

(22) PCT Filed: Jul. 27, 2022
(86) PCT No.: PCT/JP2022/028901

§ 371 Date: Jan. 26, 2024

(30) Foreign Application Priority Data
Jul. 28, 2021 (JP) 2021-123159
Mar. 17, 2022 (JP) 2022-042450

(51) Int. Cl. C07D 513/04; A61K 31/428; A61K 31/5377; A61K 45/06

(52) U.S. Cl. CPC C07D 513/04; A61K 31/428; A61K 31/5377; A61K 45/06

(57) ABSTRACT

The present invention provides a fused-ring amine derivative that has a DYRK inhibitory effect and that is represented by formula (1)

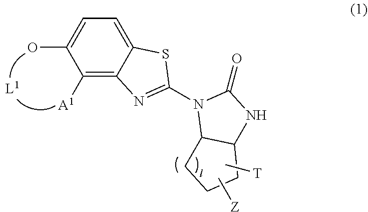

(refer to the description with respect to A¹, L¹, T, Z and l in the formula), or a pharmaceutically acceptable salt thereof.

### TECHNICAL FIELD

The present invention relates to a medicament, particularly a novel fused amine derivative having a DYRK inhibitory effect or a pharmaceutically acceptable salt thereof.

### BACKGROUND ART

DYRK (dual-specificity tyrosine-phosphorylation regulated protein kinase) is one of the bispecific protein kinases that phosphorylate tyrosine, serine, and threonine. DYRK functions as a tyrosine kinase only in the case of autophosphorylation and catalyzes the phosphorylation of serine or threonine residues on exogenous substrates. Five members of the DYRK family are known in humans: DYRK1A, DYRK1B, DYRK2, DYRK3, and DYRK4 (Non Patent Literature 1).

It has been widely reported that DYRK1A is associated with noeropsychiatric diseases. For example, in patients with Alzheimer's disease, the expression of β-amyloid is significantly consistent with that of DYRK1A (Non Patent Literature 2), and it is speculated that DYRK1A is involved in abnormal phosphorylation of a tau protein (Tau), which is considered to contribute to the onset of Alzheimer's disease (Non Patent Literature 3).

In addition, Parkinson's disease is a neurodegenerative disease caused by the degeneration of dopamine neurons, which are important for motor function, but one of the causes is considered to be mitochondrial dysfunction (Non Patent Literarure 4). An enzyme involved in protein degradation called Parkin is known to metabolize abnormal mitochondria and suppress abnormal accumulation, but DYRK1A has been reported to suppress the activity of this parkin protein (Non Patent Literature 5).

The gene for DYRK1A is located in the Down's syndrome critical region, and it has been reported that mice overexpressing DYRK1A exhibit neuropsychiatric dysfunction and appear like Down's syndrome (Non Patent Literature 6). It has also been reported that DYRK1A expression is increased in the brains of patients with Down's syndrome and Down's syndrome-like model mice (Non Patent Literature 7). These reports suggest that DYRK1A is involved in the onset of neurological symptoms in the patients with Down's syndrome (Non Patent Literature 8).

In addition, it has been reported that early-onset Alzheimer's disease occurs frequently in patients with Down's syndrome, thus indicating that DYRK1A is closely related to Alzheimer's disease (Non Patent Literature 8).

Therefore, compounds inhibiting DYRK1A are considered useful for treating neuropsychiatric diseases such as Alzheimer's disease, Down's syndrome, mental retardation, memory impairment, memory loss, and Parkinson's disease.

Recently, it has been reported that DYRK1A is highly expressed in brain tumors such as glioblastoma and regulates the expression of EGFR (Non Patent Literature 9). Therefore, compounds inhibiting DYRK1A are considered useful for treating an epidermal growth factor receptor (EGER)-dependent cancers by suppressing the proliferation of cancer ceils in EGFR-dependent brain tumors and other tumors, or the like that is EGFR dependent.

Compounds inhibiting the family enzymes DYRK1B, DYRK2, and DYRK3 are also considered to have various pharmaceutical applications. For example, it has been reported that DYRK1B is highly expressed in quiescent (G0-phase) cancer cells and contributes to resistance to various chemotherapeutic agents (Non Patent Literature 10). It has also been reported that inhibition of DYRK1B promotes withdrawal from the G0 phase and enhances sensitivity to chemotherapeutic agents (Non Patent Literature 11). Therefore, compounds inhibiting DYRK1B are considered useful for treating pancreatic cancer, ovarian cancer, osteosarcoma, colorectal cancer, and lung cancer (Non Patent Literatures 11, 12, 13, 14, and 15).

It is suggested that DYRK2 controls p53 to induce apoptosis in response to DNA damages (Non Patent Literature 16). Furthermore, it has been reported that compounds inhibiting DYRK3 are useful for treat no sickle cell anemia and chronic kidney disease (Non Patent Literature 17).

In addition to Patent Literature 1 for compounds inhibiting DYRK, Patent Literature 2 has been reported for DYRK1A and DYRK1B inhibitors. However, the amine derivative of the present invention is not disclosed therein.

### PRIOR ART LITERATURE(S)

### Patent Literature (s)

Patent Literature 1: WO2010/10797
Patent Literature 2: WO2013/26806

### Non Patent Literature(s)

Non-Patent Literature 1: Becker W. et al., J. Biol. Chem., 1998, 273, 25893-25902
Non-Patent Literature 2: Kimura R. et al., Hum. Mol. Genet., 2007, 16, 15-23
Non-Patent Literature 3: Ryoo S R. et al., J. Biol. Chem., 2007, 282, 34850-34857
Non-Patent Literature 4: Narendra D. et al., J. Cell. Biol., 2008, 183, 795-803
Non-Patent Literature 5: Im E., J. Neurochem., 2015, 134, 756-768
Non-Patent Literature 6: Branchi I. et al., J. Neuropathol. Exp. Neurol., 2004, 63, 429-440
Non-Patent Literature 7: Dowjat W K. et al., Neurosci. Lett., 2007, 413, 77-81
Non-Patent Literature 8: Wegiel J. et al., FEBS J., 2011, 278, 236-245
Non-Patent Literature 9: Pozo N. et al., J. Clin. Invest., 2013, 123, 2475-2487.
Non-Patent Literature 10: Deng X. et al., Cancer Res., 2006, 66, 4149-4158.
Non-Patent Literature 11: Ewton D Z. et al., Mol. Cancer Ther., 2011, 10, 2104-2114.
Non-Patent Literature 12: Deng X. et al., Genes Cancer., 2014, 5, 201-211.
Non-Patent Literature 13: Yang C. et al., Cacionogenesis., 2010, 31, 552-558.
Non-Patent Literature 14: Jin K. et al., J. Biol. Chem., 2009, 284, 22916-22925.
Non-Patent Literature 15: Gao J et al., Cancer Cell Int. 2013, 13, 2
Non-Patent Literature 16: Taira N. et al., Mol. Cell., 2007, 25, 725-738.
Non-Patent Literature 17: Bogacheva O. et al., J. Biol. Chem., 2008, 283, 36665-36675.

### SUMMARY OF THE INVENTION

### Problem to be Solved by the Invention

An object of the present invention is to provide a medicament, particularly a novel fused amine derivative having a DYRK inhibitory effect or a pharmaceutically acceptable salt thereof.

### Means for Solving the Problem

That is, the present invention is as follows.

(1) A compound represented by:

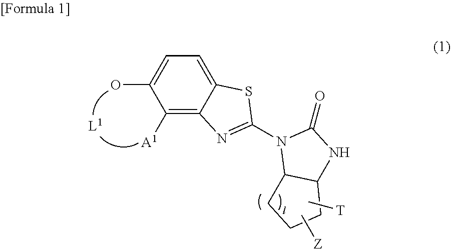

wherein
A¹ represents optionally substituted methylene or an oxygen atom,
L¹ represents optionally substituted methylene or optionally substituted ethylene,
l represents 1, 2, or 3,
T represents a hydrogen atom or optionally substituted C₁₋₆; alkyl,
Z represents —NR¹R² or —OR³,
R¹ and R² each independently represent a hydrogen atom, optionally substituted C₁₋₆ alkyl, or C(O)—R^{A}, or R¹ and R², together with the nitrogen atom to which they are attached, may form an optionally substituted 4- to 7-membered saturated heterocycle,
R^{A} represents —R^{A1} or —OR^{A1}
R^{A1} represents optionally substituted C₁₋₆ alkyl,
R^{A1} represents a hydrogen atom, optionally substituted C₁₋₆ alkyl, or C(O)—R^{b}, and
R^{B} represents optionally substituted C₁₋₆ alkyl (as used herein, sometimes referred to as “compound (1)” or the “compound represented by formula (1)”),
or a pharmaceutically acceptable salt thereof.
   (2) The compound according to (1) or a pharmaceutically acceptable salt thereof, wherein A¹ is methylene, and L¹ is optionally substituted methylene or optionally substituted ethylene.
   (3) The compound according to (1) or a pharmaceutically acceptable salt thereof, wherein A¹ is an oxygen atom, and L¹ is optionally substituted methylene or optionally substituted ethylene.
   (4) The compound according to (1) or a pharmaceutically acceptable salt thereof, wherein 1 is 1 or 2.
   (5) The compound according to (1) or a pharmaceutically acceptable salt thereof, wherein Z is —NR¹R².
   (6) The compound according to (1) or a pharmaceutically acceptable salt thereof, wherein Z is —OR³.
   (7) The compound according to (1) or a pharmaceutically acceptable salt thereof, wherein R¹ and R² each independently are a hydrogen atom or optionally substituted C₁₋₆ alkyl.
   (8) The compound according to (1) or a pharmaceutically acceptable salt thereof, wherein R¹ and R², together with the nitrogen atom to which they are attached, form an optionally substituted 4- to 7-membered saturated heterocycle.
   (9) The compound according to (1) or a pharmaceutically acceptable salt thereof, wherein R³ is a hydrogen atom or optionally substituted C₁₋₆ alkyl.
   (10) The compound according to (1) or a pharmaceutically acceptable salt thereof, wherein T is methyl optionally substituted with a halogen atom.
   (11) The compound according to any one of (1) to (10) or a pharmaceutically acceptable salt thereof, wherein the compound is selected from the group of the following compounds:
(3aR,4R,6aR)-1-(7,8-dihydrofuro[3,2-e][1,3]benzothiazol 2-yl)-2-oxooctahydrocyclcopenta[d]imidazol 4-yl rac-acetate (Example 6);
(3aR,4R,6aR)-3-(7,8-dihydrofuro[3,2-e][1,3]benzothiazol 2-yl)-2-oxooctahydrocyclopenta[d]imidazol 4-yl rac-acetate (Example 7);
rac-(3R,6R,6aR)-1-(7,8-dihydrofuro[3,2-e][1,3]benzothiazol 2-yl)-6-hydroxyhexahydrocyclopenta[d]imidazol 2 (1H)-one (Example 10);
rac-(3aR,4R,6aR)-1-(7,8-dihydrofuro[3,2-e][1,3]benzothiazol 2-yl)-4-hydroxyhexahydrocyclopenta[d]imidazol 2(1H)-one (Example 11);
rac-(3aR,4S,6aS)-1-(7,8-dihydrofuro[3,2-e][1,3]benzothiazol 2-yl)-4-(methylamino) hexahydrocyclopenta[d]imidazol 2 (1H)-one (Example 14);
rac-(3aR,4S,6aS)-1-(7,8-dihydrofuro 3,2-e][1,3]benzothiazol 2-yl)-4-(dimethylamino) hexahydrocyclopenta[d]imidazol 2 (1H)-one (Example 19);
rac-{[(3aR,4S,6aS)-1-(7,8-dihydrofuro[3,2-e][1,3]benzothiazol 2-yl)-2-oxooctahydrocyclopenta[d]imidazol 4-yl](methyl)amino}acetonitrile (Example 20);
rac-(3aR,6R,6aR)-1-(7,8-dihydrofuro[3,2-e][1,3]benzothiazol 2-yl)-6-(dimethylamino)hexahydrocyclopenta[d]imidazol 2(1H)-one (Example 21);
rac-{[(4R)-3-(7,8-dihydrofuro[3,2-e][1,3]benzothiazol 2-yl)-2-oxooctahydrocyclopenta[d]imidazol 4-yl](methyl)amino}acetonitrile (Example 22);
rac-{[(3aR,5R,6aS)-1-(7,8-dihydrofuro[3,2-e][1,3]benzothiazol 2-yl)-2-oxooctahydrocyclopenta[d]imidazol 5-yl](methyl)amino}acetonitrile (Example 30);
rac-{[(3aR,5R,6aS)-1-(7,8-dihydrofuro[3,2-e][1,3]benzothiazol 2-yl)-2-oxocetahydrocyclopenta[d]imidazol 5-yl]amino}acetonitrile (Example 31);
rac-(3aR,6R,6aR)-6-(3,3-difluoroazetidin-1-yl)-1-(7,8-dihyodrofuro [3,2-e][1,3]benzothiazol 2-yl) hexahydrocyclopenta[d]imidazol 2(1H)-one (Example 34);
(3aS,4R,6aR)-4-amino-1-(7,8-dihydrofuro[3,2-e][1,3]benzothiazol 2-yl)hexahydrocyclopenta[d]imidazol 2 (1H)-one (Example 37);
(3aS,4R,6aR)-4-(cyclopropylamino)-1-(7,8-dihydrofuro[3,2-e][1,3]benzothiazol-2-yl) hexahydrocyclopenta[d]imidazol 2(1H)-one (Example 40);
rac-(3aR,4S,6aS)-4-(3,3-difluoroazetidin-1-yl)-1-(7,8-dihydrofuro[3,2-e][1,3]benzothiazol-2-yl)hexahydrocyclopenta[d]imidazol-2 (1H)-one (Example 45);
rac-(3aR,6R,6aS)-6-(cyclopropylamino)-1-(7,8-dihydrofuro[3,2-e][1,3]benzothiazol-2-yl)hexahydrocyclopenta[d]imidazol-2 (1H)-one (Example 46);
rac-(3aR,6R,6aR)-1-(7,8-dihydrofuro[3,2-e][1,3]benzothiazol-2-yl)-6-(morpholin-4-yl)hexahydrocyclopenta[d]imidazol-2 (1)-one (Example 47);
rac-(3aR,6R,6aR)-6-[cyclopropyl(methyl)amino]-1-(7,8-dihydrofuro[3,2-e][1,3]benzothiazol-2-yl) hexahydrocyclopenta[d]imidazol-2 (1H)-one (Example 49);
(3aS,6S,6aS)-1-(7,8-dihydrofuro[3,2-e][1,3]benzothiazol-2-yl)-6-(dimethylamino) hexahydrocyclopenta[d]imidazol-2 (1H)-one (Example 50);
(3aR,4R,5S,6aR)-1-(7,8-dihydrofuro[3,2-e][1,3]benzothiazol-2-yl-4-(dimethylamino)-5-methylhexahydrocyclopenta[d]imidazol-2 (1H)-one (Example 57);
(3aR,4R,5S,6aR)-1-(7,8-dihydrofuro[3,2-e][1,3]-benzothiazol-2-yl)-4-(dimethylamino)-5-(fluoromethyl) hexahydrocyclopenta[d]imidazol-2(1H)-one (Example 58);
(3aR,4R,5S,6aR)-4-(dimethylamino)-1-(2H-[1,3]dioxolo[4,5-e][1,3]benzothiazol-7-yl)-5-methylhexahydrocyclopenta[d]imidazol-2(1H)-one (Example 59); and
(3aR,4R,5S,6aR)-4-(dimethylamino)-1-(2H-[1,3]dioxolo[4,5-e][1,3]benzothiazol-7-yl)-5-(fluoromethyl) hexahydrocyclopenta[d]imidazol-2 (1H)-one (Example 60).
   (12) A medicament comprising the compound according to any one of (1) to (11) or a pharmaceutically acceptable salt thereof as an active ingredient.
   (13) A pharmaceutical composition comprising the compound according to any one of (1) to (11) or a pharmaceutically acceptable salt thereof as an active ingredient.
   (14) A therapeutic agent and/or a prophylactic agent for a disease involving DYRK, comprising the compound according to any one of (1) to (11) or a pharmaceutically acceptable salt thereof as an active ingredient.
   (15) The therapeutic agent and/or the prophylactic agent according to (14), wherein the disease involving DYRK is frontotemporal dementia, progressive supranuclear palsy, corticobasal degeneration, Lewy body dementia, vascular dementia, traumatic brain injury, chronic traumatic encephalopathy, stroke, Alzheimer's disease, Parkinson's disease, Down's syndrome, or depression, and mental retardation, memory impairment, memory loss, learning disability, intellectual disability, cognitive dysfunction, mild cognitive impairment, or dementia symptom associated therewith, or brain tumor, pancreatic cancer, ovarian cancer, osteosarcoma, colorectal cancer, lung cancer, bone resorption disease, osteoporosis, sickle cell anemia, chronic renal disease, or bone resorption disease.
   (16) A method for treating and/or preventing a disease involving DYRK, comprising administration of a therapeutically effective amount of the compound according to any one of (1) to (11) or a pharmaceutically acceptable salt thereof to a patient in need of treatment.
   (17) Use of the compound according to any one of (1) to (11) or a pharmaceutically acceptable salt thereof, for producing a therapeutic agent and/or a prophylactic agent for a disease involving DYRK.
   (18) The compound according to any one of (1) to (11) or a pharmaceutically acceptable salt thereof, for use in treatment and/or prevention of a disease involving DYRK.
   (19) A medicament obtained by combining the medicament according to (12) and at least one or more agents selected from agents classified into an anticancer agent, an antipsychotic drug, an antidementia drug, an antiepileptic drug, an antidepressant drug, a gastrointestinal drug, a thyroid hormone drug, or an antithyroid drug.
   (20) The medicament according to (12), for treating frontotemporal dementia, progressive supranuclear palsy, corticobasal degeneration, Levy body dementia, vascular dementia, traumatic brain injury, chronic traumatic encephalopathy, stroke, Alzheimer's disease, Parkinson's disease, Down's syndrome, or depression, and complication, mental retardation, memory impairment, memory loss, learning disability, intellectual disability, cognitive dysfunction, mild cognitive impairment, or treating dementia symptom progression or preventing dementia onset associated therewith, or treating brain tumor, pancreatic cancer, ovarian cancer, osteosarcoma, colorectal cancer, lung cancer, bone resorption disease, osteoporosis, sickle cell anemia, chronic renal disease, or bone resorption disease, in combination with at least one or more agents selected from agents classified into an anticancer agent, an antipsychotic drug, an antidementia drug, an antiepileptic drug, an antidepressant drug, a gastrointestinal drug, a thyroid hormone drug, or an antithyroid drug.

### Effect of the Invention

The present inventors have carried out various studies in order to solve the above problems and as a result, have found that the amine derivative represented by the above formula (1) and a pharmaceutically acceptable salt thereof are an excellent group of drugs having an excellent DYRK inhibitory effect, and have completed the present invention. The compound provided by the present invention is useful as a pharmaceutical (pharmaceutical composition) for prevention or treatment of a disease known to be associated with a DYRK1A-mediated abnormal cellular response, such as a psychiatric or neurologic disease such as Alzheimer's disease, Parkinson's disease, Down's syndrome, or depression, and mental retardation, memory impairment, memory loss, learning disability, intellectual disability, cognitive dysfunction, mild cognitive impairment, or a therapeutic drug for dementia symptom progression or a prophylactic drug for dementia onset associated therewith, or further a tumor such as brain tumor. The compound provided by the present invention is, as an inhibitor of DYRK1B, useful as a pharmaceutical (pharmaceutical composition) for prevention or treatment of a tumor such as pancreatic cancer, ovarian cancer, osteosarcoma, colorectal cancer, or lung cancer. Further, the compound provided by the present invention is useful as a pharmaceutical (pharmaceutical composition) for prevention or treatment of bone resorption disease and osteoporosis because DYRK2 controls p53 in response to DNA damage to induce apoptosis. In addition, the compound provided by the present invention is, as an inhibitor of DYRK3, useful as a pharmaceutical (pharmaceutical composition) for prevention or treatment of sickle cell anemia, chronic renal disease, bone resorption disease, and osteoporosis. In addition, the compound provided by the present invention is, as a compound that inhibits DYRK, useful as a reagent for pathological imaging related to the above diseases or a reagent for a basic experiment or for research.

### BEST MODE TO CARRY OUT THE INVENTION

The terms used herein will be described below.

“DYRK” stands for Dual-specificity tYrosine-phosphorylation Regulated protein Kinase, and means one or two or more of the DYRK family (DYRK1A, DYRK1B, DYRK2, DYRK3, and DYRK4).

Examples of a “halogen atom” include a fluorine atom, a chlorine atom, a bromine atom, or an iodine atom. The halogen atom is preferably a fluorine atom.

“C₁₋₆ alkyl” means a linear or branched saturated hydrocarbon group having 1 to 6 carbon atoms, and “C₆ alkyl” means a linear or branched saturated hydrocarbon group having 6 carbon atoms. The same also applies to other numbers. The C₁₋₆ alkyl is preferably “C₁₋₄ alkyl” and more preferably “C₁₋₃ alkyl.” Specific examples of the “C₁₋₃ alkyl” include methyl, ethyl, propyl, and 1-methylethyl. Specific examples of the “C₁₋₄ alkyl” include butyl, 1,1-dimethylethyl, 1-methylpropyl, and 2-methylpropyl, in addition to those given as specific examples of the “C₁₋₃ alkyl.” Specific examples of the “C₁₋₆ alkyl” include pentyl, 1,1-dimethylpropyl, 1,2-dimethylpropyl, 1-methylbutyl, 2-methylbutyl, 4-methylpentyl, 3-methylpentyl, 2-methylpentyl, 1-methylpentyl, and hexyl, in addition to those given as specific examples of the “C₁₋₄ alkyl.”

“C₁₋₆ alkoxy” means “C₁₋₆ alkyloxy,” and the “C₁₋₆ alkyl” moiety is defined as the “C₁₋₆ alkyl.” The “C₁₋₆ alkoxy” is preferably “C₁₋₄ alkoxy” and more preferably “C₁₋₃ alkoxy.” Specific examples of the “C₁₋₃ alkoxy” include methoxy, ethoxy, propoxy, and 1-methylethoxy. Specific examples of the “C₁₋₄ alkoxy” include butoxy, 1,1-dimethylethoxy, 1-methtylpropoxy, and 2-methylpropoxy, in addition to those given as specific examples of the “C₁₋₃ alkoxy.” Specific examples of the “C₁₋₆ alkoxyl” include pentyloxy, 1,1-dimethylpropoxy, 1,2-dimethylpropoxy, 1-methylbutoxy, 2-methylbutoxy, 4-methylpentyloxy, 3-methylpentyloxy, 2-methylpentyloxy, 1-methylpentyloxy, and hexyloxy, in addition to those given as specific examples of the “C₁₋₄ alkoxy.”

“C₃₋₁₀ cycloalkyl” means a cyclic saturated hydrocarbon group having 3 to 0 carbon atoms, and also includes one having partially an unsaturated bond and one having no a crosslinked structure. The “C₃₋₁₀ cycloalkyl” is preferably “C₃₋₇ cycloalkyl.” Specific examples of the “C₃₋₇ cycloalkyl” include cyclopropyl, cyclobutyl, cyclopentyl, cyclohexyl, and cycloheptyl. Specific examples of the “C₃₋₁₀ cycloalkyl” include cyclooctyl, cyclononyl, cyclodecyl, and adamantyl, in addition to those given as specific examples of the “C₃₋₇ cycloalkyl.”

A “4- to 7-membered saturated heterocycle” means a monocyclic or bicyclic saturated heterocycle containing one or more heteroatoms selected from the group of a nitrogen atom, an oxygen atom, and a sulfur atom, and also includes one having partially an unsaturated bond and one having a crosslinked structure. The “4- to 7-membered saturated heterocycle” is preferably a “4- to 6-membered saturated heterocycle,” more preferably a “5- or 6-membered saturated heterocycle.” Specific examples of the “4- to 7-membered saturated heterocycle” include an azetidine ring, a pyrrolidine ring, a piperidine ring, an azepane ring, a morpholine ring, a piperazine ring, an azabicycloheptane ring, an oxetane ring, a thietane ring, a tetrahydrofuran ring, a tetrahydrothiophene ring, a tetrahydropyran ring, a thiomorpholine ring, and a 1,4-dioxane ring.

In the structural formula represented by formula (1), the tricyclic heterocycle formed by including A¹ and L¹ represents a chemically stable heterocycle. The tricyclic heterocycle preferably has the structure shown below.

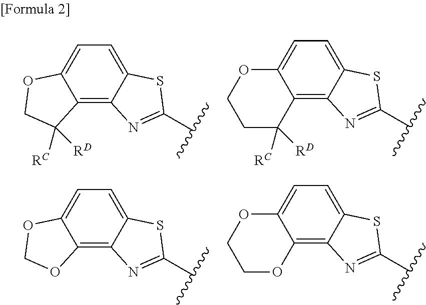

In the compound of the present invention represented by formula (1), the definitions and preferred ranges of A¹, L¹, R¹, R², R³, R^{A}, R^{B}, R^{A1}, l, T, and Z are as follows, and the technical scope of the present invention is not limited to the scope of compounds listed below.

A¹ is optionally substituted methylene or an oxygen atom.

L¹ is optionally substituted methylene or optionally substituted ethylene, and preferably methylene or ethylene.

R¹ and R² are each independently a hydrogen atom, optionally substituted C₁₋₆ alkyl, or C(O)—R^{A}, and preferably a hydrogen atom or optionally substituted C₁₋₆ alkyl. Alternatively, R¹ and R², together with the nitrogen atom to which R¹ and R² are attached, may form an optionally substituted 4- to 7-membered saturated heterocycle, and preferably form a 4- or 6-membered saturated heterocycle.

R^{A} is —R^{A1} or —OR^{A1}, and R^{A1} is optionally substituted C₁₋₆ alkyl, and preferably trifluoromethyl or tert-butyl.

R¹ is a hydrogen atom, optionally substituted C₁₋₆ alkyl, or C(O)—R³ and preferably a hydrogen atom or optionally substituted C₁₋₆ alkyl.

R³ is optionally substituted C₁₋₆ alkyl, preferably methyl.

l is 1, 2, or 3 and preferably 1 or 2.

Z is —NR¹R² or —OR³, and preferably —NR¹R².

T is a hydrogen atom or optionally substituted C₁₋₆ alkyl, preferably a hydrogen atom or C₁₋₆ alkyl optionally substituted with a halogen atom, more preferably a hydrogen atom or methyl optionally substituted with a halogen atom, and further preferably a hydrogen atom, methyl, or monofluoromethyl.

Substitution with Z and T can occur on any identical or different carbon atoms present on a 5- to 7-membered carbocycle of the compound represented by formula (1), other than the bonding position between the carbocycle and a ring to which the carbocycle is fused.

As used herein, the substituent when the “optionally substituted C₁₋₆ alkyl” is substituted is one or more substituents selected from the group consisting of a halogen atom, hydroxy, and optionally substituted C₃₋₁₀; cycloalkyl and optionally substituted C₁₋₆ alkoxy, and substitution with such a substituent occurs at any substitutable position. The number of the substituents is preferably 1 to 5, and more preferably 1 to 3. When substitution with two or more substituents occurs, these substituents may be the same or different.

As used herein, the substituent when the “optionally substituted C₃₋₁₀ cycloalkyl” is substituted is one or more substituents selected from the group consisting of a halogen atom, hydroxy, C₁₋₆ alkyl, C₁₋₆ alkoxy, and a C₃₋₁₀ cycloalkyl group, and substitution with such a substituent occurs at any substitutable position. The number of the substituents is preferably 1 to 5, and more preferably 1 to 3. When substitution with two or more substituents occurs, these substituents may be the same or different.

As used herein, the substituent when the “optionally substituted C₁₋₆ alkoxy” is substituted is one or more substituents selected from the group consisting of a halogen atom, hydroxy, C₁₋₆ alkyl, C₁₋₆ alkoxy, and a C₃₋₈ cycloalkyl group, and substitution with such a substituent occurs at any substitutable position. The number of the substituents is preferably 1 to 5, and more preferably 1 to 3. When substitution with two or more substituents occurs, these substituents may be the same or different.

As used herein, the substituent when the “optionally substituted methylene” is substituted is one or more substituents selected from C₁₋₆ alkyl, and substitution with such a substituent occurs at any substitutable position. The number of the substituents is preferably 1 to 4. When substitution with two or more substituents occurs, these substituents may be the same or different, and two substituents on the same carbon atom, together with the carbon atom to which they are attached, may form a spiro ring consisting of a 4- to 8-membered saturated heterocycle or a 3- to 8-membered saturated carbocycle.

As used herein, the substituent when the “optionally substituted ethylene” is substituted is one or more substituents selected from the group consisting of C₁₋₆ alkyl and an oxo group, and substitution with such a substituent occurs at any substitutable position. The number of the substituents is preferably 1 to 4. When substitution with two or more substituents occurs, these substituents may be the same or different, and two substituents on the same carbon atom, together with the carbon atom to which they are attached, may form a spiro ring consisting of a 4- to 8-membered saturated heterocycle or a 3- to 8-membered saturated carbocycle.

As used herein, the substituent that the “optionally substituted 4- to 7-membered saturated heterocycle” optionally has is one or more substituents selected from the group consisting of a halogen atom, hydroxy, C₁₋₆ alkyl, C₁₋₆ alkoxy, and C₃₋₈ cycloalkyl, and substitution with such a substituent occurs at any substitutable position. The number of the substituents is preferably 1 to 5, and more preferably 1 to 3. When substitution with two or more substituents occurs, these substituents may be the same or different, and two substituents on the same carbon atom on the ring, together with the carbon atom to which they are attached, may form a spiro ring consisting of a 4- to 8-membered saturated heterocycle or a 3- to 8-membered saturated carbocycle, or two substituents on different carbon atoms on the ring may combine to form a crosslink.

Among the compounds of the present invention represented by formula (1), examples of a preferred compound include the following compounds or pharmaceutically acceptable salts thereof.

A compound wherein A¹ and L¹ are methylene, and R¹, R², and R³ are a hydrogen atom or optionally substituted C₁₋₄ alkyl.

A compound wherein A¹ and L¹ are methylene, and R¹ and R² are a hydrogen atom or optionally substituted C₁₋₆ alkyl.

A compound wherein A¹ and L¹ are methylene, and R³ is a hydrogen atom or optionally substituted C₁₋₆ alkyl.

A compound wherein A¹ and L¹ are methylene, and R¹ and R², together with the nitrogen atom to which they are attached, form an optionally substituted 4- to 7-membered saturated heterocycle.

A compound wherein A¹ and L¹ are each ethylene, and R¹, R², and R³ are each a hydrogen atom or optionally substituted C₁₋₆ alkyl.

A compound wherein A¹ and L¹ are each ethylene, and R¹ and R² are each a hydrogen atom or optionally substituted C¹⁻⁶ alkyl.

A compound wherein A¹ and L¹ are each ethylene, and R³ is a hydrogen atom or optionally substituted C¹⁻⁶ alkyl.

A compound wherein A¹ and L¹ are each ethylene, and R¹ and R², together with the nitrogen atom to which they are attached, form an optionally substituted 4- to 7-membered saturated heterocycle.

A compound wherein A¹ is an oxygen atom, L¹ is methylene or ethylene, and R¹, R², and R³ are each a hydrogen atom or optionally substituted C¹⁻⁶ alkyl.

A compound wherein A¹ is an oxygen atom, L¹ is methylene or ethylene, and R¹ and R² are each a hydrogen atom or optionally substituted C₁₋₆ alkyl.

A compound wherein A¹ is an oxygen atom, L¹ is methylene or ethylene, and R³ is a hydrogen atom or optionally substituted C₁₋₆ alkyl.

A compound wherein A¹ is an oxygen atom, L¹ is methylene or ethylene, and R¹ and R², together with the nitrogen atom to which they are attached, form an optionally substituted 4- to 7-membered saturated heterocycle.

A compound wherein A¹ is an oxygen atom, L¹ is methylene, and R¹, R², and R³ are each a hydrogen atom or optionally substituted C₁₋₆ alkyl.

A compound wherein A¹ is an oxygen atom, L¹ is methylene, and R¹ and R² are each a hydrogen atom or optionally substituted C₁₋₆ alkyl.

A compound wherein A¹ is an oxygen atom, L¹ is methylene, and R³ is a hydrogen atom or optionally substituted C₁₋₆ alkyl.

A compound wherein A¹ is an oxygen atom, L¹ is methylene, and R¹ and R², together with the nitrogen atom to which they are attached, form an optionally substituted 4- to 7-membered saturated heterocycle.

Hereinafter, the method for producing the compound represented by formula (1) in the present invention will be described with reference to examples, but the present invention is not limited thereto.

The compound of the present invention is synthesized by a production method shown below and a method combining a known compound and a known synthesis method.

Each of the compounds in a reaction scheme also includes a salt thereof, and examples of the salt include the same as a salt of compound (1). These reactions are merely examples, and the compound of the present invention can also be appropriately produced by other methods based on the knowledge of a person who is familiar with organic synthesis.

In each of the production methods described below, even if the use of a protective group is not specifically specified, when a functional group that requires protection is present, the target product may be obtained by, if necessary, protecting the functional group and deprotecting the same after completion of the reaction or after carrying out a series of reactions.

As the protective group, a usual protective group disclosed in, for example, a reference (T. W. Greene and P. G. M. Wuts, “Protective Groups in Organic Synthesis,” 3rd Ed., John Wiley and Sons, inc., New York (1999)) can be used, and more specifically, examples of a protective group for an amino group include an alkyloxycarbonyl such as tert-butoxycarbonyl, benzyloxycarbonyl, or trimethylsilylethyloxycarbonyl, dimethylformamide, trifluoroacetyl, p-toluenesulforyl, o-nitrobenzenesulfonyl, benzyl, and tetrahydropyranyl, examples of a protective group for a hydroxy group include a trialkylsilyl, acetyl, benzyl, tetrahydropyranyl, methoxymethyl, and a dialkyl acetal, examples of a protective group for an aldehyde group include a dialkyl acetal, and a cyclic alkyl acetal, and examples of a protective group for a carboxyl group include a tert-butyl ester, an ortho ester, and an acid amide.

The introduction and elimination of a protective group can be carried out by a method commonly used in organic synthetic chemistry (for example, a method described in T. W. Greene and P. G. M. Wuts, “Protective Groups in Organic Synthesis,” 3rd Ed., John Wiley and Sons, inc., New York (1999)) or a method similar thereto.

### Production Method 1

The compound represented by formula (1-5) is produced, for example, by the method shown below.

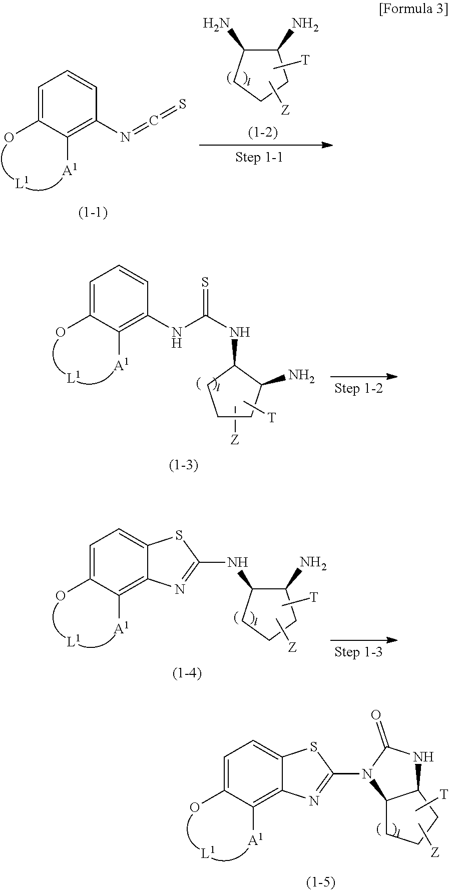

wherein A¹, L¹, T, Z, and 1 are defined as described in item 1 above.

### Step 1-1: Production Step of Compound (1-3)

Compound (1-3) can be produced by reacting compound (1-1) with compound (1-2) by a method similar to a known synthesis method (for example, Chemical & Pharmaceutical Bulletin, 1406, (2007), or Advanced Synthesis & Catalysis, 1643, (2005)). As compound (1-1), a compound produced by a known synthesis method (for example, Bioorganic & Medicinal Chemistry Letters, 28, (2007), or J. Org. Chem. 2613, (1986)) or a synthesis method similar thereto can be used. As compound (1-2), a commercially available product or a compound produced by a known synthesis method (for example, WO2014144737 or US20050020645) or a synthesis method similar thereto or by production method 3 or production method 4 can be used.

### Step 1-2: Production Step of Compound (1-4)

Compound (1-4) is produced by cyclizing compound (1-3) by a method similar to a known synthesis method (for example, Journal of Organic Chemistry, 8693, (2003) or WO2013043001).

### Step 1-3: Production Step of Compound (1-5)

Compound (1-5) is produced by removing a protective group, if necessary, and then cyclizing compound (1-4) by a method similar to a known synthesis method (for example, Organic Letters, 5136, (2015), or Bioorganic & Medicinal Chemistry, 822, (2008)).

### Production Method 2

The compound represented by formula (2-5) is produced, for example, by the method shown below.

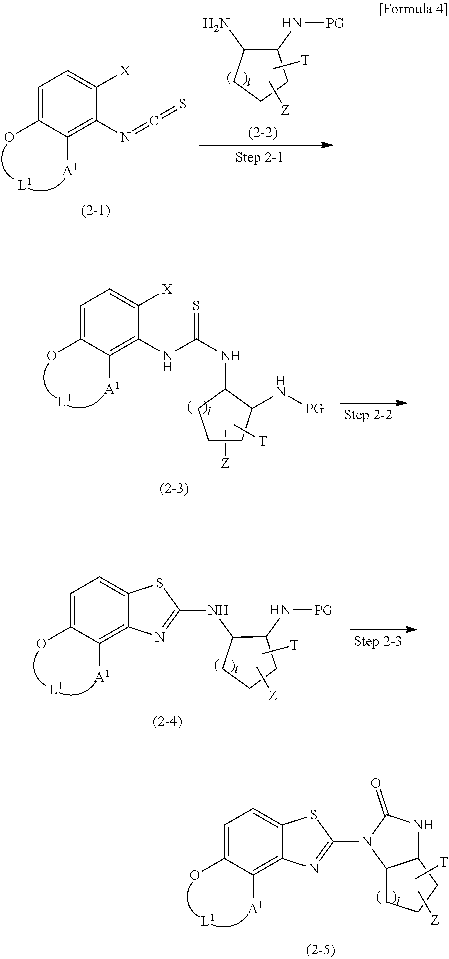

wherein A¹, L¹, T, Z, and 1 are defined as described in item 1 above; X represents a halogen atom (for example, an iodine atom, a bromine atom, or a chlorine atom); PG represents a protective group (for example, an alkyloxycarbonyl group such as a tert-butoxycarbonyl group, a benzyloxycarbonyl group, a trifluoromethylcarbonyl group, or a trimethylsilylethylcarbonyl group.

### Step 2-1: Production Step of Compound (2-3)

Compound (2-3) is produced according to the method described in step 1-1 by using compound (2-1) and compound (2-2). As compound (2-1), a compound produced by a known synthesis method (for example, Bioorganic & Medicinal Chemistry Letters, 28, (2007) or Journal of Organic Chemistry 2613, (1986)) or a synthesis method similar thereto can be used. As compound (2-2), a commercially available product or a compound produced by a known synthesis method (for example, Bioorganic & Medicinal Chemistry Letters 597, (2009), or WO2007003596) or a synthesis method similar thereto or by production method 3 or production method 4 can be used.

### Step 2-2: Production Step of Compound (2-4)

Compound (2-4) is produced by using compound (2-3) and cyclizing the same by a method similar to a known synthesis method (for example, Chemical Communications 446, (2004) or Journal of Organic Chemistry 8719, (2009)). Step 2-1 and step 2-2 can also be carried out as one step at a time.

Step 2-3: Production step of compound (2-5) Compound (2-5) is produced by using compound (2-4) and according to the method described in step 1-3 after deprotection of the protective group.

### Production Method 3

The compounds represented by formulas (3-6) and (3-7) are produced, for example, by the method shown below.

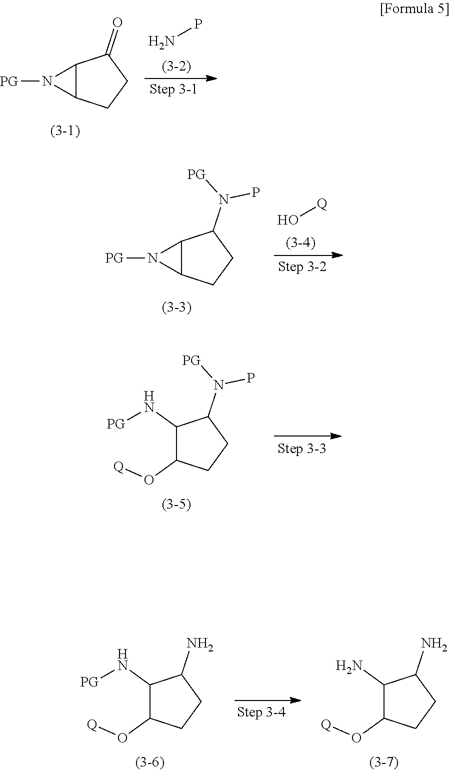

wherein P represents a protective group (for example, a benzyl group or an optionally substituted benzyl group such as a p-methoxybenzyl group); Q represents optionally substituted C₁₋₆ alkylcarbonyl; and PG represents a protective group (for example, an alkyloxycarbonyl group such as a tert-butoxycarbonyl group, a benzyloxycarbonyl group, a trifluoromethylcarbonyl group, or a trimethylsilylethylcarbonyl group.

### Step 3-1: Production Step of Compound (3-3)

Compound (3-3) is produced by reacting compound (3-1) with compound (3-2) in an inert solvent in the presence of a borohydride compound and, if necessary, an acid, and then protecting the resulting amino group. As compound (3-1), a compound produced by a known synthesis method (for example, Tetrahedron, 1991 (2016), or Organic Letters 2347 (2016)) or a synthesis method similar thereto can be used. As compound (3-2), a commercially available product or a compound produced by a known synthesis method (for example, Bulletin of the Chemical Society of Japan 2797 (1971), or Organic & Biomolecular Chemistry 6600 (2018)) or a synthesis method similar thereto can be used.

Specific examples of the inert solvent include an ether-based solvent such as tetrahydrofuran, tetrahydropyran, 1,4-dioxane, or 1,2-dimethoxyethane; a halogenated hydrocarbon such as chloroform, dichloromethane, or 1,2-dichloroethane; a protic polar solvent such as methanol, ethanol, 1-propanol, 2-propanol, or water; and mixed solvents thereof. The inert solvent is preferably tetrahydrofuran, dichloromethane, chloroform, or methanol.

Specific examples of the acid include a carboxylic acid such as formic acid, propionic acid, acetic acid, or trifluoroacetic acid; and a mineral acid such as hydrochloric acid.

Specific examples of the borohydride compound include sodium triacetoxyborohydride, sodium cyanoborohydride, and sodium borohydride. The borohydride compound is preferably sodium triacetoxyborohydride or sodium cyanoborohydride.

The reaction temperature is not particularly limited, and is usually selected from the range from 0° C. to the boiling point of the solvent used. The reaction temperature is preferably 0° C. to 20° C. The reaction time is usually 30 minutes to 72 hours.

Compound (3-3) can also be produced by reacting compound (3-1) with compound (3-2) in an inert solvent in the presence of, if necessary, an acid, under a catalytic hydrogen reduction condition using a metal catalyst, and protecting the resulting amino group.

Specific examples of the inert solvent include an ether-based solvent such as tetrahydrofuran, tetrahydropyran, 1,4-dioxane, or 1,2-dimethoxyethane; an ester-based solvent such as ethyl acetate or isopropyl acetate; a protic polar solvent such as water, methanol, ethanol, or isopropanol; and mixed solvents thereof.

Specific examples of the acid include a carboxylic acid such as formic acid, propionic acid, acetic acid, or trifluoroacetic acid; and a mineral acid such as hydrochloric acid.

Specific examples of the metal catalyst include palladium/carbon, palladium hydroxide/carbon, Raney nickel/carbon, platinum oxide/carbon, and rhodium/carbon. The amount of the metal catalyst used is usually 0.1 to 1000% by weight, and preferably 1 to 100% by weight, based on the amount of compound (3-1).

The hydrogen pressure is not particularly limited, and is usually about 1 to about 100 atmospheres, and preferably about 1 to about 5 atmospheres. The reaction temperature is not particularly limited, and is usually 0° C. to 120° C., and preferably 20° C. to 80° C. The reaction time is usually 30 minutes to 72 hours, and preferably 1 hour to 24 hours.

### Step 3-2: Production Step of Compound (3-5)

Compound (3-5) is produced by reacting compound (3-3) with compound (3-4) in, if necessary, an inert solvent by a method similar to a known synthesis method (for example, Organic Letters 2347 (2016), or Tetrahedron 5849 (2014)). As compound (3-4), a commercially available product or a compound produced by a known synthesis method (for example, RSC Advances 6606 (2013), or Angewandte Chemie, International Edition 5772 (2008)) or a synthesis method similar thereto can be used.

Specific examples of the inert solvent include an ether-based solvent such as tetrahydrofuran, tetrahydropyran, 1,4-dioxane, or 1,2-dimethoxyethane; an aprotic polar solvent such as acetonitrile, N,N-dimethylformamide, N-methyl-2-pyrrolidinone, or dimethyl sulfoxide; a protic polar solvent such as water, methanol, ethanol, or isopropanol; and mixed solvents thereof. No solvent, methanol, and ethanol are preferable.

### Step 3-3: Production Step of Compound (3-6)

Compound (3-6) is produced by using compound (3-5), using a method similar to a known synthesis method (for example, Journal of Medicinal Chemistry 6916 (2012), or Journal of the American Chemical Society 4649 (1987)), and further, if necessary, deprotecting the protecting group.

### Step 3-4: Production Step of Compound (3-7)

Compound (3-7) is produced by deprotecting the protective group of compound (3-6). Step 3-3 and step 3-4 can also be carried out as one step at a time.

### Production Method 4

The compound represented by formula (4-3) is produced, for example, by the method shown below.

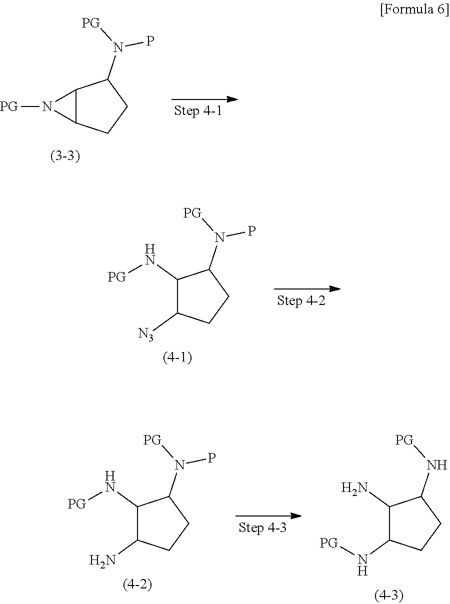

wherein P represents optionally substituted C₁₋₆ alkyl; and PG represents a protective group (for example, an alkyloxycarbonyl group such as tert-butoxycarbonyl group, a benzyloxycarbonyl group, a trifluoromethylcarbonyl group, or a trimethylsilylethylcarbonyl group.

### Step 4-1: Production Step of Compound (4-1)

Compound (4-1) is produced by reacting compound (3-3) with an azide compound in an inert solvent, in the presence of, if necessary, an acid.

Specific examples of the inert solvent include an ether-based solvent such as tetrahydrofuran, tetrahydropyran, 1,4-dioxane, or 1,2-dimethoxyethane; a halogenated hydrocarbon such as chloroform, dichloromethane, or 1,2-dichloroethane; an aprotic polar solvent such as acetonitrile, N,N-dimethylformamide, N-methyl-2-pyrrolidinone, or dimethyl sulfoxide; a protic polar solvent such as methanol, ethanol, 1-propanol, 2-propanol, or water; and mixed solvents thereof. The inert solvent is preferably dichloromethane, chloroform, methanol, or 2-propanol.

Specific examples of the acid include a carboxylic acid such as formic acid, propionic acid, acetic acid, or trifluoroacetic acid; a mineral acid such as hydrochloric acid; and a Lewis acid such as a boron trifluoride diethyl ether complex. The acid is preferably acetic acid or a boron trifluoride diethyl ether complex.

Specific examples of the azide compound include sodium azide, tetrabutylammonium azide, and trimethylsilyl azide. The azide compound is preferably sodium azide or trimethylsilyl azide. A combination of sodium azide and acetic acid or trimethylsilyl azide and a boron trifluoride diethyl ether complex is preferably used.

The reaction temperature is not particularly limited, and is usually selected from the range from 0° C. to the boiling point of the solvent used. The reaction temperature is preferably 0° C. to 80° C. The reaction time is usually 30 minutes to 72 hours.

### Step 4-2: Production Step of Compound (4-2)

Compound (4-2) is produced by using compound (4-1) and using a method similar to a known synthesis method (for example, WO2012173689, or Journal of the American Chemical Society 4281 (2004)).

### Step 4-3: Production Step of Compound (4-3)

Compound (4-3) is produced by using compound (4-2), using the method described in step 3-3, and detaching the protective group.

### Production Method 5

The compound represented by formula (5-4) is produced, for example, by the method shown below.

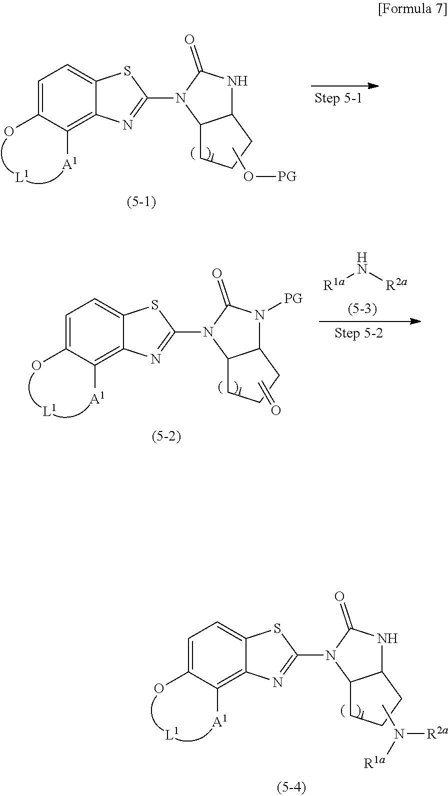

wherein A¹, L¹, and 1 are defined as described in item 1 above; R^{1a} and R^{2a} each independently represent a hydrogen atom or optionally substituted C₁₋₆ alkyl, or R^{1a} and R^{2a}, together with the nitrogen atom to which they are attached optionally may form an optionally substituted 4- to 7-membered saturated heterocycle; and PG represents a protective group (for example, an alkyloxycarbonyl such as a tert-butoxycarbonyl group, a benzyloxycarbonyl group, a triflucromethylcarbonyl group, or a trimethylsilylethylcarbonyl group, or an acetyl group.)

### Step 5-1: Production Step of Compound (5-2)

Compound (5-2) is produced by using a method similar to a known synthesis method (for example, WO2016096686, or Bioorganic & Medicinal Chemistry Letters 1917 (2000)) after detaching the protective group of Compound (5-1).

### Step 5-2: Production Step of Compound (5-4)

Compound (5-4) is produced by reacting compound (5-2) with compound (5-3) in an inert solvent in the presence of a borohydride compound and, if necessary, an acid. As compound (5-3), a commercially available product or a compound produced by a known synthesis method (for example, Tetrahedron Letters 3483 (1992), or Journal of Medicinal Chemistry 2213 (2014)) or a synthesis method similar thereto can be used.

Specific examples of the inert solvent include an ether-based solvent such as tetrahydrofuran, tetrahydropyran, 1,4-dioxane, or 1,2-dimethoxyethane; a halogenated hydrocarbon such as chloroform, dichloromethane, or 1,2-dichloroethane; a protic polar solvent such as methanol, ethanol, 1-propanol, 2-propanol, or water; and mixed solvents thereof. The inert solvent is preferably tetrahydrofuran, dichloromethane, chloroform, or methanol.

Specific examples of the acid include a carboxylic acid such as formic acid, propionic acid, acetic acid, or trifluoroacetic acid; and a mineral acid such as hydrochloric acid. The acid is preferably acetic acid.

Specific examples of the borohydride compound include sodium triacetoxyborohydride, sodium cyanoborohydride, and sodium borohydride. The borohydride compound is preferably sodium triacetoxyborohydride or sodium cyanoborohydride.

The reaction temperature is not particularly limited, and is usually selected from the range from 0° C. to the boiling point of the solvent used. The reaction temperature is preferably 0° C. to 20° C., or 50° C. to 70° C. The reaction time is usually 30 minutes to 72 hours.

In the present reaction, the stereoselectivity of compound (5-4) obtained can be changed depending on the combination of the inert solvent used and the reaction temperature.

Compound (5-4) can also be produced by reacting compound (5-2) with compound (5-3) in an inert solvent in the presence of, if necessary, an acid, under a catalytic hydrogen reduction condition using a metal catalyst.

Specific examples of the inert solvent include an ether-based solvent such as tetrahydrofuran, tetrahydropyran, 1,4-dioxane, or 1,2-dimethoxyethane; an ester-based solvent such as ethyl acetate or isopropyl acetate; a protic polar solvent such as water, methanol, ethanol, or isopropanol; and mixed solvents thereof.

Specific examples of the acid include a carboxylic acid such as formic acid, propionic acid, acetic acid, or trifluoroacetic acid; and a mineral acid such as hydrochloric acid.

Specific examples of the metal catalyst include palladium/carbon, palladium hydroxide/carbon, Raney nickel/carbon, platinum oxide/carbon, and rhodium/carbon. The amount of the metal catalyst used is usually 0.1 to 1000% by weight, and preferably 1 to 100% by weight, based on that of compound (3-1).

The hydrogen pressure is not particularly limited, and is usually about 1 to about 100 atmospheres, and preferably about 1 to about 5 atmospheres. The reaction temperature is not particularly limited, and is usually 0° C. to 120° C., and preferably 20° C. to 80° C. The reaction time is usually 30 minutes to 72 hours, and preferably 1 hour to 24 hours.

### Production Method 6

The compound represented by formula (6-3) is produced, for example, by the method shown below.

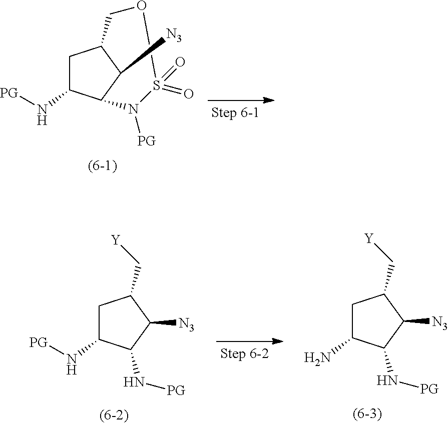

wherein Y represents a hydrogen atom or a fluorine atom; and PG represents a protective group (for example, an alkyloxycarbonyl group such as a tert-butoxycarbonyl group, a methoxycarbonyl group, an ethoxycarbonyl group, a benzyloxycarbonyl group, or a trimethylsilylethyloxycarbonyl group.

### Step 6-1: Production Step of Compound (6-2)

Compound (6-2) is produced by reacting compound (6-1) with a hydride reducing agent or a fluorinating agent in an inert solvent, in the presence of, if necessary, an additive. Compound (6-1) is produced by using a method similar to a known synthesis method (for example, Angewandte Chemie International Edition 3802 (2009)).

Specific examples of the inert solvent include an ether-based solvent such as tetrahydrofuran, tetrahydropyran, 1,4-dioxane, or 1,2-dimethoxyethane; a halogenated hydrocarbon such as chloroform, dichloromethane, or 1,2-dichloroethane; an aprotic polar solvent such as acetonitrile, N,N-dimethylformamide, N-methyl-2-pyrrolidinone, or dimethyl sulfoxide; a protic polar solvent such as methanol, ethanol, 1-propanol, 2-propanol, or water; and mixed solvents thereof. The inert solvent is preferably dimethyl sulfoxide, tetrahydrofuran, or acetonitrile.

Specific examples of the additive include a crown ether such as 12-crown-4, 15-crown-5, 18-crown-6, dibenzo-18-crown-6, or diaza-18-crown-6. The additive is preferably 18-crown-6.

Specific examples of the hydride reducing agent include sodium borohydride, lithium borohydride, lithium triethylborohydride, diisobutylaluminum hydride, and lithium aluminum hydride. The hydride reducing agent is preferably sodium borohydride.

Specific examples of the fluorinating agent include tetra-n-butylammonium fluoride, potassium fluoride, and cesium fluoride. The fluorinating agent is preferably tetra-n-butylammonium fluoride or potassium fluoride. A combination of tetra-n-butylammonium fluoride or potassium fluoride and 18-crown-6 is preferably used.

The reaction temperature is not particularly limited, and is usually selected from the range from 0° C. to the boiling point of the solvent used. The reaction temperature is preferably 0° C. to 80° C. The reaction time is usually 30 minutes to 72 hours.

### Step 6-2: Production Step of Compound (6-3)

Compound (6-3) is produced by using compound (6-2) and using a method similar to a known synthesis method (for example, Angewandte Chemie International Edition 3802 (2009), or Journal of Organic Chemistry 5137 (2011)).

By carrying out the above production methods in an appropriate combination, the compound of the present invention having a desired functional group at a desired position can be obtained. Isolation and purification of intermediates and products in the above production methods can be carried out by appropriately combining methods used in ordinary organic synthesis, such as filtration, extraction, washing, drying, concentration, crystallization, and various chromatography. In addition, such an intermediate can also be subjected to the next reaction without any particular purification.

Examples of the “pharmaceutically acceptable salt” include an acid addition salt and a base addition salt. Examples of the acid addition salt include an inorganic acid salt such as a hydrochloride, a hydrobromide, a sulfate, a hydroiodide, a nitrate, or a phosphate, or an organic acid salt such as a citrate, an oxalate, a phthalate, a fumarate, a maleate, a succinate, a malate, an acetate, a formate, a propionate, a benzoate, a trifluoroacetate, a methanesulfonate, a benzenesulfonate, a para-toluenesulfonate, or a camphorsulfonate. In addition, examples of the base addition salt include an inorganic base salt such as a sodium salt, a potassium salt, a calcium salt, a magnesium salt, a barium salt, or an aluminum salt, or an organic base salt such as trimethylamine, triethylamine, pyridine, picoline, 2,6-lutidine, ethanolamine, diethanolamine, triethanolamine, tromethamine [tris(hydroxymethyl)methylamine], tert-butylamine, cyclohexylamine, dicyclohexylamine, or N,N-dibenzylethylamine. Further, examples of the “pharmaceutically acceptable salt” also include an amino acid salt with a basic amino acid or an acidic amino acid, such as arginine, lysine, ornithine, aspartic acid, or glutamic acid.

Suitable salts of a raw material compound and an intermediate and a salt acceptable as a raw material for a pharmaceutical are conventional nontoxic salts, and examples thereof include an acid addition salt such as an organic acid salt (for example, an acetate, a trifluoroacetate, a maleate, a fumarate, a citrate, a tartrate, a methanesulfonate, a benzenesulfonate, a formate, or a p-toluenesulfonate) and an inorganic acid salt (for example, a hydrochloride, a hydrobromide, a hydroiodide, a sulfate, a nitrate, or a phosphate), a salt with an amino acid (for example, arginine, aspartic acid, or glutamic acid), a metal salt such as an alkali metal salt (for example, a sodium salt or a potassium salt) and an alkaline earth metal salt (for example, a calcium salt or a magnesium salt), an ammonium salt, or an organic base salt (for example, a trimethylamine salt, a triethylamine salt, a pyridine salt, a picoline salt, a dicyclohexylamine salt, or an N,N′-dibenzylethylenediamine salt), and such a nontoxic salt can be appropriately selected by those skilled in the art.

Some of the raw material compounds or intermediates in the above production methods can exist in the form of a salt such as a hydrochloride depending on the reaction conditions and the like, and can be used as they are or in free form. When a raw material compound or an intermediate is obtained in the form of a salt and the raw material compound or intermediate is to be used or obtained in free form, this salt can be converted to a free form by dissolving or suspending the salt in a suitable solvent and neutralizing the same with, for example, a base such as a sodium hydrogen carbonate aqueous solution.

For some compounds (1) or pharmaceutically acceptable salts thereof, an isomer such as a tautomer such as a keto-enol form, a regioisomer, a geometric isomer, or an optical isomer can exist, and all possible isomers, including these, and mixtures of the isomers at any ratio are also encompassed by the present invention.

In addition, the optical isomer can be separated by carrying out a known separation step such as a method using an optically active column or a fractional crystallization method in an appropriate step of the above production methods. In addition, an optically active substance can also be used as a starting material.

As used herein, a compound with stereochemistry (S, R) notation in its chemical structural formula means an optically active form, and when stereochemistry is not particularly indicated, the compound means a racemic form.

When a salt of compound (1) is to be obtained, if the salt of compound (1) can be obtained, the salt may be purified as it is, and if compound (1) is obtained in free form, the salt thereof may be formed by dissolving or suspending compound (1) in a suitable solvent and adding an acid or a base. In addition, compound (1) or a pharmaceutically acceptable salt thereof may exist in the form of a solvate with water or any of various solvents, and such a solvate is also encompassed by the present invention.

In the present invention, a derivative obtained by converting any one or two or more atoms of the compound represented by formula (1) into an isotope(s) is also encompassed by the compound represented by formula (1). For example, the “hydrogen atom” includes ¹H and ²H (D), and a deuterium conversion form obtained by converting any one or two or more 1H in the compound represented by formula (1) into ²H (D) is also encompassed by the compound represented by formula (1). In addition, for example, a conversion form into a radioactive isotope such as ¹¹C or ^{l8}F is also similarly encompassed by the compound represented by formula (1).

The compound of the present invention can be administered, directly or by being formulated into an appropriate dosage form, by oral administration or parenteral administration. Examples of the dosage form include, but are not limited to, a tablet, a capsule, a powder, a granule, a liquid, a suspension, an injection, a patch, and a cataplasm. The formulation is produced by a known method by using a pharmaceutically acceptable additive. As an additive, an excipient, a disintegrant, a binding agent, a plasticizer, a lubricant, a coating agent, a solubilizing agent, a dissolution aid, a thickening agent, a dispersing agent, a stabilizing agent, a sweetening agent, a flavoring agent, or the like can be used depending on the purpose. Specifically, examples thereof include lactose, mannitol, crystalline cellulose, low substituted hydroxypropylcellulose, corn starch, partially pregelatinized starch, carmellose calcium, croscarmellose sodium, hydroxypropylcellulose, hydroxypropylmethylcellulose, polyvinyl alcohol, magnesium stearate, sodium stearyl fumarate, polyethylene glycol, propylene glycol, titanium oxide, and talc.

The administration route of the compound of the present invention may be oral administration, parenteral administration, or rectal administration, and the daily dosage thereof varies depending on the type of the compound, the administration method, the symptom/age of the patient, and the like. For example, in the case of oral administration, usually about 0.01 to 1000 mg, further preferably about 0.1 to 500 mg, per kg of human or mammal body weight can be administered in one to several divided doses. In the case of parenteral administration such as intravenous injection, usually, for example, about 0.01 to 300 mg, further preferably about 1 to 100 mg, per kg of human or mammal body weight can be administered.

In addition, compound (1) of the present invention or a pharmaceutically acceptable salt thereof can be used, as a DYRK inhibitor, as a reagent for pathological imagery related to the above diseases or a reagent for a basic experiment or for research.

### EXAMPLES

Hereinafter, the present invention will be described more specifically with reference to Examples, Reference Examples, and Test Examples, but the present invention is not limited thereto at all. The compound names shown in the following Examples and Reference Examples do not necessarily follow the IUPAC nomenclature.

The following abbreviations may be used herein.
(Boc)₂O: di-tert-butyl dicarbonate
Boc: tert-butoxycarbonyl
Bn: benzyl
Cbz: benzyloxycarbonyl
Ns: 2-nitrobenzenesulfonyl
TBDPS: tert-butyldiphenylsilyl
Ac: acetyl
Ms: methanesulfonyl
DMSO: dimethyl sulfoxide
Rt: retention time
Et: ethyl
Teoc: 2-(trimethylsilyl)ethoxycarbonyl

Physicochemical data of each compound of the Examples and the Reference Examples were obtained with the following devices.

¹H-NMR: JEOL JNM-AL400; Brucker AVANCE 400 Spectrometer

LC/MS data of each compound of the Examples and the Reference Examples were obtained with the following devices.
Detection device: ACQUITY (R) SQ deteceter (Waters Corporation)
HPLC: ACQUITY UPLC (R)
SYSTEM Column: Waters ACQUITY UPLC (R) BEH C18 (1.7 um, 2.1 mm×30 mm)

The analysis conditions are as follows.

TABLE 1

| Method | Solvent | Gradient condition |
|---|---|---|
| Method A | Solvent A: | 0.0-1.3 min Linear |
| | 0.059% formic acid/water | gradient from B 2% to |
| | Solvent B: | 96% |
| | CH₃CN | |
| Method B | Solvent A: | 0.0-1.3 min Linear |
| | 0. 05% formic acid/water | gradient from B 10% to |
| | Solvent B: | 95% |
| | CH₃CN | |
| Method C | Solvent A: | 0.0-1.3 min Linear |
| | 0.05% formic acid/water | gradient from B 40% to |
| | Solvent B: | 95% |
| | CH₃CN | |

Flow rate: 0.8 mL/min;
Detection UV: 220 nm and 254 nm;
Temperature: 40° C.

Detection device: ACQUITY (R) QDa Detector (Waters Corporation)
HPLC: ACQUITY UPLC (R) H-Class PLUS
SYSTEM Column: Waters ACQUITY UPLC (R) C18 (1.7 um, 2.1 mm×30 mm)

The analysis conditions are as follows.

TABLE 2

| Method | Solvent | Gradient condition |
|---|---|---|
| Method D | Solvent A: | 0.0-1.3 min Linear |
| | 0.06% formic acid/water | gradient from B 2% to |
| | Solvent B: | 96% |
| | 0.06% formic acid/CH₃CN | |

Flow rate: 0.8 mL/min;
Detection UV: 220 nm and 254 nm;
Temperature: 40° C.

The compound names in the Reference Examples and the Examples were named by using ACD/Name (ACD/Labs 12.0, Advanced Chemistry Development Inc.).

### Reference Example 1

### tert-Butyl rac-[(1R,2R,3S)-2,3-dihydroxycyclopentyl]carbamate

### Reference Example 2

### tert-Butyl rac-[(1R,2R,3S)-2,3-dihydroxycyclopentyl]carbamate

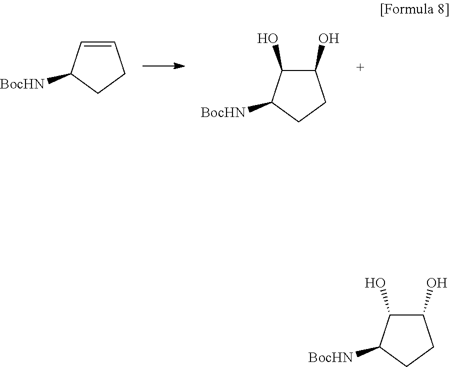 [SMILES: CC(C)(C)OC(=O)N[C@H]1C=CCC1 ; CC(C)(C)OC(=O)N[C@@H]1CC[C@H](O)[C@@H]1O ; CC(C)(C)OC(=O)N[C@@H]1CC[C@@H](O)[C@H]1O]

Potassium osmate(VI) dihydrate (536 mg) and 4-methylmorpholine-4-oxide monohydrate (4.99 g) were added to a solution (110 mL) of N-1-Boc-amino-2-cyclopentene (5.33 g) in acetone-water (10:1) at room temperature, and the resulting mixture was stirred at room temperature for 19 hours. A saturated sodium thiosulfate aqueous solution was added to the reaction mixture under ice cooling, and the resulting mixture was extracted with ethyl acetate. The organic layer was dried over sodium sulfate, filtered, and concentrated under reduced pressure. The residue was purified by silica gel column chromatography (hexane/ethyl acetate) to obtain the title compound (5.05 g) as a mixture at about 5:4.

¹H-NMR (CDCl₃) δ: 4.98 (1H, brs), 4.69 (1H, brs), 4.17-4.06 (2H, m), 4.00-3.82 (3H, m), 3.78-3.73 (1H, m), 2.34 (4H, brs), 2.28-2.17 (2H, m), 2.04-1.91 (2H, m), 1.88-1.56 (4H, m), 1.45-1.24 (2H, m), 51.43 (18H, s)

### Reference Example 3

### tert-Butyl rac-[(3aR,4R,6aS)-2,2-dimethyltetrahydro-2H,3aH-cyclopenta[d][1,3]dioxol-4-yl]methylcarbamate

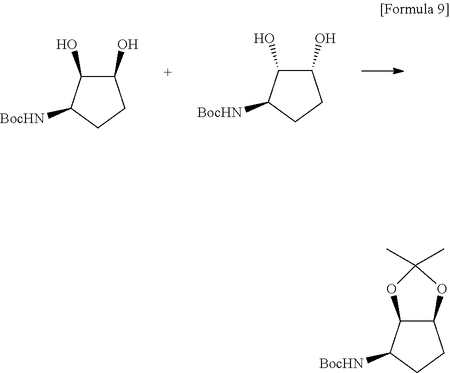 [SMILES: CC(C)(C)OC(=O)N[C@@H]1CC[C@H](O)[C@@H]1O ; CC(C)(C)OC(=O)N[C@@H]1CC[C@@H](O)[C@H]1O ; CC(C)(C)OC(=O)N[C@@H]1CC[C@@H]2OC(C)(C)O[C@@H]21]

Pyridinium p-toluenesulfonate (265 mg) was added to a 2,2-dimethoxypropane solution (90 mL) of a mixture of Reference Examples 1 and 2 (4.53 g) at room temperature, and the resulting mixture was stirred at room temperature for 4.5 hours. Saturated aqueous sodium bicarbonate was added to the reaction mixture under ice cooling, and the resulting mixture was extracted with ethyl acetate. The organic layer was dried over sodium sulfate, filtered, and concentrated under reduced pressure. The residue was purified by silica gel column chromatography (hexane/ethyl acetate) to obtain the title compound (2.71 g) as a mixture.

¹H-NMR (CDCl₃) δ: 4.98-4.92 (1H, m), 4.58 (1H, dd, J=5.2, 5.5 Hz), 4.41 (1H, dd, J=5.2, 5.5 Hz), 3.79-3.70 (1H, m), 1.88-1.81 (1H, m), 1.77 (1H, dd, J=5.5, 13.4 Hz), 1.55-1.36 (2H, m), 1.43 (3H, s), 1.43 (9H, s), 1.28 (3H s).

### Reference Example 4

### tert-Butyl rac-[(3aR,4R,6aS)-2,2-dimethyltetrahydro-2H,3aH-cyclopenta[d][1,3]dioxol-4-yl]methylcarbamate

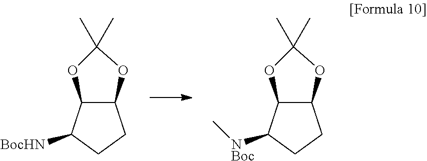 [SMILES: CC(C)(C)OC(=O)N[C@@H]1CC[C@@H]2OC(C)(C)O[C@@H]21 ; CN(C(=O)OC(C)(C)C)[C@@H]1CC[C@@H]2OC(C)(C)O[C@@H]21]

Sodium tert-butoxide (1.32 g) was added to a dimethylformamide solution (10 mL) of the compound of Reference Example 3 (2.71 g) under ice cooling, and the resulting mixture was stirred at room temperature for 45 minutes. Iodomethane (0.984 mL) and dimethylformamide (10 mL) were added to the reaction mixture under ice cooling, the resulting mixture was stirred at room temperature for 1.5 hours, then dimethylformamide (15 mL) was added, and the resulting mixture was allowed to stand overnight. The reaction mixture was stirred at room temperature for 4 hours, then sodium tert-butoxide (0.660 g) and iodomethane (0.492 mL) were added under ice cooling, and the resulting mixture was stirred at room temperature for 3.5 hours. Water was added to the reaction mixture, and the resulting mixture was extracted with ethyl acetate. The organic layer was washed twice with saturated brine, then dried over sodium sulfate, filtered, and concentrated under reduced pressure. The residue was purified by silica gel column chromatography (hexane/ethyl acetate) to obtain the title compound (2.59 g).

LC-MS [M+H]⁺/Rt (min): 272.2/1.037 (Method A)

### Reference Example 5

### tert-Butyl rac-[(1R,2R,3S)-2,3-dihydroxycyclopentyl]methylcarbamate

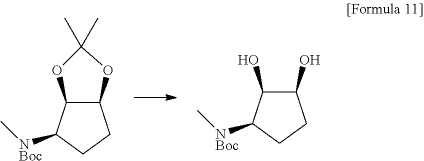 [SMILES: CN(C(=O)OC(C)(C)C)[C@@H]1CC[C@@H]2OC(C)(C)O[C@@H]21 ; CN(C(=O)OC(C)(C)C)[C@@H]1CC[C@H](O)[C@@H]1O]

A 2 M hydrogen chloride ethanol solution (11 mL) was added to the compound of Reference Example 4 (2.04 g), the resulting mixture was stirred at 100° C. for 15 minutes, then 6 M hydrochloric acid (5 mL) was added, and the resulting mixture was stirred at 100-C for 12 hours. Further, 6 M hydrochloric acid (5 mL) was added, and the resulting mixture was stirred at 120° C. for 10 hours, and then returned to room temperature and concentrated under reduced pressure. Triethylamine (2.62 mL) and Boc₂O (1.80 g) were added to a solution of the residue in methanol (20 mL), and the resulting mixture was stirred at room temperature for 1 hour. The reaction mixture was concentrated under reduced pressure, saturated aqueous sodium bicarbonate was added, and the resulting mixture was extracted with ethyl acetate. The organic layer was washed with saturated brine and dried over sodium sulfate, then filtered, and concentrated under reduced pressure. The residue was purified by silica gel column chromatography (hexane/ethyl acetate) to obtain the title compound (1.74 g).

LC-MS [M+H]⁺/Rt (min): 232.2/0.651 (Method A)

### Reference Example 6

### (1R,2S,3S)-3-[(tert-Butoxycarbonyl)(methyl)amino]cyclopentane-1,2-diyl rac-di-methanesulfonate

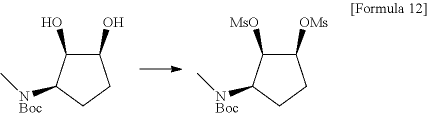 [SMILES: CN(C(=O)OC(C)(C)C)[C@@H]1CC[C@H](O)[C@@H]1O ; CN(C(=O)OC(C)(C)C)[C@@H]1CC[C@H](OS(C)(=O)=O)[C@@H]1OS(C)(=O)=O]

Methanesulfonyl chloride (1.50 mL) was added dropwise to a tetrahydrofuran solution (60 mL) of the compound of Reference Example 5 (1.49 g) and triethylamine (3.59 mL) under ice cooling, and then the resulting mixture was stirred at room temperature for 3 hours. Triethylamine (1.80 mL) and methanesulfonyl chloride (0.75 mL) were added under ice cooling, and the resulting mixture was stirred at room temperature overnight. Saturated brine and saturated aqueous sodium bicarbonate were added to the reaction mixture, and the resulting mixture was extracted with ethyl acetate. The organic layer was washed with saturated brine and dried over sodium sulfate, then filtered, and concentrated under reduced pressure. The residue was purified by silica gel column chromatography (hexane/ethyl acetate) to obtain the title compound (1.83 g).

LC-MS [M+H]⁺/Rt (min): 388.1/0.776 (Method B).

### Reference Examples 7 and 8

The compounds of Reference Examples 7 and 8 were obtained by using the corresponding raw material compounds according to the method described in Reference Example 6.

TABLE 3

| Reference | | |
|---|---|---|
| Example | Chemical Structure | Physical property data |
| 7 | 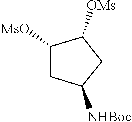 [SMILES: CC(C)(C)OC(=O)N[C@H]1C[C@H](OS(C)(=O)=O)[C@H](OS(C)(=O)=O)C1] | LC-MS [M + H]⁺/Rt (min): 374.1/0.682 (Method B) |
| 8 | 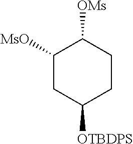 | LC-MS [M + H]⁺/Rt (min): 527.3/1.037 (Method C) |

### Reference Example 9

### tert-Butyl rac-[(1R,2S,3R)-2,3-diazidocyclopentyl]methylcarbamate

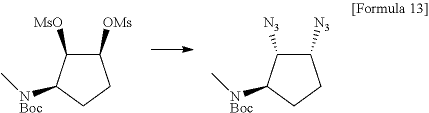 [SMILES: CN(C(=O)OC(C)(C)C)[C@@H]1CC[C@H](OS(C)(=O)=O)[C@@H]1OS(C)(=O)=O ; CN(C(=O)OC(C)(C)C)[C@@H]1CC[C@@H](N=[N+]=[N-])[C@H]1N=[N+]=[N-]]

Sodium azide (254 mg) was added to a dimethyl sulfoxide solution (4 mL) of the compound of Reference Example 6 (303 mg), and the resulting mixture was stirred at 100° C. for 30 hours. Water was added to the reaction mixture, and the resulting mixture was extracted with ethyl acetate. The organic layer was dried over sodium sulfate, filtered, and then concentrated under reduced pressure. The residue was purified by silica gel column chromatography (hexane/ethyl acetate) to obtain the title compound (49 mg).

LC-MS [M+H]⁺/Rt (min): 282.2/1.007 (Method B).

### Reference Examples 10 and 11

The compounds of Reference Examples 10 and 11 were obtained by using the corresponding raw material compounds according to the method described in Reference Example 9.

TABLE 4

| Reference | | |
|---|---|---|
| Example | Chemical Structure | Physical property data |
| 10 | 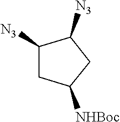 [SMILES: CC(C)(C)OC(=O)N[C@@H]1C[C@H](N=[N+]=[N-])[C@H](N=[N+]=[N-])C1] | LC-MS [M + H]⁺/Rt (min): 268.2/0.893 (Method B) |
| 11 | 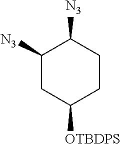 | LC-MS [M + H]⁺/Rt (min): 421.2/1.411 (Method C) |

### Reference Example 12

### tert-Butyl rac-[(1R,2R,3R)-2,3-diaminocyclopentyl]methylcarbamate

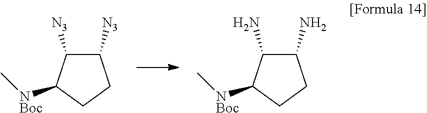 [SMILES: CN(C(=O)OC(C)(C)C)[C@@H]1CC[C@@H](N=[N+]=[N-])[C@H]1N=[N+]=[N-] ; CN(C(=O)OC(C)(C)C)[C@@H]1CC[C@@H](N)[C@H]1N]

Palladium hydroxide carbon (40 mg) was added to an ethanol solution (10 mL) of the compound of Reference Example 9 (344 mg), and the resulting mixture was stirred at room temperature for 7 hours in a hydrogen atmosphere. The reaction mixture was filtered through Celite and then concentrated under reduced pressure. The residue was purified by amino silica gel column chromatography (chloroform/methanol) to obtain the title compound (199 mg).

LC-MS [M+H]⁺/Rt (min): 230.2/0.470 (Method A).

### Reference Examples 13 to 15

The compounds of Reference Examples 13 to 15 were obtained by using the corresponding raw material compounds according to the method described in Reference Example 12.

TABLE 5

| Reference | | |
|---|---|---|
| Example | Chemical Structure | Physical property data |
| 13 | 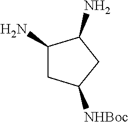 [SMILES: CC(C)(C)OC(=O)N[C@H]1C[C@@H](N)[C@@H](N)C1] | LC-MS [M + H]⁺/Rt (min): 216.1/0.170 (Method B) |
| 14 | 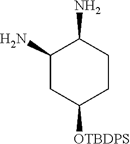 | LC-MS [M + H]⁺/Rt (min): 369.2/0.245 (Method C) |
| 15 | 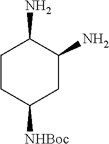 [SMILES: CC(C)(C)OC(=O)N[C@H]1CC[C@@H](N)[C@@H](N)C1] | LC-MS [M + H]⁺/Rt (min): 230.2/0.197 (Method B) |

### Reference Example 16

### rac-(1R,2R)-N¹-(4,4-Dimethylcyclohexyl)-1,2-diphenylethane-1,2-diamine

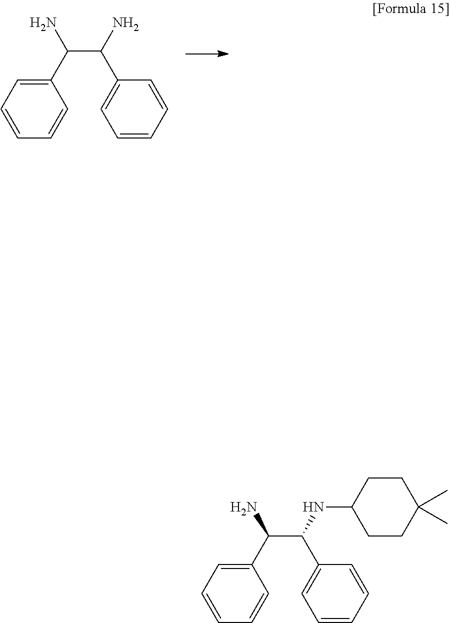 [SMILES: NC(c1ccccc1)C(N)c1ccccc1 ; CC1(C)CCC(N[C@H](c2ccccc2)[C@H](N)c2ccccc2)CC1]

4,4-Dimethylcyclohexanone (1.19 g) was added to a methanol solution (50 mL) of (±)-1,2-diphenylethylenediamine (1.0 g) at room temperature, and the resulting mixture was stirred for 1.5 hours. Sodium borohydride (711 mg) was added to the reaction mixture under ice cooling, and the resulting mixture was stirred at room temperature for 1 hour. Water was added to the reaction mixture, methanol was concentrated under reduced pressure, and then the resulting mixture was extracted with chloroform. The organic layer was dried over sodium sulfate, filtered, and then concentrated under reduced pressure. The residue was purified by silica gel column chromatography (hexane/ethyl acetate) to obtain the title compound (1.42 g).

LC-MS [M+H]⁺/Rt (min): 323.3/0.863 (Method A)

### Reference Example 17

### tert-Butyl rac-(1R,5R)-2-oxo-6-azabicyclo[3.1.0]hexane-6-carboxylate

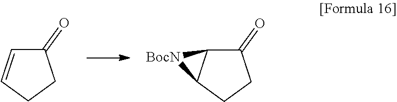 [SMILES: O=C1C=CCC1 ; CC(C)(C)OC(=O)N1[C@@H]2CCC(=O)[C@@H]21]

2-Cyclopenten-1-one (1.06 mL) was added dropwise to a chloroform suspension (45 mL) of the compound of Reference Example 16 (280 mg), N-Boc-O-tosylhydroxylamine (1.25 g), benzoic acid (530 mg), and sodium hydrogen carbonate (1.82 g) under ice cooling, and the resulting mixture was stirred at room temperature for 192 hours. Saturated aqueous sodium bicarbonate was added to the reaction mixture, and the resulting mixture was extracted with chloroform. The organic layer was dried over sodium sulfate, filtered, and then concentrated under reduced pressure. The residue was purified by silica gel column chromatography (chloroform/methanol) to obtain the title compound as a crude purified product (480 mg).

LC-MS [M+H]³⁰/Rt (min): 198.0/0.649 (Method B)

### Reference Example 18

### tert-Butyl rac-(1R,2R,5S)-2-(benzylamino)-6-azabicyclo[3.1.0]hexane-6-carboxylate

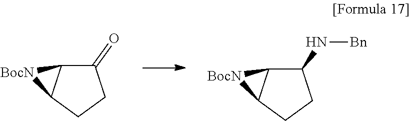 [SMILES: CC(C)(C)OC(=O)N1[C@@H]2CCC(=O)[C@@H]21 ; CC(C)(C)OC(=O)N1[C@H]2[C@@H](NCc3ccccc3)CC[C@H]21]

Benzylamine (0.399 mL) and sodium triacetoxyborohydride (1.03 g) were added to a solution of the compound of Reference Example 17 (480 mg) in tetrahydrofuran (8 mL) under ice cooling, and the resulting mixture was stirred under ice cooling for 0.5 hours and then stirred at room temperature overnight. Saturated aqueous sodium bicarbonate was added to the reaction mixture, and the resulting mixture was extracted with ethyl acetate. The organic layer was dried over sodium sulfate, filtered, and then concentrated under reduced pressure. The residue was purified by silica gel column chromatography (chloroform/methanol) to obtain the title compound (610 mg).

LC-MS [M+H]⁺/Rt (min): 289.2/0.529 (Method B)

### Reference Example 19

### tert-Butyl rac-(1R,2S,5R)-2-{benzyl[(benzyloxy)carbonyl]amino}-6-azabicyclo[3.1.0]hexane-6-carboxylate

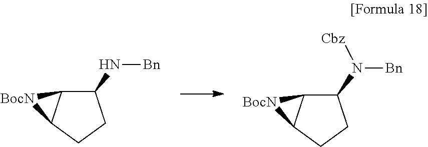 [SMILES: CC(C)(C)OC(=O)N1[C@H]2[C@@H](NCc3ccccc3)CC[C@H]21 ; CC(C)(C)OC(=O)N1[C@@H]2[C@H]1CC[C@@H]2N(Cc1ccccc1)C(=O)OCc1ccccc1]

Triethylamine (0.671 mL) and benzyl chloroformate (0.339 mL) were added to a solution of the compound of Reference Example 18 (347 mg) in tetrahydrofuran (6 mL) at room temperature, and the resulting mixture was stirred overnight. Triethylamine (0.671 mL) and benzyl chloroformate (0.678 mL) were further added to the reaction mixture under ice cooling, and the resulting mixture was stirred at room temperature for 8 hours and then allowed to stand overnight. Saturated aqueous sodium bicarbonate was added to the reaction mixture, and the resulting mixture was extracted with ethyl acetate. The organic layer was washed with saturated brine, and then dried over sodium sulfate, filtered, and concentrated under reduced pressure. The residue was purified by silica gel column chromatography (hexane/ethyl acetate) to obtain the title compound (403 mg).

LC-MS [M+H]⁺/Rt (min): 423.3/1.214 (Method B)

### Reference Example 20

### (1R,2R,3R)-3-{Benzyl[(benzyloxy)carbonyl]amino}-2-[(tert-butoxycarbonyl)amino]cyclopentyl rac-acetate

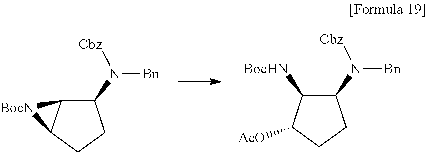 [SMILES: CC(C)(C)OC(=O)N1[C@@H]2[C@H]1CC[C@@H]2N(Cc1ccccc1)C(=O)OCc1ccccc1 ; CC(=O)O[C@H]1CC[C@H](N(Cc2ccccc2)C(=O)OCc2ccccc2)[C@@H]1NC(=O)OC(C)(C)C]

Acetic acid (19 mL) was added to the compound of Reference Example 19 (403 mg), and the resulting mixture was stirred for 2 hours and then allowed to stand overnight. Saturated aqueous sodium bicarbonate was added to the reaction mixture, and the resulting mixture was extracted with ethyl acetate. The organic layer was washed with saturated brine, and then dried over sodium sulfate, filtered, and concentrated under reduced pressure. The residue was purified by silica gel column chromatography (hexane/ethyl acetate) to obtain the title compound (392 mg).

LC-MS [M+H]⁺/Rt (min): 483.3/1.179 (Method B)

### Reference Example 21

### (1R,2R,3R)-3-Amino-2-[(tert-butoxycarbonyl)amino]cyclopentyl rac-acetate

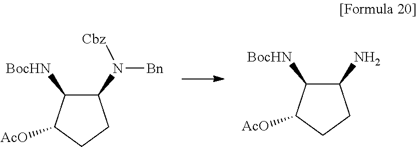 [SMILES: CC(=O)O[C@H]1CC[C@H](N(Cc2ccccc2)C(=O)OCc2ccccc2)[C@@H]1NC(=O)OC(C)(C)C ; CC(=O)O[C@H]1CC[C@H](N)[C@@H]1NC(=O)OC(C)(C)C]

Palladium hydroxide carbon (144 mg) was added to an ethanol solution (50 mL) of the compound of Reference Example 20 (2.88 g), and the resulting mixture was stirred at room temperature for 6 hours in a hydrogen atmosphere. The reaction mixture was filtered through Celite and then concentrated under reduced pressure to obtain the title compound (1.58 g).

LC-MS [M+H]⁺/Rt (min): 259.2/0.387 (Method B)

### Reference Example 22

### (1R,2R,3R)-2,3-Diaminocyclopentyl rac-acetate

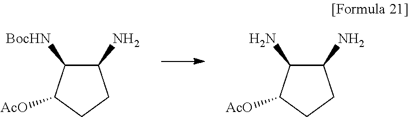 [SMILES: CC(=O)O[C@H]1CC[C@H](N)[C@@H]1NC(=O)OC(C)(C)C ; CC(=O)O[C@H]1CC[C@H](N)[C@@H]1N]

A 4 M hydrogen chloride ethyl acetate solution (20 mL) was added to a suspension of the compound of Reference Example 21 (1.58 g) in ethyl acetate (5 mL) at room temperature, and the resulting mixture was stirred for 2 hours. The reaction mixture was concentrated under reduced pressure to obtain a hydrochloride of the title compound (1.81 g).

LC-MS [M+H]⁺/Rt (min): 159.0/0.125 (Method B)

### Reference Example 23

### (1R,2R,3R)-2-Amino-3-{[(2,3-dihydro-1-benzofuran-4-yl)carbamothioyl]amino}cyclopentyl rac-acetate

### Reference Example 24

### (1R,2R,3R)-3-Amino-2-{[(2,3-dihydro-1-benzofuran-4-yl) carbamothioyl]amino}cyclopentyl rac-acetate

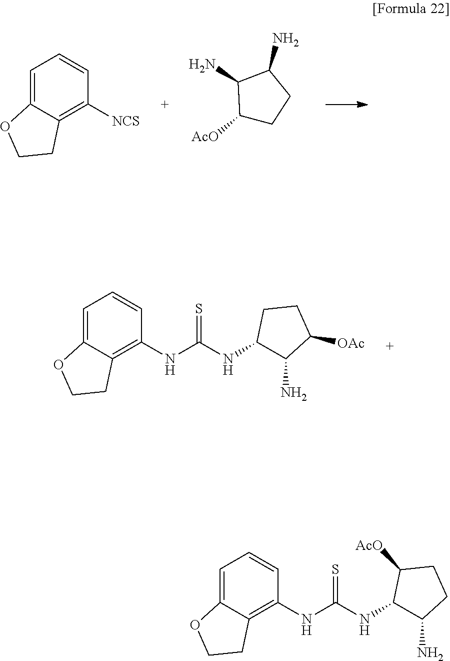

4-Isothiocyanato-2,3-dihydrobenzofuran (635 mg) was added to a chloroform solution (20 mL) of the compound of Reference Example 22 (1.81 g) and triethylamine (4.16 mL) under ice cooling, and the resulting mixture was stirred under ice cooling for 1 hour and then allowed to stand overnight. Chloroform (20 mL) was added at room temperature, and triethylamine (4.16 mL) and 4-isothiocyanato-2,3-dihydrobenzofuran (53 mg) were added under ice cooling. The resulting mixture was stirred for 2.5 hours under ice cooling, then 4-isothiocyanato-2,3-dihydrobenzofuran (53 mg) was further added, and the resulting mixture was stirred for 3 hours. 4-Isothiocyanato-2,3-dihydrobenzofuran (53 mg) was further added under ice cooling, the resulting mixture was stirred for 1 hour, then saturated aqueous sodium bicarbonate was added to the reaction mixture, and the resulting mixture was extracted with chloroform. The organic layer was dried over sodium sulfate, then filtered, and concentrated under reduced pressure. The residue was purified by silica gel column chromatography (chloroform/methanol) to obtain the title compound (Reference Example 23: 745 mg, Reference Example 24: 500 mg).

Reference Example 23: LC-MS [M+H]⁺/Rt (min): 336.1/0.515 (Method B)

Reference Example 24: LC-MS [M+H]⁺/Rt (min): 336.1/0.478 (Method B)

### Reference Examples 25 to 31

The compounds of Reference Examples 25 to 31 were obtained by using the corresponding raw material compounds according to the method described in Reference Examples 23 and 24.

TABLE 6

| Reference | | |
|---|---|---|
| Example | Chemical Structure | Physical property data |
| 25 | 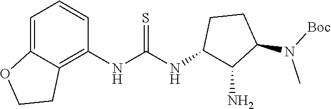 [SMILES: CN(C(=O)OC(C)(C)C)[C@@H]1CC[C@@H](NC(=S)Nc2cccc3c2CCO3)[C@H]1N] | LC-MS [M + H]⁺/Rt (min): 407.4/0.775 (Method A) |
| 26 | 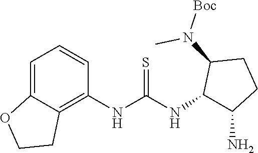 [SMILES: CN(C(=O)OC(C)(C)C)[C@H]1CC[C@H](N)[C@@H]1NC(=S)Nc1cccc2c1CCO2] | LC-MS [M + H]⁺/Rt (min): 407.4/0.723 (Method A) |
| 27 | 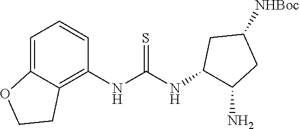 [SMILES: CC(C)(C)OC(=O)N[C@@H]1C[C@H](N)[C@H](NC(=S)Nc2cccc3c2CCO3)C1] | LC-MS [M + H]⁺/Rt (min): 393.2/0.594 (Method B) |
| 28 | 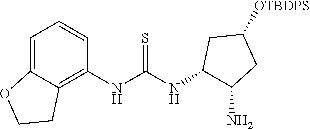 | LC-MS [M + H]⁺/Rt (min): 532.4/0.902 (Method B) |
| 29 | 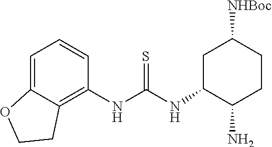 [SMILES: CC(C)(C)OC(=O)N[C@@H]1CC[C@H](N)[C@H](NC(=S)Nc2cccc3c2CCO3)C1] | LC-MS [M + H]⁺/Rt (min): 407.2/0.581 (Method B) |
| 30 | 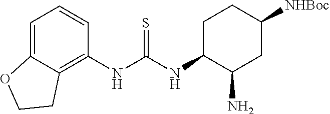 [SMILES: CC(C)(C)OC(=O)N[C@@H]1CC[C@H](NC(=S)Nc2cccc3c2CCO3)[C@H](N)C1] | LC-MS [M + H]⁺/Rt (min): 407.2/0.581 (Method B) |
| 31 | 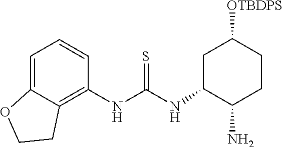 | LC-MS [M + H]⁺/Rt (min): 546.3/1.130 (Method B) |

### Reference Example 32

### (1R,2R,3R)-3-Amino-2-[(7,8-dihydrofuro[3,2-e][1,3]benzothiazol 2-yl)amino]cyclopentyl rac-acetate

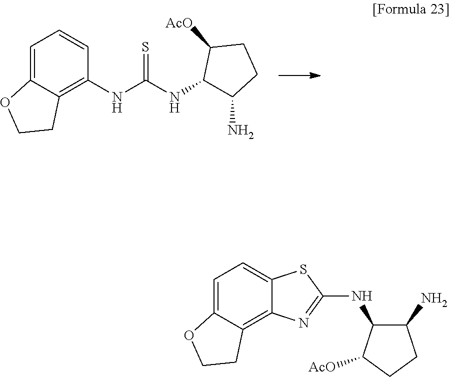 [SMILES: CC(=O)O[C@H]1CC[C@H](N)[C@@H]1NC(=S)Nc1cccc2c1CCO2 ; CC(=O)O[C@H]1CC[C@H](N)[C@@H]1Nc1nc2c3c(ccc2s1)OCC3]

Benzyltrimethylammonium tribromide (558 mg) was added to a chloroform solution (15 mL) of the compound of Reference Example 24 (500 mg), and the resulting mixture was stirred overnight. Saturated aqueous sodium bicarbonate was added to the reaction mixture, and the resulting mixture was extracted with chloroform/ethanol (3/1). The organic layer was dried over sodium sulfate, filtered, and then concentrated under reduced pressure. The residue was purified by silica gel column chromatography (chloroform/methanol) to obtain the title compound (450 mg).

LC-MS [M+H]⁺/Rt (min): 334.1/0.508 (Method B)

### Reference Examples 33 to 40

The compounds of Reference Examples 33 to 40 were obtained by using the corresponding raw material compounds according to the method described in Reference Example 32.

TABLE 7

| Reference | | |
|---|---|---|
| Example | Chemical Structure | Physical property data |
| 33 | 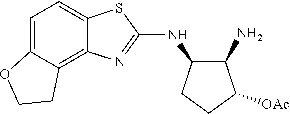 [SMILES: CC(=O)O[C@@H]1CC[C@@H](Nc2nc3c4c(ccc3s2)OCC4)[C@H]1N] | LC-MS [M + H]⁺/Rt (min): 334.1/0.521 (Method B) |
| 34 | 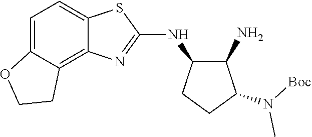 [SMILES: CN(C(=O)OC(C)(C)C)[C@@H]1CC[C@@H](Nc2nc3c4c(ccc3s2)OCC4)[C@H]1N] | LC-MS [M + H]⁺/Rt (min): 405.3/0.729 (Method A) |
| 35 | 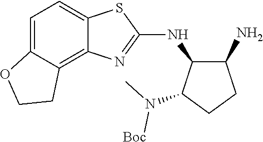 [SMILES: CN(C(=O)OC(C)(C)C)[C@H]1CC[C@H](N)[C@@H]1Nc1nc2c3c(ccc2s1)OCC3] | LC-MS [M + H]⁺/Rt (min): 405.4/0.745 (Method A) |
| 36 | 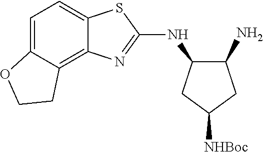 [SMILES: CC(C)(C)OC(=O)N[C@@H]1C[C@H](N)[C@H](Nc2nc3c4c(ccc3s2)OCC4)C1] | LC-MS [M + H]⁺/Rt (min): 391.3/0.735 (Method A) |
| 37 | 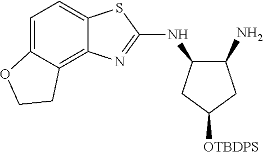 | LC-MS [M + H]⁺/Rt (min): 530.3/1.049 (Method B) |
| 38 | 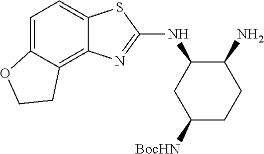 [SMILES: CC(C)(C)OC(=O)N[C@@H]1CC[C@H](N)[C@H](Nc2nc3c4c(ccc3s2)OCC4)C1] | LC-MS [M + H]⁺/Rt (min): 405.2/0.572 (Method B) |
| 39 | 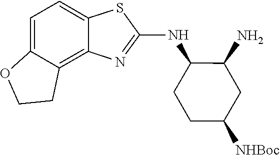 [SMILES: CC(C)(C)OC(=O)N[C@H]1CC[C@@H](Nc2nc3c4c(ccc3s2)OCC4)[C@@H](N)C1] | LC-MS [M + H]⁺/Rt (min): 405.2/0.572 (Method B) |
| 40 | 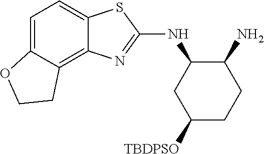 | LC-MS [M + H]⁺/Rt (min): 544.3/1.123 (Method B) |

### Reference Examples 41 and 42

The compounds of Reference Examples 41 and 42 were obtained by using the corresponding raw material compounds according to the method described in Example 1.

TABLE 8

| Reference | | |
|---|---|---|
| Example | Chemical Structure | Physical property data |
| 41 | 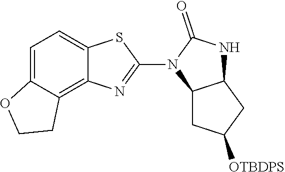 | LC-MS [M + H]⁺/Rt (min): 556.3/1.298 (Method B) |
| 42 | 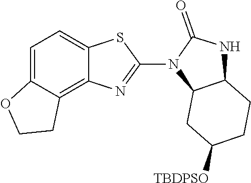 | LLC-MS [M + H]⁺/Rt (min): 570.3/1.391 (Method B) |

### Reference Example 43

### tert-Butyl rac-(3aR,4R,6aR)-3-(7,8-dihydrofuro[3,2-e][1,3]benzothiazol 2-yl)-4-hydroxy-2-oxohexahydrocyclopenta[d]imidazole 1 (2H)-carboxylate

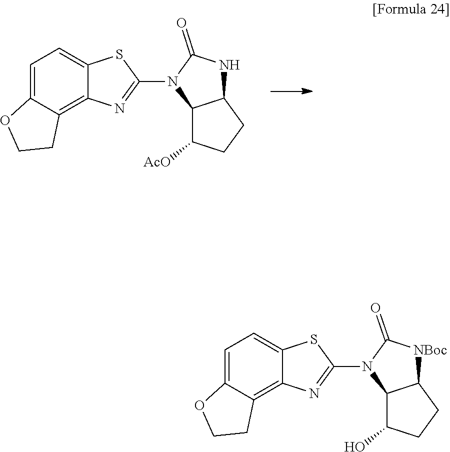 [SMILES: CC(=O)O[C@H]1CC[C@@H]2NC(=O)N(c3nc4c5c(ccc4s3)OCC5)[C@@H]21 ; CC(C)(C)OC(=O)N1C(=O)N(c2nc3c4c(ccc3s2)OCC4)[C@@H]2[C@@H](O)CC[C@@H]21]

Triethylamine (0.278 mL), (Boc)₂O (164 mg), and dimethylaminopyridine (one piece) were added to a chloroform/acetonitrile solution (5 mL/1 mL) of the compound of Example 7 (180 mg), and the resulting mixture was allowed to stand at room temperature overnight. The reaction mixture was concentrated under reduced pressure, and the residue was purified by silica gel column chromatography (chloroform/methanol). Potassium carbonate (104 mg) was added to a methanol/tetrahydrofuran solution (6 mL/10 mL) of the resulting solid (265 mg), and the resulting mixture was stirred at room temperature for 1 hour. Chloroform, water, and saturated aqueous sodium bicarbonate were added to the reaction mixture, and the resulting mixture was extracted with chloroform/ethanol (3/1). The organic layer was dried over sodium sulfate, filtered, and then concentrated under reduced pressure. Triethylamine (0.105 mL), (Boc)₂O (109 mg), and dimethylaminopyridine (3.1 mg) were added to a THF solution (6 mL) of the residue, and the resulting mixture was stirred at room temperature for 1 hour. Saturated aqueous sodium bicarbonate was added to the reaction mixture, and the resulting mixture was extracted with chloroform/ethanol (3/1). The organic layer was dried over sodium sulfate, filtered, and then concentrated under reduced pressure. The residue was purified by silica gel column chromatography (chloroform/methanol) to obtain the title compound (212 mg).

LC-MS [M+H]⁺/Rt (min): 418.2/0.942 (Method B)

### Reference Example 44

tert-Butyl rac-(3aR,6aR)-3-(7,8-dihydrofuro[3,2-e][1,3]benzothiazol 2-yl)-2,4-dioxohexahydrocyclopenta[d]imidazole 1(2H)-carboxylate

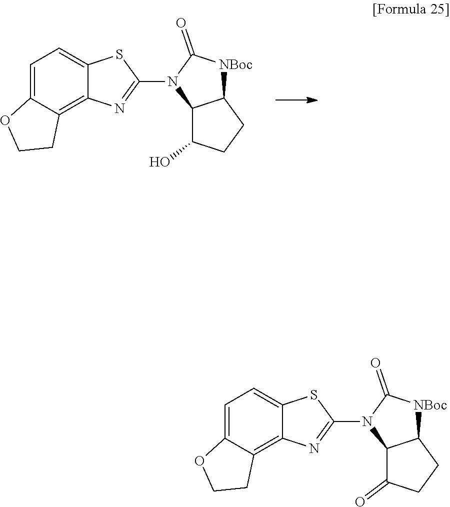 [SMILES: CC(C)(C)OC(=O)N1C(=O)N(c2nc3c4c(ccc3s2)OCC4)[C@@H]2[C@@H](O)CC[C@@H]21 ; CC(C)(C)OC(=O)N1C(=O)N(c2nc3c4c(ccc3s2)OCC4)[C@@H]2C(=O)CC[C@@H]21]

The Dess-Martin reagent (551 mg) was added to a chloroform solution (10 mL) of the compound of Reference Example 43 (212 mg), and the resulting mixture was stirred for 2 hours. Saturated aqueous sodium bicarbonate and a saturated sodium thiosulfate aqueous solution were added to the reaction mixture, and the resulting mixture was extracted with chloroform/ethanol (3/1). The organic layer was dried over sodium sulfate, filtered, and then concentrated under reduced pressure. The residue was purified by silica gel column chromatography (chloroform/methanol) to obtain the title compound (200 mg).

LC-MS [M+H]⁺/Rt (min): 416.1/0.920 (Method B)

### Reference Example 45

The compound of Reference Example 45 was obtained by using the corresponding raw material compounds according to the method described in Reference Example 44.

TABLE 9

| Reference | | |
|---|---|---|
| Example | Chemical Structure | Physical property data |
| 45 | 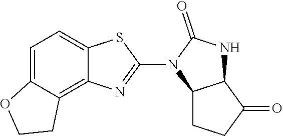 [SMILES: O=C1CC[C@@H]2[C@H]1NC(=O)N2c1nc2c3c(ccc2s1)OCC3] | LC-MS [M + H]⁺/Rt (min): 316.1/0.672 (Method B) |

### Reference Example 46

### rac-2-[(3aR,5R,6aS)-1-(7,8-Dihydrofuro[3,2-e][1,3]benzothiazol 2-yl)-2-oxooctahydrocyclopenta[d]imidazol 5-yl]-1H-isoindole-1,3 (2H)-dione

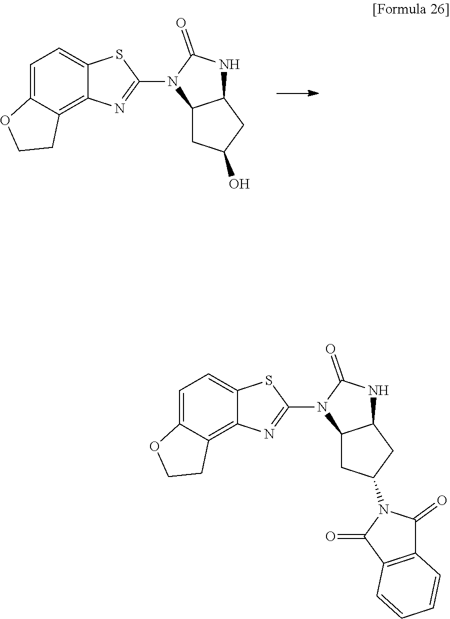 [SMILES: O=C1N[C@H]2C[C@@H](O)C[C@H]2N1c1nc2c3c(ccc2s1)OCC3 ; O=C1c2ccccc2C(=O)N1[C@H]1C[C@@H]2NC(=O)N(c3nc4c5c(ccc4s3)OCC5)[C@@H]2C1]

Phthalimide (19 mg), triphenylphosphine (39 mg), and diisopropyl azodicarboxylate (0.029 mL) were added to a dimethylformamide solution (1 mL) of the compound of Example 8 (32 mg), and the resulting mixture was stirred at 50° C. for 4 hours. Water was added to the reaction mixture, and the resulting mixture was extracted with chloroform. The organic layer was dried over sodium sulfate, filtered, and then concentrated under reduced pressure. The residue was purified by silica gel column chromatography (hexane/ethyl acetate) to obtain the title compound (30 mg).

LC-MS [M+H]⁺/Rt (min): 447.1/0.873 (Method B)

### Reference Example 47

The compound of Reference Example 47 was obtained by using the corresponding raw material compounds according to the method described in Reference Example 46.

TABLE 10

| Reference | | |
|---|---|---|
| Example | Chemical Structure | Physical property data |
| 47 | 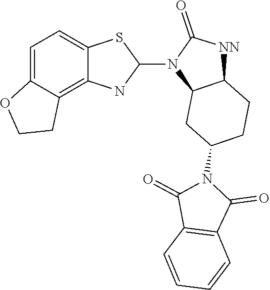 | LC-MS [M + H]⁺/Rt (min): 461.2/0.877 (Method B) |

### Reference Example 48

rac-(3aR,5R,6aS)-5-{[tert-Butyl(diphenyl)silyl]oxy)-1-(7,8-dihydrofuro[3,2-e][1,3]benzothiazol 2-yl)-3-(2-nitrobenzene-1-sulfonyl)hexahydrocyclopenta[d]imidazol 2 (1H)-one

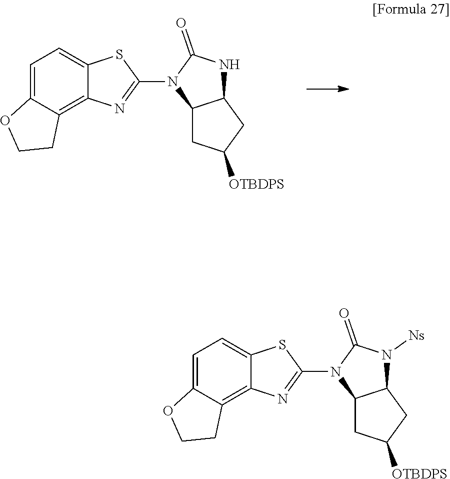

Sodium hydride (36 mg) and 2-nitrobenzenesulfonyl chloride (130 mg) were added to a tetrahydrofuran solution (2 mL) of the compound of Reference Example 41 (110 mg), and the resulting mixture was stirred at 60° C. for 2 hours. Water was added to the reaction mixture, and the resulting mixture was extracted with ethyl acetate. The organic layer was dried over sodium sulfate, filtered, and then concentrated under reduced pressure. The residue was purified by silica gel column chromatography (hexane/ethyl acetate) to obtain the title compound (100 mg).

LC-MS [M+H]⁺/Rt (min): 741.2/1.338 (Method C)

### Reference Example 49

The compound of Reference Example 49 was obtained by using the corresponding raw material compounds according to the method described in Example 8.

TABLE 11

| Reference | | Physical |
|---|---|---|
| Example | Chemical Structure | property data |
| 49 | 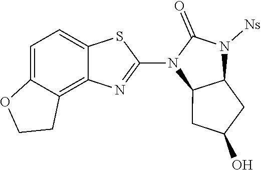 | LC-MS [M + H]⁺/ Rt (min): 503.2/0.545 (Method C) |

### Reference Example 50

### rac-(3aR,5R,6aS)-1-(7,8-dihydrofuro[3,2-e][1,3]benzothiazol 2-yl)-5-methoxy-3-(2-nitrobenzene-1-sulfonyl)hexahydrocyclopenta[d]imidazol 2(1H)-one

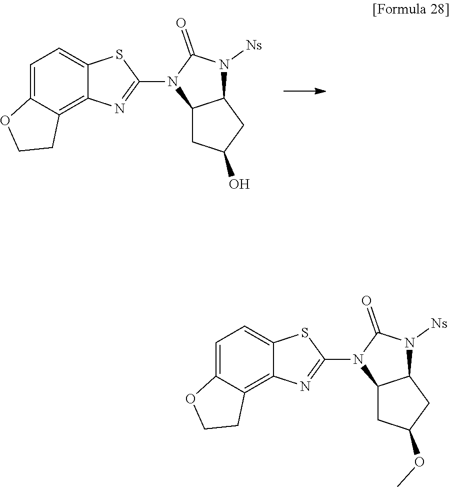

Sodium hydride (10 mg) and iodomethane (0.015 mL) were added to a dimethylformamide solution (1 mL) of the compound of Reference Example 49 (42 mg), and the resulting mixture was stirred at room temperature for 1 hour. Water was added to the reaction mixture, and the resulting mixture was extracted with ethyl acetate. The organic layer was dried over sodium sulfate, filtered, and then concentrated under reduced pressure. The residue was purified by silica gel column chromatography (hexane/ethyl acetate) to obtain the title compound (10 mg).

LC-MS [M+H]⁺/Rt (min): 517.2/0.598 (Method C)

### Reference Example 51

The compound of Reference Example 51 was obtained by using the corresponding raw material compounds according to the method described in Reference Example 18.

TABLE 12

| Reference | | |
|---|---|---|
| Example | Chemical Structure | Physical property data |
| 51 | 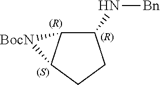 | LC-MS [M + H]⁺/Rt (min): 289.3/0.708 (Method A). |

### Reference Example 52

The compound of Reference Example 52 was obtained by using the corresponding raw material compounds according to the method described in Reference Example 19.

TABLE 13

| Reference | | |
|---|---|---|
| Example | Chemical Structure | Physical property data |
| 52 | 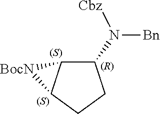 | LC-MS [M + H]⁺/Rt (min): 423.3/1.215 (Method B). |

### Reference Example 53

### tert-Butyl [(1R,2R,5R)-2-azido-5-{benzyl[(benzyloxy) carbonyl]amino}cyclopentyl]carbamate

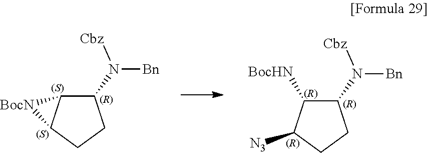

Trimethylsilyl azide (0.196 mL) and a boron trifluoride diethyl ether complex (0.013 mL) were added to a chloroform solution (10 mL) of the compound of Reference Example 52 (423 mg) under ice cooling, and the resulting mixture was stirred under ice cooling for 1.5 hours and at room temperature for 1.5 hours. The boron trifluoride diethyl ether complex (0.305 mL) was added under ice cooling, and the resulting mixture was stirred at room temperature overnight. Saturated aqueous sodium bicarbonate was added to the reaction mixture, and the resulting mixture was extracted with chloroform. The organic layer was dried over sodium sulfate, then filtered, and concentrated under reduced pressure. Triethylamine (0.209 mL) and (Boc)₂O (218 mg) were added to a chloroform solution (8 mL) of the residue, and the resulting mixture was stirred at room temperature overnight. Saturated aqueous sodium bicarbonate and saturated brine were added to the reaction mixture, and the resulting mixture was extracted with ethyl acetate. The organic layer was dried over sodium sulfate, then filtered, and concentrated under reduced pressure. The residue was purified by silica gel column chromatography (hexane/ethyl acetate) to obtain the title compound (212 mg).

LC-MS [M+H]⁺/Rt (min): 466.4/1.280 (Method A)

### Reference Example 54

### tert-Butyl [(1S,2R,5R)-2-amino-5-{benzyl[(benzyloxy)carbonyl]amino}cyclopentyl]carbamate

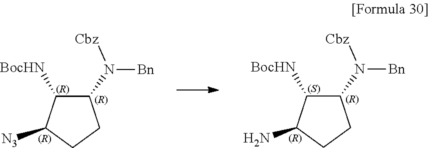

Triphenylphosphine (125 mg) was added to a tetrahydrofuran/water solution (5 mL/0.5 mL) of the compound of Reference Example 53 (212 mg), and the resulting mixture was stirred at 60° C. for 5 hours. Water (0.5 mL) was added, and the resulting mixture was stirred at 70° C. for 3 hours. The reaction mixture was cooled to room temperature and concentrated under reduced pressure. The residue was purified by silica gel column chromatography (chloroform/methanol) to obtain the title compound (202 mg).

LC-MS [M+H]⁺/Rt (min): 440.4/0.820 (Method B)

### Reference Example 55

### Benzyl benzyl[(1R,2S,3R)-2-[(tert-butoxycarbonyl)amino]-3-(2,2,2-trifluoroacetamido)cyclopentyl]carbamate

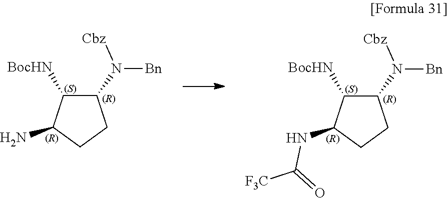

Trifluoroacetic anhydride (0.069 mL) was added to a chloroform solution (4 mL) of the compound of Reference Example 54 (202 mg) and diisopropylethylamine (0.119 mL) under ice cooling, and the resulting mixture was stirred under ice cooling for 1.5 hours and then stirred at room temperature for 2 hours. Diisopropylethylamine (0.238 mL) and trifluoroacetic anhydride (0.138 mL) were added at room temperature, and the resulting mixture was stirred for 1.5 hours. Saturated aqueous sodium bicarbonate was added to the reaction mixture, and the resulting mixture was extracted with chloroform. The organic layer was dried over sodium sulfate, then filtered, and concentrated under reduced pressure. The residue was purified by silica gel column chromatography (hexane/ethyl acetate) to obtain the title compound (224 mg).

LC-MS [M+H]⁺/Rt (min): 536.4/1.161 (Method B)

### Reference Example 56

The compound of Reference Example 56 was obtained by using the corresponding raw material compounds according to the method described in Reference Example 21.

TABLE 14

| Ref- | | |
|---|---|---|
| erence | | |
| Example | Chemical Structure | Physical property data |
| 56 | 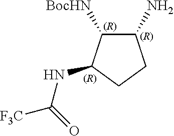 | LC-MS [M + H]⁺/Rt (min): 312.2/0.428 (Method B) |

### Reference Example 57

### 5-Bromo-4-isothiocyanato-2,3-dihydrobenzofuran

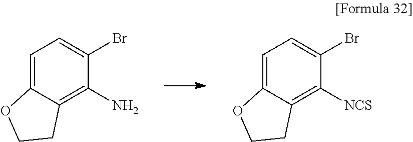

5-Bromo-2,3-dihydrobenzofuranbenzodioxan-4-amine (825 mg) was added to a chloroform solution (8.5 mL) of 1,1′-thiocarbonyldi-2(1H)-pyridone (940 mg) at room temperature, and the resulting mixture was stirred at room temperature for 5 hours. Water was added to the reaction mixture, and the resulting mixture was extracted with chloroform. The organic layer was dried over sodium sulfate, filtered, and then concentrated under reduced pressure. The residue was purified by silica gel column chromatography (hexane/ethyl acetate) to obtain the title compound (928 mg).

¹H-NMR (CDCl₃) δ: 7.25 (1H, d, J=8.5 Hz), 6.55 (1H, d, J=8.5 Hz), 4.52 (2H, t, J=8.8 Hz), 3.30 (2H, t, J=8.8 Hz).

### Reference Example 58

### tert-Butyl [(1S,2R,5R)-2-[(7,8-dihydrofuro[3,2-e][1,3]benzothiazol 2-yl)amino)-5-(2,2,2-trifluoroacetamido)cyclopentyl]carbamate

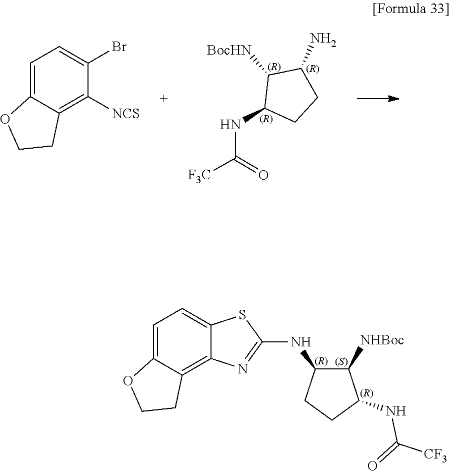

The compound of Reference Example 57 (96 mg), potassium carbonate (104 mg), 1,10-phenanthroline (6.8 mg), and copper iodide (3.6 mg) were added to a tetrahydrofuran solution (3 mL) of Reference Example 56 (117 mg) at room temperature, and the resulting mixture was stirred at room temperature overnight. The reaction mixture was diluted with ethyl acetate and filtered through Celite. Saturated aqueous sodium bicarbonate was added to the filtrate, and the resulting mixture was extracted with ethyl acetate. The organic layer was washed with saturated brine, and then dried over sodium sulfate, filtered, and concentrated under reduced pressure. Methanol was added to the residue, the resulting mixture was stirred, and the solid was collected by filtration and then dried to obtain the title compound (133 mg).

LC-MS [M+H]⁺/Rt (min): 487.3/0.882 (Method B)

### Reference Example 59

The compound of Reference Example 59 was obtained by using the corresponding raw material compounds according to the method described in Reference Example 22.

TABLE 15

| Reference | | |
|---|---|---|
| Example | Chemical Structure | Physical property data |
| 59 | 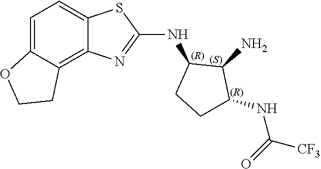 | LC-MS [M + H]⁺/Rt (min): 387.2/0.536 (Method B) |

### Reference Example 60

The compound of Reference Example 60 was obtained by using the corresponding raw material compounds according to the method described in Reference Example 18.

TABLE 16

| Reference | | |
|---|---|---|
| Example | Chemical Structure | Physical property data |
| 60 | 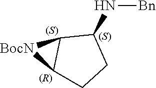 | LC-MS [M + H]⁺/Rt (min): 289.2/0.637 (Method A) |

### Reference Example 61

The compound of Reference Example 61 was obtained by using the corresponding raw material compounds according to the method described in Reference Example 19.

TABLE 17

| Reference | | |
|---|---|---|
| Example | Chemical Structure | Physical property data |
| 61 | 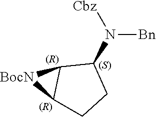 | LC-MS [M + H]⁺/Rt (min): 423.3/1.258 (Method A) |

### Reference Example 62

The compound of Reference Example 62 was obtained by using the corresponding raw material compounds according to the method described in Reference Example 53.

TABLE 18

| Reference | | |
|---|---|---|
| Example | Chemical Structure | Physical property data |
| 62 | 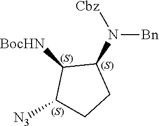 | LC-MS [M + H]⁺/Rt (min): 466.4/1.280 (Method A) |

### Reference Example 63

The compound of Reference Example 63 was obtained by using the corresponding raw material compounds according to the method described in Reference Example 54.

TABLE 19

| Reference | | |
|---|---|---|
| Example | Chemical Structure | Physical property data |
| 63 | 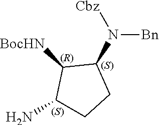 | LC-MS [M + H]⁺/Rt (min): 440.4/1.020 (Method A) |

### Reference Example 64

### tert-Butyl 2-(trimethylsilyl)ethyl [(1S,2R,3S)-3-{benzyl[(benzyloxy)carbonyl]amino}cyclopentane-1,2-diyl]biscarbamate

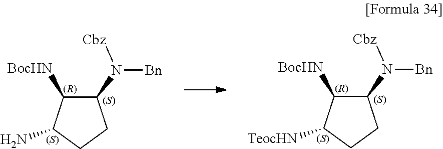

N-[2-(Trimethylsilyl)ethoxycarbonyloxy]succinimide (293 mg) was added to a tetrahydrofuran solution (12 mL) of the compound of Reference Example 63 (414 mg) and triethylamine (0.196 mL) at room temperature, and the resulting mixture was stirred at room temperature overnight. Saturated aqueous sodium bicarbonate was added to the reaction mixture, and the resulting mixture was extracted with ethyl acetate. The organic layer was dried over sodium sulfate, then filtered, and concentrated under reduced pressure. The residue was purified by silica gel column chromatography (hexane/ethyl acetate) to obtain the title compound (528 mg).

LC-MS [M+H]⁺/Rt (min): 584.4/1.372 (Method A)

### Reference Example 65

The compound of Reference Example 65 was obtained by using the corresponding raw material compounds according to the method described in Reference Example 21.

TABLE 20

| Reference | | |
|---|---|---|
| Example | Chemical Structure | Physical property data |
| 65 | 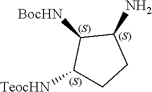 | LC-MS [M + H]⁺/Rt (min): 360.4/0.809 (Method A) |

### Reference Example 66

The compound of Reference Example 66 was obtained by using the corresponding raw material compounds according to the method described in Reference Example 19.

TABLE 21

| Reference | | |
|---|---|---|
| Example | Chemical Structure | Physical property data |
| 66 | 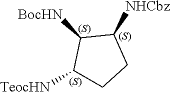 | LC-MS [M + H]⁺/Rt (min): 494.4/1.196 (Method A) |

### Reference Example 67

### Benzyl 2-(trimethylsilyl)ethyl [(1S,2S,3S)-2-aminocyclopentane-1,3-diyl]biscarbamate

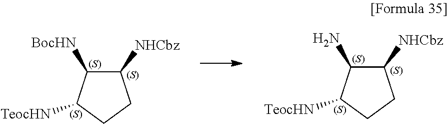

p-Toluenesulfonic acid hydrate (52.2 mg) was added to an ethanol solution (8 mL) of the compound of Reference Example 66 (125 mg) at room temperature, and the resulting mixture was stirred at 80° C. for 7.5 hours. Saturated aqueous sodium bicarbonate was added to the reaction mixture, and the resulting mixture was extracted with ethyl acetate. The organic layer was dried over sodium sulfate, then filtered, and concentrated under reduced pressure to obtain the title compound (101 mg). LC-MS [M+H]⁺/Rt (min): 394.3/0.835 (Method A)

### Reference Example 68

The compound of Reference Example 68 was obtained by using the corresponding raw material compounds according to the method described in Reference Example 58.

TABLE 22

| Reference | | |
|---|---|---|
| Example | Chemical Structure | Physical property data |
| 68 | 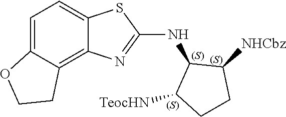 | LC-MS [M + H]⁺/Rt (min): 569.4/1.163 (Method A) |

### Reference Example 69

### tert-Butyl ethyl [(1R,2R,3R,4S)-3-azido-4-methylcyclopentane-1,2-diyl]biscarbamate

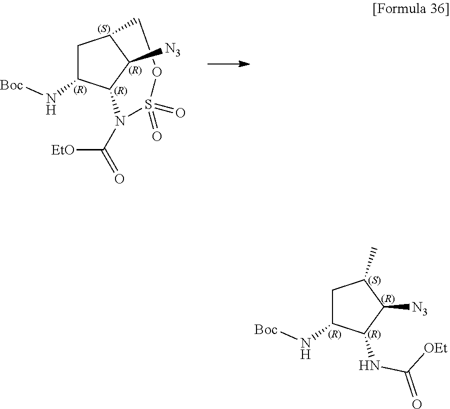

Sodium borohydride (51.4 mg) was added at room temperature to a dimethyl sulfoxide solution (5 mL) of a raw material compound (185 mg) synthesized according to a reference (Angewandte Chemie International Edition 3802 (2009)), and the resulting mixture was stirred at room temperature for 100 minutes. Water and saturated brine were added to the reaction mixture, and the resulting mixture was extracted with ethyl acetate. The organic layer was washed with saturated brine, and then dried over sodium sulfate, filtered, and concentrated under reduced pressure. The residue was purified by silica gel column chromatography (hexane/ethyl acetate) to obtain the title compound (135 mg).

LC-MS [M+Na]⁺/Rt (min): 350.1/1.293 (Method D)

### Reference Example 70

### tert-Butyl ethyl [(1R,2R,3R,4S)-3-azido-4-(fluoromethyl)cyclopentane-1,2-diyl]biscarbamate

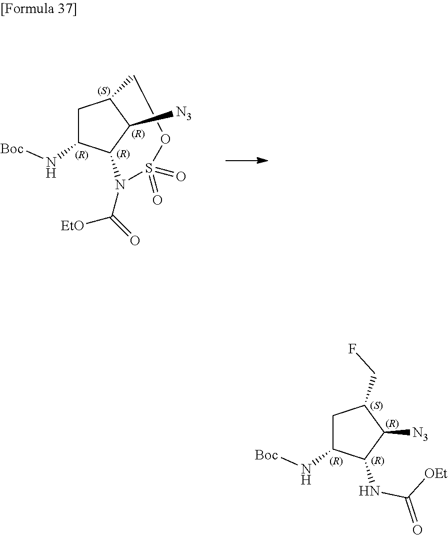

A 1.0 M tetrabutylammonium fluoride tetrahydrofuran solution (1.0 mL) was added at room temperature to a tetrahydrofuran solution (7 mL) of a raw material compound (269 mg) synthesized according to a reference (Angewandte Chemie International Edition 3802 (2009)), and the resulting mixture was stirred at room temperature for 4 hours. A saturated ammonium chloride aqueous solution was added to the reaction mixture, and the resulting mixture was extracted with ethyl acetate. The organic layer was dried over sodium sulfate, filtered, and concentrated under reduced pressure. The residue was purified by silica gel column chromatography (hexane/ethyl acetate) to obtain the title compound (91.6 mg).

LC-MS [M+Na]⁺/Rt (min): 368.2/1.245 (Method D)

### Reference Examples 71 and 72

The compounds of Reference Examples 71 and 72 were obtained by using the corresponding raw material compounds according to the method described in Example 14.

TABLE 23

| Reference | | |
|---|---|---|
| Example | Chemical Structure | Physical property data |
| 71 | 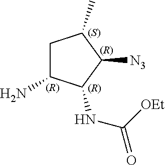 | LC-MS [M + H]⁺/Rt (min): 228.1/0.826 (Method D) |
| 72 | 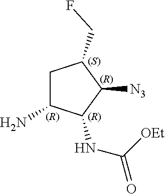 | LC-MS [M + H]⁺/Rt (min): 246.2/0.776 (Method D) |

### Reference Example 73

The compound of Reference Example 73 was obtained by using the corresponding raw material compounds according to the method described in Reference Example 57.

TABLE 24

| Reference | | |
|---|---|---|
| Example | Chemical Structure | Physical property data |
| 73 | 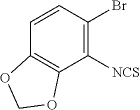 | ¹H-NMR (DMSO-D₆) δ: 7.18 (1H, d, J = 8 .4 Hz), 6.91 (1H, d, J = 8.4 Hz), 6.22 (2H, s). |

### Reference Examples 74 to 77

The compounds of Reference Examples 74 to 77 were obtained by using the corresponding raw material compounds according to the method described in Reference Example 58.

TABLE 25

| Reference | | |
|---|---|---|
| Example | Chemical Structure | Physical property data |
| 74 | 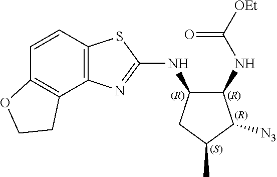 | LC-MS [M + H]⁺/Rt (min): 403.2/1.251 (Method D) |
| 75 | 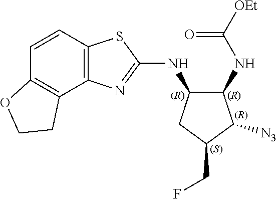 | LC-MS [M + H]⁺/Rt (min): 421.3/1.228 (Method D) |
| 76 | 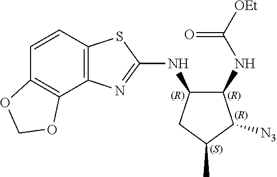 | LC-MS [M + H]⁺/Rt (min): 405.2/1.238 (Method D) |
| 77 | 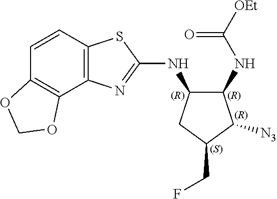 | LC-MS [M + H]⁺/Rt (min): 423.2/1.229 (Method D) |

### Reference Examples 78 to 81

The compounds of Reference Examples 78 to 81 were obtained by using the corresponding raw material compounds according to the method described in Example 51.

TABLE 26

| Reference | | |
|---|---|---|
| Example | Chemical Structure | Physical property data |
| 78 | 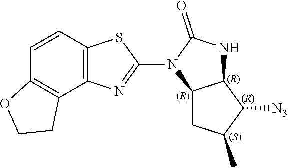 | LC-MS [M + H]⁺/Rt (min): 357.1/1.345 (Method D) |
| 79 | 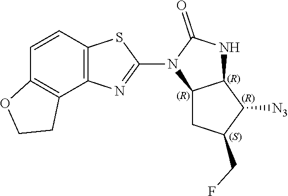 | LC-MS [M + H]⁺/Rt (min): 375.1/1.265 (Method D) |
| 80 | 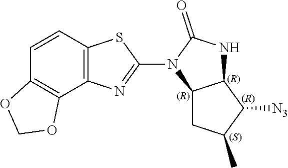 | LC-MS [M + H]⁺/Rt (min): 359.1/1.267 (Method D) |
| 81 | 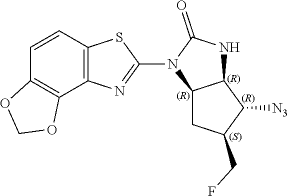 | LC-MS [M + H]⁺/Rt (min): 377.1/1.233 (Method D) |

### Example 1

### tert-Butyl rac-[(3aR,4S,6aS)-1-(7,8-dihydrofuro[3,2-e][1,3]benzothiazol 2-yl)-2-oxooctahydrocyclopenta[d]imidazol 4-yl]methylcarbamate

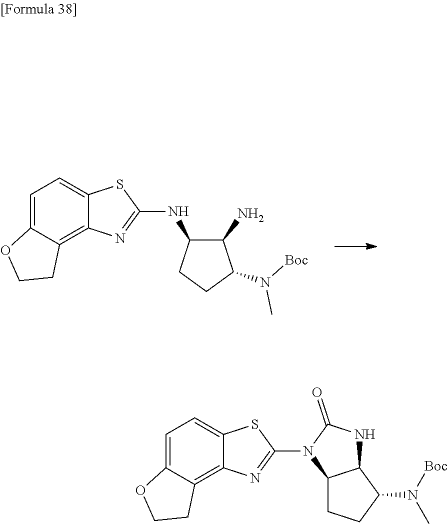 [SMILES: CN(C(=O)OC(C)(C)C)[C@@H]1CC[C@@H](Nc2nc3c4c(ccc3s2)OCC4)[C@H]1N ; CN(C(=O)OC(C)(C)C)[C@@H]1CC[C@@H]2[C@H]1NC(=O)N2c1nc2c3c(ccc2s1)OCC3]

Triethylamine (0.079 mL) and di(N-succinimidyl) carbonate (40 mg) were added to a chloroform solution (7 mL) of the compound of Reference Example 34 (63 mg), and the resulting mixture was stirred for 1 hour. Saturated aqueous sodium bicarbonate and water were added to the reaction mixture, and the resulting mixture was extracted with chloroform. The organic layer was dried over sodium sulfate, filtered, and then concentrated under reduced pressure. The residue was purified by silica gel column chromatography (chloroform/methanol) and reverse phase column chromatography (0.035% trifluoroacetic acid in acetonitrile/0.05% trifluoroacetic acid in water). Methanol was added to the resulting solid, the resulting mixture was stirred, and the solid was collected by filtration and then dried to obtain the title compound (41 mg).

LC-MS [M+H]⁺/Rt (min): 431.3/1.040 (Method A); ¹H-NMR (DMSO-D₆) δ: 8.28 (1H, s), 7.57 (1H, d, J=8.6 Hz), 6.72 (1H, d, J=8.6 Hz), 4.90-4.83 (1H, m), 4.65-4.55 (2H, m), 4.18-4.10 (2H, m), 3.40-3.32 (2H, m), 2.77 (3H, s), 2.59-2.50 (1H, m), 1.83-1.67 (3H, m), 1.42 (9H, s).

### Examples 2 to 7

The compounds of Examples 2 to 7 were obtained by using the corresponding raw material compounds according to the method described in Example 1.

TABLE 27-1

| Example | Chemical Structure | Physical property data |
|---|---|---|
| 2 | 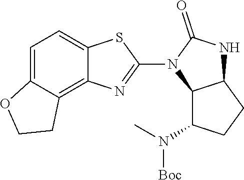 [SMILES: CN(C(=O)OC(C)(C)C)[C@H]1CC[C@@H]2NC(=O)N(c3nc4c5c(ccc4s3)OCC5)[C@@H]21] | LC-MS [M + H]⁺/Rt (min): 431.4/0.964 (Method A); ¹H-NMR (DMSO-D₆) δ: 8.08 (1H, s), 7.52 (1H, d, J = 8.5 Hz), 6.68 (1H, d, J = 8.5 Hz), 4.95-4.88 (1H, m), 4.67-4.52 (2H, m), 4.45-4.36 (1H, m), 4.23-4.15 (1H, m), 3.53-3.41 (1H, m), 3.37- 3.26 (1H, m), 2.91 (3H, s), 2.22- 2.16 (1H, m), 1.88-1.69 (2H, m), 1.58-1.49 (1H, m), 1.04 (9H, br s). |
| 3 | 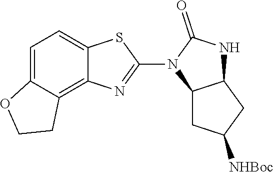 [SMILES: CC(C)(C)OC(=O)N[C@@H]1C[C@@H]2NC(=O)N(c3nc4c5c(ccc4s3)OCC5)[C@@H]2C1] | LC-MS [M + H]⁺/Rt (min): 417.2/0.819 (Method B) |
| 4 | 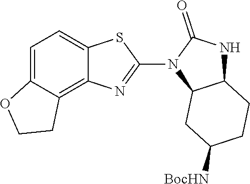 [SMILES: CC(C)(C)OC(=O)N[C@@H]1CC[C@@H]2NC(=O)N(c3nc4c5c(ccc4s3)OCC5)[C@@H]2C1] | LC-MS [M + H]⁺/Rt (min): 331.1/0.451 (Method B |
| 5 | 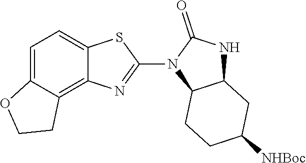 [SMILES: CC(C)(C)OC(=O)N[C@H]1CC[C@@H]2[C@H](C1)NC(=O)N2c1nc2c3c(ccc2s1)OCC3] | LC-MS [M + H]⁺/Rt (min): 331.1/0.491 (Method B). |
| 6 | 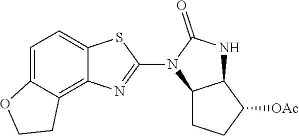 [SMILES: CC(=O)O[C@@H]1CC[C@@H]2[C@H]1NC(=O)N2c1nc2c3c(ccc2s1)OCC3] | LC-MS [M + H]⁺/Rt (min): 360.1/0.782 (Method B); ¹H-NMR (DMSO-D₆) δ: 8.24 (1H, s), 7.58 (1H, d, J = 8.5 Hz), 6.73 (1H, d, J = 8.5 Hz), 5.02-4.97 (1H, m), 4.92-4.89 (1H, m), 4.60 (2H, t, J = 9.1 Hz), 4.13- 4.09 (1H, m), 3.36 (2H, t, J = 9.1 Hz), 2.26-2.07 (2H, m), 2.04 (3H, s), 1.92-1.74 (2H, m). |

TABLE 27-2

| | | |
|---|---|---|
| 7 | 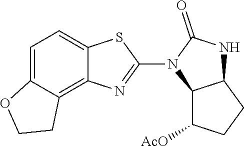 [SMILES: CC(=O)O[C@H]1CC[C@@H]2NC(=O)N(c3nc4c5c(ccc4s3)OCC5)[C@@H]21] | LC-MS [M + H]⁺/Rt (min): 360.1/0.732 (Method B); ¹H-NMR (DMSO-D₆) δ: 8.05 (1H, s), 7.58 (1H, d, J = 8.2 Hz), 6.73 (1H, d, J = 8.2 Hz), 5.44-5.40 (1H, m), 4.81-4.77 (1H, m), 4.60 (2H, t, J = 8.9 Hz), 4.42- 4.37 (1H, m), 3.39-3.21 (2H, m), 2.08 (3H, s), 2.01-1.81 (2H, m), 1.78-1.69 (2H, m). |

### Example 8

### rac-(3aR,6R,6aR)-1-(7,8-Dihydrofuro[3,2-e][1,3]benzothiazol 2-yl)-6-hydroxyhexahydrocyclopenta[d]imidazol 2(1H)-one

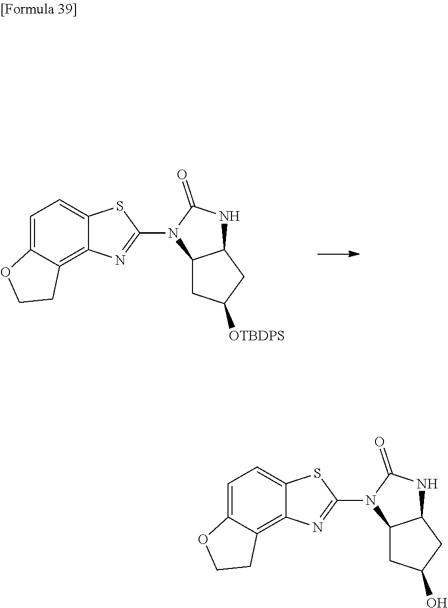

A 1.0 M tetrabutylammonium fluoride tetrahydrofuran solution (1.5 mL) was added to a tetrahydrofuran solution (5 mL) of the compound of Reference Example 41 (555 mg), and the resulting mixture was stirred at room temperature for 0.5 hours and then stirred at 50° C. for 2 hours. The reaction mixture was concentrated under reduced pressure, and the residue was purified by silica gel column chromatography (hexane/ethyl acetate) to obtain the title compound (240 mg).

LC-MS [M+H]⁺/Rt (min): 318.1/0.556 (Method B); ¹H-NMR (DMSO-D₆) δ: 7.92 (1H, brs), 7.54 (1H, d, J=8.8 Hz), 6.69 (1H, d, J=8.8 Hz), 4.90-4.86 (1H, m), 4.60 (2H, t, J=9.2 Hz), 4.23-4.18 (2H, m), 3.39-3.34 (2H, m), 2.30-2.26 (1H, m), 2.07-2.01 (1H, m), 1.92-1.75 (2H, m).

### Example 9

The compound of Example 9 was obtained by using the corresponding raw material compounds according to the method described in Example 8.

TABLE 28

| Example | Chemical Structure | Physical property data |
|---|---|---|
| 9 | 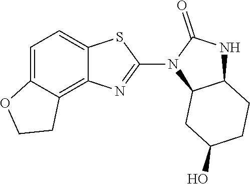 [SMILES: O=C1N[C@H]2CC[C@@H](O)C[C@H]2N1c1nc2c3c(ccc2s1)OCC3] | LC-MS [M + H]⁺/Rt (min): 332.0/0.580 (Method B) |

### Example 10

### rac-(3aR,6R,6aR)-1-(7,8-Dihydrofuro[3,2-e][1,3]benzothiazol 2-yl)-6-hydroxyhexahydrocyclopenta[d]imidazol 2 (1H)-one

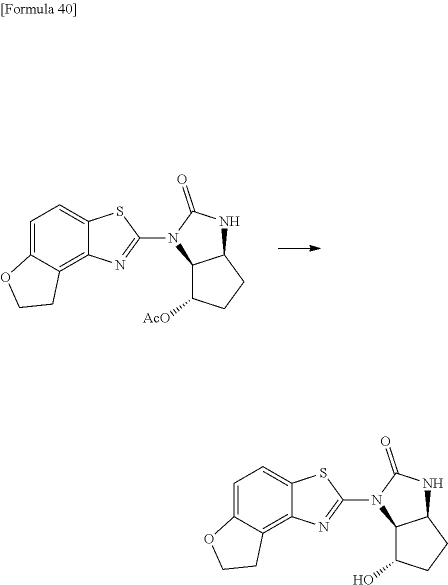 [SMILES: CC(=O)O[C@H]1CC[C@@H]2NC(=O)N(c3nc4c5c(ccc4s3)OCC5)[C@@H]21 ; O=C1N[C@H]2CC[C@H](O)[C@H]2N1c1nc2c3c(ccc2s1)OCC3]

Potassium carbonate (44 mg) was added to a methanol/tetrahydrofuran solution (2 mL/3 mL) of the compound of Example 7 (64 mg), and the resulting mixture was stirred at room temperature for 0.5 hours and then allowed to stand overnight. Saturated aqueous sodium bicarbonate was added to the reaction mixture, and the resulting mixture was extracted with chloroform/ethanol (3/1). The organic layer was dried over sodium sulfate, filtered, and then concentrated under reduced pressure to obtain the title compound (60 mg).

LC-MS [M+H]⁺/Rt (min): 318.1/0.610 (Method B)

¹H-NMR (DMSO-D₆) δ: 7.96 (1H, s), 7.57 (1H, d, J=8.2 Hz), 6.73 (1H, d, J=8.2 Hz), 5.01 (1H, d, J=3.0 Hz), 4.64-4.57 (3H, m), 4.38-4.26 (2H, m), 3.41-3.34 (2H, m), 2.11-1.98 (1H, m), 1.68-1.59 (3H, m).

### Example 11

The compound of Example 11 was obtained by using the corresponding raw material compounds according to the method described in Example 10.

TABLE 29

| Example | Chemical Structure | Physical property data |
|---|---|---|
| 11 | 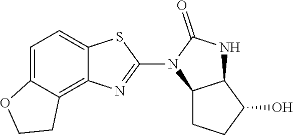 [SMILES: O=C1N[C@H]2[C@H](O)CC[C@H]2N1c1nc2c3c(ccc2s1)OCC3] | LC-MS [M + H]⁺/Rt (min): 318.1/0.612 (Method B); ¹H-NMR (DMSO-D₆) δ: 8.07 (1H, s), 7.56 (1H, d, J = 8.5 Hz), 6.72 (1H, d, J = 8.5 Hz), 5.03-5.00 (1H, m), 4.97-4.91 (1H, m), 4.60 (2H, t, J = 9.1 Hz), 3.97-3.89 (2H, m), 3.43-3.30 (2H, m), 2.21-2.05 (2H, m), 1.68-1.58 (2H, m). |

### Example 12

### rac-(3aR,4R,6aS)-1-(7,8-Dihydrofuro[3,2-e][1,3]benzothiazol 2-yl)-4-(dimethylamino)hexahydrocyclopenta[d]imidazol 2(1H)-one

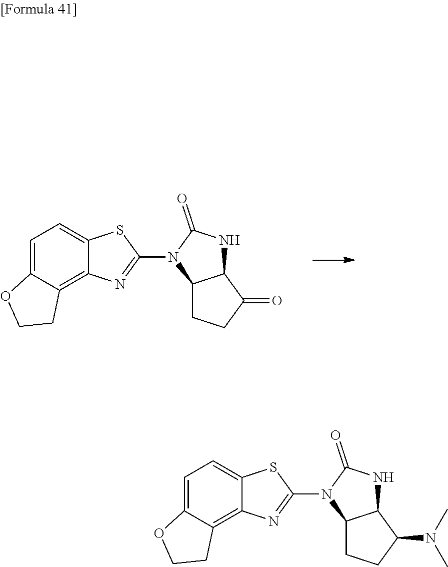 [SMILES: O=C1CC[C@@H]2[C@H]1NC(=O)N2c1nc2c3c(ccc2s1)OCC3 ; CN(C)[C@H]1CC[C@@H]2[C@H]1NC(=O)N2c1nc2c3c(ccc2s1)OCC3]

A 2 M dimethylamine tetrahydrofuran solution (0.032 mL) was added to a tetrahydrofuran solution (1 mL) of the compound of Reference Example 45 (5 mg), the resulting mixture was stirred for 2 hours, then sodium triacetoxyborohydride (17 mg) and tetrahydrofuran (1 mL) were added, and the resulting mixture was stirred overnight. Saturated aqueous sodium bicarbonate was added to the reaction mixture, and the resulting mixture was extracted with chloroform/ethanol (3/1). The organic layer was dried over sodium sulfate, filtered, and then concentrated under reduced pressure. The residue was purified by silica gel column chromatography (chloroform/methanol). Methanol was added to the resulting solid, the resulting mixture was stirred, and the solid was collected by filtration and then dried to obtain the title compound (3 mg).

LC-MS [M+H]⁺/Rt (min): 345.1/0.466 (Method B).

¹H-NMR (DMSO-D₆) δ: 7.78 (1H, s), 7.56 (1H, d, J=8.3 Hz), 6.71 (1H, d, J=8.3 Hz), 4.95-4.89 (1H, m), 4.59 (2H, t, J=8.9 Hz), 4.26-4.21 (1H, m), 3.35 (2H, t, J=8.9 Hz), 2.28-2.22 (1H, m), 2.20 (6H, s), 2.11-2.04 (18, m), 1.94-1.84 (1H, m), 1.79-1.73 (1H, m), 1.48-1.35 (1H, m).

### Example 13

### rac-(3aR,6S,6aR)-1-(7,8-Dihydrofuro[3,2-e](1,3)benzothiazol 2-yl)-6-(dimethylamino) hexahydrocyclopenta[d]imidazol 2 (1H)-one

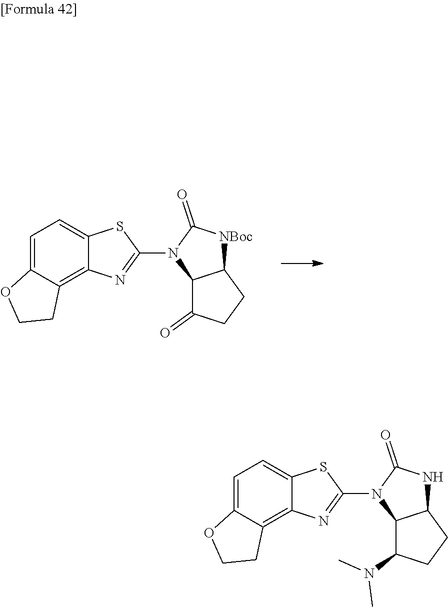 [SMILES: CC(C)(C)OC(=O)N1C(=O)N(c2nc3c4c(ccc3s2)OCC4)[C@@H]2C(=O)CC[C@@H]21 ; CN(C)[C@@H]1CC[C@@H]2NC(=O)N(c3nc4c5c(ccc4s3)OCC5)[C@@H]21]

A 2 M dimethylamine tetrahydrofuran solution (0.240 mL) and acetic acid (one drop) were added to a chloroform solution (2 mL) of the compound of Reference Example 44 (50 mg), the resulting mixture was stirred for 1 hour, then sodium triacetoxyborohydride (127 mg) was added, and the resulting mixture was stirred overnight. Saturated aqueous sodium bicarbonate was added to the reaction mixture, and the resulting mixture was extracted with chloroform/ethanol (3/1). The organic layer was dried over sodium sulfate, filtered, and then concentrated under reduced pressure. The residue was purified by silica gel column chromatography (chloroform/methanol). Trifluoroacetic acid (0.5 mL) was added to a chloroform solution (2 mL) of the resulting solid (30 mg), and the resulting mixture was stirred for 1 hour. Saturated aqueous sodium bicarbonate was added to the reaction mixture, and the resulting mixture was extracted with chloroform/ethanol (3/1). The organic layer was dried over sodium sulfate, filtered, and then concentrated under reduced pressure to obtain the title compound (12 mg).

LC-MS [M+H]⁺/Rt (min): 345.1/0.449 (Method B).

¹H-NMR (DMSO-De) δ: 7.83 (1H, s), 7.53 (1H, d, J=7.9 Hz), 6.70 (1H, d, J=7.9 Hz), 5.05-5.00 (1H, m), 4.59 (2H, t, J=8.9 Hz), 4.27-4.22 (1H, m), 3.46-3.24 (2H, m), 2.72-2.64 (1H, m), 2.22 (6H, s), 1.74-1.58 (4H, m).

### Example 14

### rac-(3aR,4S,6aS)-1-(7,8-Dihydrofuro[3,2-e][1,3]benzothiazol 2-yl)-4-(methylamino)hexahydrocyclopenta[d]imidazol 2(1H)-one

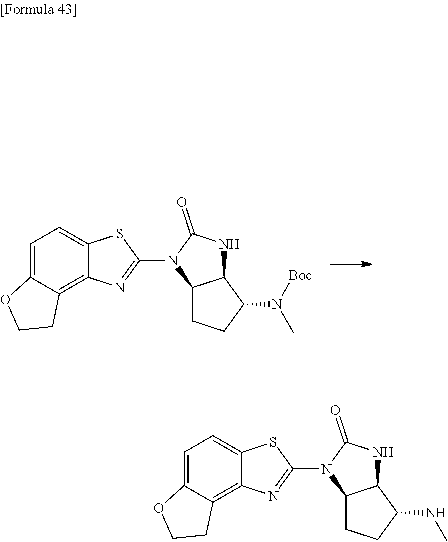 [SMILES: CN(C(=O)OC(C)(C)C)[C@@H]1CC[C@@H]2[C@H]1NC(=O)N2c1nc2c3c(ccc2s1)OCC3 ; CN[C@@H]1CC[C@@H]2[C@H]1NC(=O)N2c1nc2c3c(ccc2s1)OCC3]

Trifluoroacetic acid (4 mL) was added to the compound of Example 1 (40 mg), and the resulting mixture was stirred for 15 minutes. The reaction mixture was concentrated under reduced pressure, saturated aqueous sodium bicarbonate was added to the residue, and the resulting mixture was extracted with chloroform/ethanol (3/1). The organic layer was dried over sodium sulfate, filtered, and then concentrated under reduced pressure. The residue was purified by amino silica gel column chromatography (chloroform/methanol) to obtain the title compound (32 mg).

LC-MS [M+H]⁺/Rt (min): 331.2/0.580 (Method A); ¹H-NMR (DMSO-D₆) δ: 8.04 (1H, s), 7.56 (1H, d, J=8.6 Hz), 6.71 (1H, d, J=8.6 Hz), 4.89-4.85 (1H, m), 4.59 (2H, t, J=8.9 Hz), 3.87 (1H, d, J=7.9 Hz), 3.36 (2H, t, J=8.9 Hz), 2.84-2.81 (1H, m), 2.27 (3H, s), 2.25-2.16 (1H, m), 2.02-1.84 (2H, m), 1.70-1.55 (2H, m).

### Examples 15 and 16

The compounds of Examples 15 and 16 were obtained by using the corresponding raw material compounds according to the method described in Example 14.

TABLE 30

| Example | Chemical Structure | Physical property data |
|---|---|---|
| 15 | 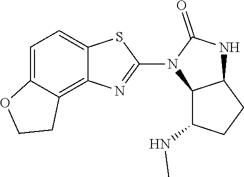 [SMILES: CN[C@H]1CC[C@@H]2NC(=O)N(c3nc4c5c(ccc4s3)OCC5)[C@H]12] | LC-MS [M + H]⁺/Rt (min): 331.2/0.551 (Method A): ¹H-NMR (DMSO-D₆) δ: 7.92 (1H, s), 7.56 (1H, d, J = 8.5 Hz), 6.71 (1H, d, J = 8.5 Hz), 4.62-4.57 (3H, m), 4.30-4.25 (1H, m), 3.39-3.26 (2H, m), 3.18-3.14 (1H, m), 2.51 (3H, s), 2.22-1.97 (2H, m), 1.79- 1.70 (1H, m), 1.65-1.49 (2H, m). |
| 16 | 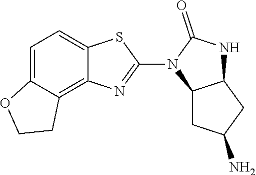 [SMILES: N[C@@H]1C[C@@H]2NC(=O)N(c3nc4c5c(ccc4s3)OCC5)[C@@H]2C1] | LC-MS [M + H]⁺/Rt (min): 317.2/0.443 (Method B) |

### Example 17

### rac-(3aR,5R,6aS)-5-Amino-1-(7,8-dihydrofuro[3,2-e][1,3]benzothiazol 2-yl)hexahydrocyclopenta[d]imidazol 2 (1H)-one

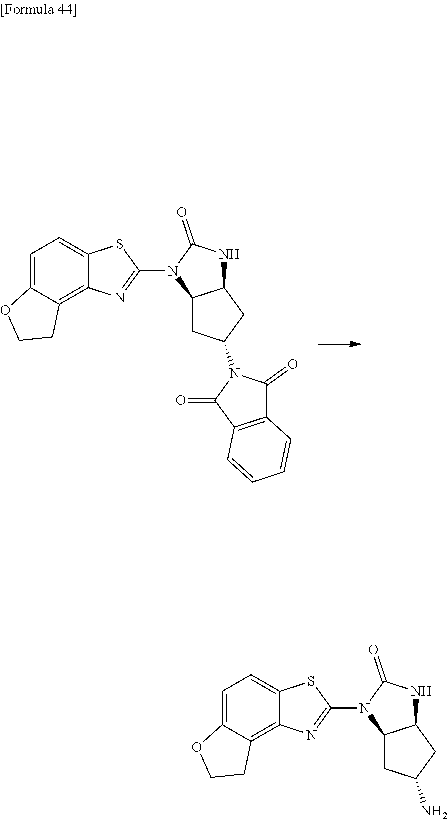 [SMILES: O=C1c2ccccc2C(=O)N1[C@H]1C[C@@H]2NC(=O)N(c3nc4c5c(ccc4s3)OCC5)[C@@H]2C1 ; N[C@H]1C[C@@H]2NC(=O)N(c3nc4c5c(ccc4s3)OCC5)[C@@H]2C1]

Hydrazine monohydrate (50 mg) was added to an ethanol solution (2 mL) of the compound of Reference Example 46 (30 mg), and the resulting mixture was stirred at 80° C. for 2 hours. The reaction mixture was concentrated under reduced pressure, and then the residue was purified by amino silica gel column chromatography (ethyl acetate/methanol) to obtain the title compound (9.5 mg).

LC-MS [M+H]⁺/Rt (min): 317.0/0.448 (Method B); 1H-NMR (CDCl₃) δ: 7.45 (1H, d, J=8.7 Hz), 6.76 (1H, d, J=8.7 Hz), 5.14-5.09 (1H, m), 5.04 (1H, brs), 4.68-4.64 (2H, m), 4.39 (1H, t, J=7.3 Hz), 3.69-3.62 (1H, m), 3.45 (2H, t, J=8.9 Hz), 2.55-2.49 (1H, m), 2.05-2.01 (1H, m), 1.91-1.83 (1H, m), 1.72-1.65 (1H, m).

### Example 18

The compound of Example 18 was obtained by using the corresponding raw material compounds according to the method described in Example 17.

TABLE 31

| | | Physical |
|---|---|---|
| Example | Chemical Structure | property data |
| 18 | 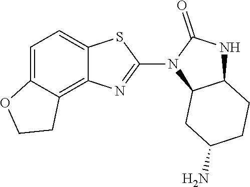 [SMILES: N[C@H]1CC[C@@H]2NC(=O)N(c3nc4c5c(ccc4s3)OCC5)[C@@H]2C1] | LC-MS [M + H]⁺/ Rt (min): 331.0/0.461 (Method B) |

### Example 19

### rac-(3aR,4S,6aS)-1-(7,8-Dihydrofuro[3,2-e][1,3]benzothiazol 2-yl)-4-(dimethylamino) hexahydrocyclopenta[d]imidazol 2 (1H)-one

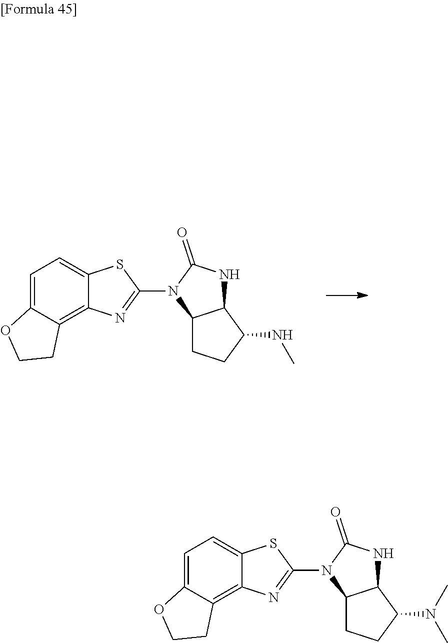 [SMILES: CN[C@@H]1CC[C@@H]2[C@H]1NC(=O)N2c1nc2c3c(ccc2s1)OCC3 ; CN(C)[C@@H]1CC[C@@H]2[C@H]1NC(=O)N2c1nc2c3c(ccc2s1)OCC3]

A 37% formaldehyde solution (0.014 mL) was added to a methanol/tetrahydrofuran (2 mL/1 mL) solution of the compound of Example 15 (15 mg) at room temperature, and the resulting mixture was stirred at room temperature for 1 hour. Sodium triacetoxyborohydride (58 mg) was added at room temperature, and the resulting mixture was stirred at room temperature for 20 minutes. Saturated aqueous sodium bicarbonate was added to the reaction mixture, and the resulting mixture was extracted with chloroform/ethanol (3/1). The organic layer was dried over sodium sulfate, filtered, and then concentrated under reduced pressure. The residue was purified by silica gel column chromatography (chloroform/methanol) to obtain the title compound (13 mg).

LC-MS [M+H]⁺/Rt (min): 345.2/0.582 (Method A); ¹H-NMR (DMSO-D₆) δ: 8.25 (1H, s), 7.56 (1H, d, J=8.2 Hz), 6.72 (1H, d, J=8.2 Hz), 4.86-4.79 (1H, m), 4.62-4.55 (2H, m), 3.97-3.91 (1H, m), 3.40-3.33 (2H, m), 2.46-2.32 (2H, m), 2.19 (6H, s), 1.87-1.78 (1H, m), 1.75-1.65 (1H, m), 1.61-1.51 (1H, m).

### Example 20

### rac-{[(3aR,4S,6aS)-1-(7,8-Dihydrofuro[3,2-e][1,3]benzothiazol 2-yl)-2-oxohexahydrocyclopenta[d]imidazol 4-yl](methyl)amino}acetonitrile

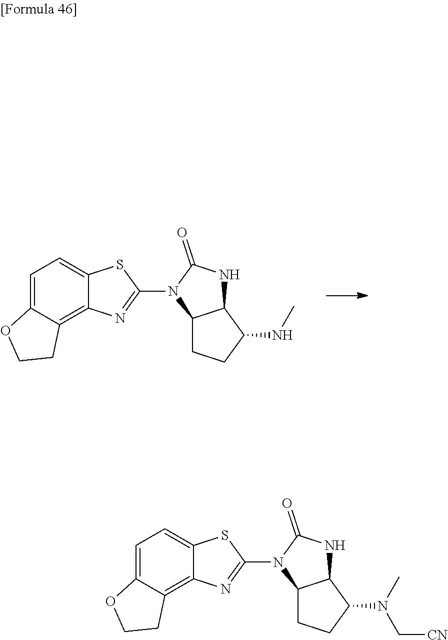 [SMILES: CN[C@@H]1CC[C@@H]2[C@H]1NC(=O)N2c1nc2c3c(ccc2s1)OCC3 ; CN(CC#N)[C@@H]1CC[C@@H]2[C@H]1NC(=O)N2c1nc2c3c(ccc2s1)OCC3]

Acetonitrile bromide (0.002 mL), diisopropylethylamine (0.0057 mL), and potassium carbonate (4.6 mg) were added to a solution of the compound of Example 15 (10 mg) in dimethylformamide (2 mL), the resulting mixture was stirred at room temperature for 4 hours, then bromoacetonitrile (0.001 mL) was added, and the resulting mixture was stirred for 1 hour. Saturated aqueous sodium bicarbonate was added to the reaction mixture, and the resulting mixture was extracted with chloroform. The organic layer was dried over sodium sulfate, filtered, and then concentrated under reduced pressure. The residue was purified by silica gel column chromatography (chloroform/methanol) and reverse phase column chromatography (0.035% trifluoroacetic acid in acetonitrile/0.05% trifluoroacetic acid in water) to obtain the title compound (8.5 mg).

LC-MS [M+H]⁺/Rt (min): 370.3/0.812 (Method A); ¹H-NMR (DMSO-D₆) δ: 8.32-8.30 (1H, m), 7.57 (1H, d, J=8.2 Hz), 6.72 (1H, d, J=8.2 Hz), 4.89-4.82 (1H, m), 4.65-4.55 (2H, m), 4.00-3.95 (1H, m), 3.87-3.72 (2H, m), 3.36 (2H, t, J=8.8 Hz), 2.67-2.59 (1H, m), 2.47-2.40 (1H, m), 2.34 (3H, s), 1.92-1.72 (2H, m), 1.65-1.55 (1H, m).

### Examples 21 to 33

The compounds of Examples 21 to 33 were obtained by using the corresponding raw material compounds according to the method described in Example 13, Example 19, or Example 20.

TABLE 32-1

| Example | Chemical Structure | Physical property data |
|---|---|---|
| 21 | 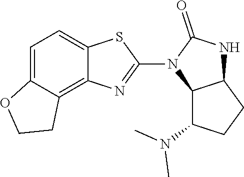 [SMILES: CN(C)[C@H]1CC[C@@H]2NC(=O)N(c3nc4c5c(ccc4s3)OCC5)[C@@H]21] | LC-MS [M + H]⁺/Rt (min): 345.2/0.545 (Method A); ¹H-NMR (DMSO-D₆) δ: 7.94 (1H, s), 7.56 (1H, d, J = 8.2 Hz), 6.71 (1H, d, J = 8.2 Hz), 4.78-4.74 (1H, m), 4.60 (2H, t, J = 8.5 Hz), 4.31-4.25 (1H, m), 3.36-3.27 (2H, m), 2.83-2.78 (1H, m), 2.35 (6H, s), 1.97-1.79 (2H, m), 1.64-1.57 (1H, m), 1.55-1.44 (1H, m). |
| 22 | 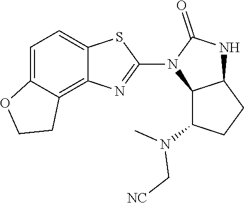 [SMILES: CN(CC#N)[C@H]1CC[C@@H]2NC(=O)N(c3nc4c5c(ccc4s3)OCC5)[C@@H]21] | LC-MS [M + H]⁺/Rt (min): 370.1/0.751 (Method B); ¹H-NMR (DMSO-D₆) δ: 7.99 (1H, s), 7.58 (1H, d, J = 8.5 Hz), 6.73 (1H, d, J = 8.5 Hz), 4.73-4.69 (1H, m), 4.67-4.56 (2H, m), 4.33-4.28 (1H, m), 4.19 (1H, d, J = 17.4 Hz), 3.95 (1H, d, J = 17.4 Hz), 3.50-3.34 (2H, m), 3.05-3.02 (1H, m), 2.44 (3H, s), 1.99- 1.92 (1H, m), 1.86-1.75 (1H, m), 1.69- 1.57 (2H, m). |
| 23 | 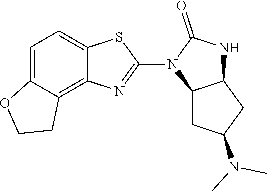 [SMILES: CN(C)[C@@H]1C[C@@H]2NC(=O)N(c3nc4c5c(ccc4s3)OCC5)[C@@H]2C1] | LC-MS [M + H]⁺/Rt (min): 345.1/0.453 (Method B); ¹H-NMR (CDCl₃) δ: 7.45 (1H, d, J = 8.8 Hz), 6.76 (1H, d, J = 8.8 Hz), 5.24 (1H, brs), 4.93-4.87 (1H, m), 4.69-4.63 (2H, m), 4.21-4.15 (1H, m), 3.47 (2H, t, J = 9.4 Hz), 2.80-2.74 (1H, m), 2.63-2.54 (1H, m), 2.40-2.32 (1H, m), 2.23 (6H, s), 1.88-1.80 (1H, m), 1.73-1.66 (1H, m). |
| 24 | 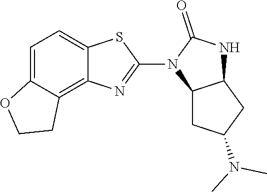 [SMILES: CN(C)[C@H]1C[C@@H]2NC(=O)N(c3nc4c5c(ccc4s3)OCC5)[C@@H]2C1] | LC-MS [M + H]⁺/Rt (min): 345.1/0.453 (Method B); ¹H-NMR (CDCl₃) δ: 7.46 (1H, d, J = 8.8 Hz), 6.76 (1H, d, J = 8.8 Hz), 5.13-5.08 (2H, m), 4.69-4.63 (2H, m), 4.39 (1H, t, J = 7.1 Hz), 3.49-3.44 (2H, m), 2.78-2.70 (1H, m), 2.62-2.56 (1H, m), 2.12-2.06 (1H, m), 1.89-1.81 (1H, m), 1.75-1.67 (1H, m). |

TABLE 32-2

| | | |
|---|---|---|
| 25 | 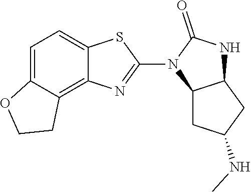 [SMILES: CN[C@H]1C[C@@H]2NC(=O)N(c3nc4c5c(ccc4s3)OCC5)[C@@H]2C1] | LC-MS [M + H]⁺/Rt (min): 331.2/0.454 (Method B) |
| 26 | 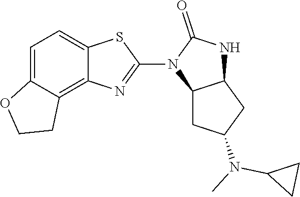 [SMILES: CN(C1CC1)[C@H]1C[C@@H]2NC(=O)N(c3nc4c5c(ccc4s3)OCC5)[C@@H]2C1] | LC-MS [M + H]⁺/Rt (min): 371.1/0.500 (Method B); ¹H-NMR (CDCl₃) δ: 7.45 (1H, d, J = 8.8 Hz), 6.76 (1H, d, J = 8.8 Hz), 5.35-5.33 (1H, m), 5.14-5.04 (1H, m), 4.69-4.64 (2H, m), 4.37 (1H, t, J = 7.2 Hz), 3.49-3.45 (2H, m), 3.16-3.14 (1H, m), 2.65-2.60 (1H, m), 2.36 (s, 3H), 2.18-2.14 (1H, m), 1.95-1.65 (3H, m), 0.53-0.46 (4H, m). |
| 27 | 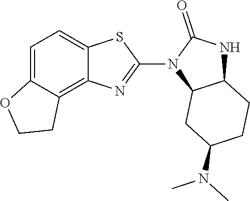 [SMILES: CN(C)[C@@H]1CC[C@@H]2NC(=O)N(c3nc4c5c(ccc4s3)OCC5)[C@@H]2C1] | LC-MS [M + H]⁺/Rt (min): 359.1/0.492 (Method B) |
| 28 | 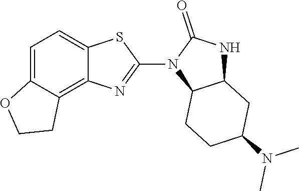 [SMILES: CN(C)[C@H]1CC[C@@H]2[C@H](C1)NC(=O)N2c1nc2c3c(ccc2s1)OCC3] | LC-MS [M + H]⁺/Rt (min): 359.1/0.514 (Method B) |
| 29 | 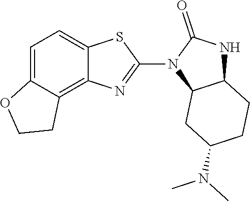 [SMILES: CN(C)[C@H]1CC[C@@H]2NC(=O)N(c3nc4c5c(ccc4s3)OCC5)[C@@H]2C1] | LC-MS [M + H]⁺/Rt (min): 359.1/0.464 (Method B) |
| 30 | 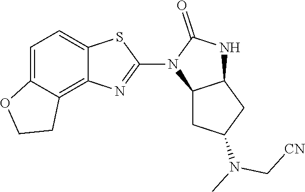 [SMILES: CN(CC#N)[C@H]1C[C@@H]2NC(=O)N(c3nc4c5c(ccc4s3)OCC5)[C@@H]2C1] | LC-MS [M + H]⁺/Rt (min): 370.2/0.652 (Method B) |

TABLE 32-3

| | | |
|---|---|---|
| 31 | 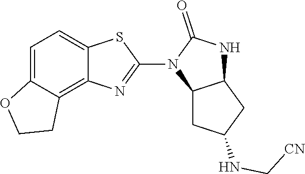 [SMILES: N#CCN[C@H]1C[C@@H]2NC(=O)N(c3nc4c5c(ccc4s3)OCC5)[C@@H]2C1] | LC-MS [M + H]⁺/Rt (min): 356.2/0.553 (Method B) |
| 32 | 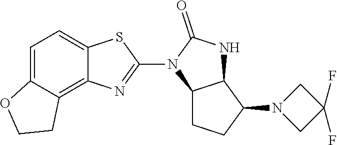 [SMILES: O=C1N[C@@H]2[C@@H](CC[C@@H]2N2CC(F)(F)C2)N1c1nc2c3c(ccc2s1)OCC3] | LC-MS [M + H]⁺/Rt (min): 393.3/0.747 (Method A); ¹H-NMR (DMSO-D₆) δ: 7.88 (1H, s), 7.56 (1H, d, J = 8.3 Hz), 6.71 (1H, d, J = 8.3 Hz), 4.93-4.87 (1H, m), 4.59 (2H, t, J = 9.2 Hz), 4.14-4.09 (1H, m), 3.85-3.74 (2H, m), 3.69-3.59 (2H, m), 3.39-3.32 (2H, m), 2.83-2.76 (1H, m), 2.15-2.08 (1H, m), 1.97-1.86 (1H, m), 1.70-1.62 (1H, m), 1.44-1.31 (1H, m). |
| 33 | 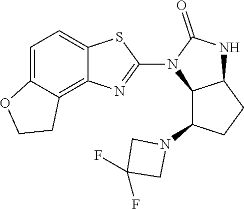 [SMILES: O=C1N[C@H]2CC[C@@H](N3CC(F)(F)C3)[C@H]2N1c1nc2c3c(ccc2s1)OCC3] | LC-MS [M + H]⁺/Rt (min): 393.2/0.696 (Method B); ¹H-NMR (DMSO-D₅) δ: 8.01 (1H, s), 7.57 (1H, d, J = 8.6 Hz), 6.72 (1H, d, J = 8.6 Hz), 4.79 (1H, dd, J = 9.5, 5.2 Hz), 4.61 (2H, t, J = 8.9 Hz), 4.21-4.14 (1H, m), 3.47-3.26 (7H, m), 1.96-1.86 (1H, m), 1.75-1.60 (3H, m). |

### Example 34

### rac-(3aR,6R,6aR)-6-(3,3-Difluoroazetidin-1-yl)-1-(7,8-dihydrofuro[3,2-e][1,3]benzothiazole 2-yl) hexahydrocyclopenta[d]imidazol 2 (1H)-one

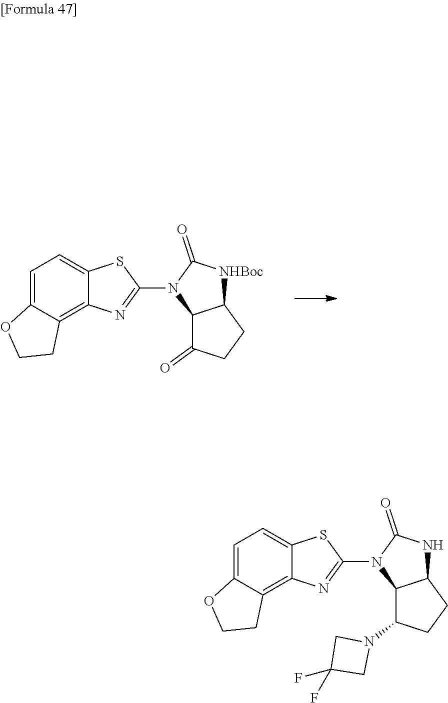

3,3-Difluoroazetidine hydrochloride (52 mg), triethylamine (0.056 mL), and acetic acid (0.057 mL) were added to a methanol/tetrahydrofuran solution (0.5 mL/0.2 mL) of the compound of Reference Example 44 (42 mg) at room temperature, and the resulting mixture was stirred at 60° C. for 2.5 hours. Sodium cyanoborohydride (25 mg) was added at 60° C., and the resulting mixture was stirred at 60° C. for 40 minutes. The reaction mixture was cooled to room temperature, then saturated aqueous sodium bicarbonate was added, and the resulting mixture was extracted with chloroform/ethanol (3/1). The organic layer was dried over sodium sulfate, filtered, and then concentrated under reduced pressure. The residue was purified by silica gel column chromatography (chloroform/methanol). Trifluoroacetic acid (0.2 mL) was added to a chloroform solution (2 mL) of the resulting solid (33 mg), and the resulting mixture was stirred for 3.5 hours. Saturated aqueous sodium bicarbonate was added to the reaction mixture, and the resulting mixture was extracted with chloroform/ethanol (3/1). The organic layer was dried over sodium sulfate, filtered, and then concentrated under reduced pressure. The residue was purified by reverse phase column chromatography (0.035% trifluoroacetic acid in acetonitrile/0.05% trifluoroacetic acid in water) to obtain the title compound (15 mg).

LC-MS [M+H]⁺/Rt (min): 393.2/0.807 (Method B); ¹H-NMR (DMSO-D₆) δ: 7.96 (1H, s), 7.58 (1H, d, J=8.2 Hz), 6.73 (1H, d, J=8.2 Hz), 4.69-4.57 (2H, m), 4.50-4.47 (1H, m), 4.36-4.31 (1H, m), 4.13-4.02 (2H, m), 3.78-3.67 (2H, m), 3.46-3.25 (3H, m), 2.01-1.90 (1H, m), 1.66-1.52 (3H, m).

### Example 35

### rac-(3aR,5S,6aS)-1-(7,8-Dihydrofuro[3,2-e][1,3]benzothiazol 2-yl)-5-methoxyhexahydrocyclopenta[d]imidazol 2(1H)-one

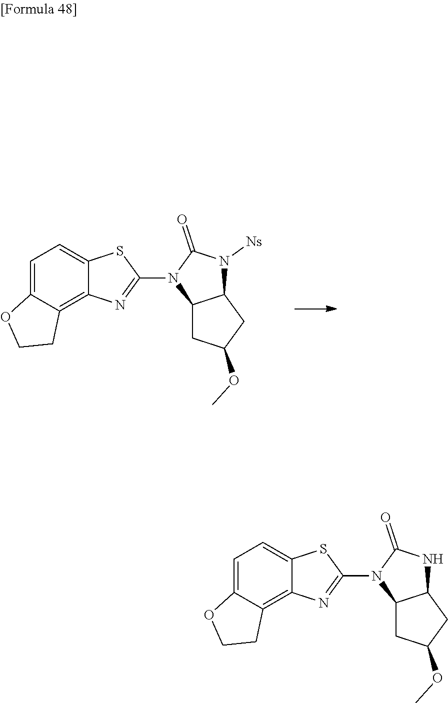

Potassium carbonate (55 mg) and thiophenol (0.020 mL) were added to a dimethylformamide solution (1 mL) of the compound of Reference Example 50 (22 mg), and the resulting mixture was stirred at room temperature for 4 hours. Water was added to the reaction mixture, and the resulting mixture was extracted with ethyl acetate. The organic layer was dried over sodium sulfate, filtered, and then concentrated under reduced pressure. The residue was purified by silica gel column chromatography (ethyl acetate/methanol) to obtain the title compound (5.0 mg).

LC-MS [M+H]⁺/Rt (min): 332.1/0.616 (Method B)

### Example 36

The compound of Example 36 was obtained by using the corresponding raw material compounds according to the method described in Example 1.

TABLE 33

| Example | Chemical Structure | Physical property data |
|---|---|---|
| 36 | 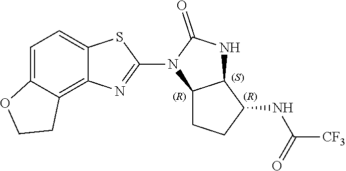 | LC-MS [M + H]⁺/Rt (min): 413.2/0.780 (Method B); ¹H-NMR (DMSO-D₆) δ: 9.55 (1H, s), 8.24 (1H, s), 7.58 (1H, d, J = 8.5 Hz), 6.73 (1H, d, J = 8.5 Hz), 4.98-4.93 (1H, m), 4.60 (2H, t, J = 9.2 Hz), 4.14-4.11 (1H, m), 4.07-4.03 (1H, m), 3.36 (2H, t, J = 9.2 Hz), 2.35-2.25 (1H, m), 2.11-2.03 (1H, m), 1.95-1.86 (1H, m), 1.73-1.66 (1H, m). |

### Example 37

### (3aS,4R,6aR)-4-Amino-1-(7,8-dihydrofuro[3,2-e][1,3]benzothiazol 2-yl)hexahydrocyclopenta[d]imidazol 2 (1H)-one

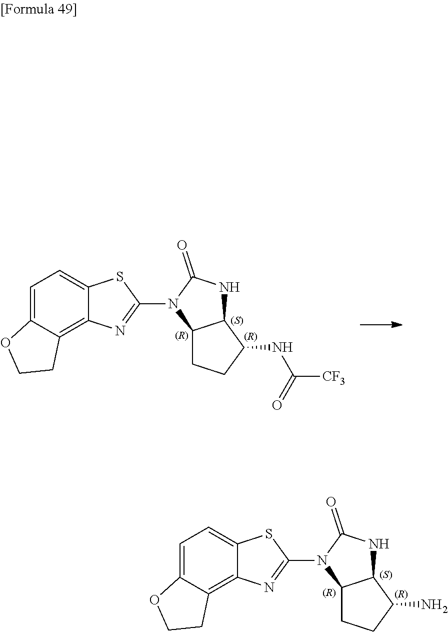

Potassium carbonate (48 mg) was added to a methanol/tetrahydrofuran solution (2 mL/2 mL) of the compound of Example 36 (72 mg), and the resulting mixture was stirred at room temperature for 1 hour. Water (1.5 mL) was added, and the resulting mixture was stirred at room temperature for 1 hour, then potassium carbonate (48 mg) was added, and the resulting mixture was stirred at 70° C. for 2 hours. Saturated aqueous sodium bicarbonate and potassium carbonate were added to the reaction mixture, and the resulting mixture was extracted with chloroform/ethanol (3/1). The organic layer was dried over sodium sulfate, filtered, and then concentrated under reduced pressure. Methanol was added to the residue, the resulting mixture was stirred, and the resulting solid was collected by filtration and then dried to obtain the title compound (43 mg).

LC-MS [M+H]⁺/Rt (min): 317.2/0.489 (Method B); ¹H-NMR (DMSO-D₆) δ: 8.02 (1H, s), 7.56 (1H, d, J=8.2 Hz), 6.71 (1H, d, J=8.2 Hz), 4.94-4.89 (1H, m), 4.60 (2H, t, J=8.8 Hz), 3.80-3.76 (1H, m), 3.36 (2H, t, J=8.8 Hz), 3.22-3.18 (1H, m), 2.37-2.25 (1H, m), 2.00-1.92 (1H, m), 1.75-1.53 (3H, m), 1.49-1.41 (1H, m).

### Examples 38 and 39

The compounds of Examples 38 and 39 were obtained by using the corresponding raw material compounds according to the method described in Example 13 or Example 34.

TABLE 34

| Example | Chemical Structure | Physical property data |
|---|---|---|
| 38 | 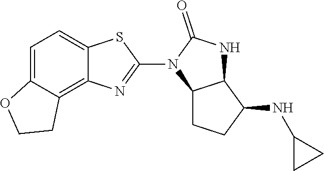 [SMILES: O=C1N[C@H]2[C@@H](NC3CC3)CC[C@H]2N1c1nc2c3c(ccc2s1)OCC3] | LC-MS [M + H]⁺/Rt (min): 357.2/0.527 (Method B); ¹H-NMR (DMSO-D₆) δ: 7.79 (1H, s), 7.56 (1H, d, J = 7.9 Hz), 6.71 (1H, d, J = 7.9 Hz), 4.93-4.88 (1H, m), 4.59 (2H, t, J = 9.2 Hz), 4.22-4.17 (1H, m), 3.35 (2H, t, J = 9.2 Hz), 3.07-3.02 (1H, m), 2.32-2.16 (2H, m), 2.07-2.00 (1H, m), 1.90-1.71 (2H, m), 1.36- 1.25 (1H, m), 0.41-0.19 (4H, m). |
| 39 | 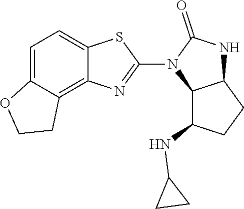 [SMILES: O=C1N[C@H]2CC[C@@H](NC3CC3)[C@H]2N1c1nc2c3c(ccc2s1)OCC3] | LC-MS [M + H]⁺/Rt (min): 357.2/0.459 (Method B); ¹H-NMR (DMSO-D₆) δ: 7.91 (1H, s), 7.55 (1H, d, J = 8.5 Hz), 6.71 (1H, d, J = 8.5 Hz), 4.93-4.88 (1H, m), 4.65-4.56 (2H, m), 4.22-4.16 (1H, m), 3.48-3.23 (3H, m), 2.24-2.11 (2H, m), 1.85- 1.56 (4H, m), 0.35-0.28 (1H, m), 0.23-0.15 (1H, m), 0.08-0.00 (2H, m). |

### Examples 40 to 44

The compounds of Examples 40 to 44 were obtained by using the corresponding raw material compounds according to the method described in Example 19.

TABLE 35-1

| Example | Chemical Structure | Physical property data |
|---|---|---|
| 40 | 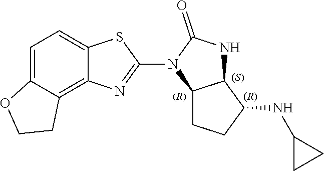 | LC-MS [M + H]⁺/Rt (min): 357.2/0.739 (Method A); ¹H-NMR (DMSO-D₆) δ: 8.05 (1H, s), 7.56 (1H, d, J = 8.3 Hz), 6.71 (1H, d, J = 8.3 Hz), 4.89-4.85 (1H, m), 4.59 (2H, t, J = 9.2 Hz), 3.97-3.92 (1H, m), 3.35 (2H, t, J = 9.2 Hz), 3.10-3.06 (1H, m), 2.41-2.32 (1H, m), 2.27-2.16 (1H, m), 2.07-2.02 (1H, m), 1.95-1.92 (1H, m), 1.71-1.64 (2H, m), 0.42-0.37 (2H, m), 0.25-0.21 (2H, m). |
| 41 | 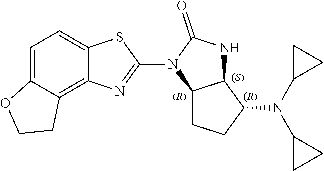 | LC-MS [M + H]⁺/Rt (min): 397.2/0.640 (Method B); ¹H-NMR (DMSO-D₆) δ: 8.12 (1H, s), 7.56 (1H, d, J = 8.6 Hz), 6.72 (1H, d, J = 8.6 Hz), 4.85-4.78 (1H, m), 4.64-4.55 (2H, m), 4.27-4.21 (1H, m), 3.40-3.33 (2H, m), 3.17-3.10 (1H, m), 2.58- 2.51 (1H, m), 2.00-1.95 (2H, m), 1.89-1.80 (2H, m), 1.76-1.65 (1H, m), 0.54-0.34 (8H, m). |
| 42 | 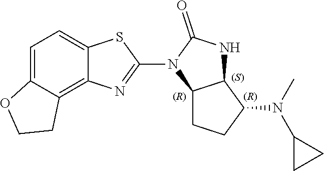 | LC-MS [M + H]⁺/Rt (min): 371.2/0.527 (Method B); ¹H-NMR (DMSO-D₆) δ: 8.18 (1H, s), 7.56 (1H, d, J = 8.6 Hz), 6.72 (1H, d, J = 8.6 Hz), 4.84-4.77 (1H, m), 4.64- 4.55 (2H, m), 4.10-4.05 (1H, m), 3.39-3.33 (2H, m), 2.81-2.75 (1H, m), 2.47-2.41 (1H, m), 2.29 (3H, s), 1.91-1.82 (1H, m), 1.77- 1.65 (3H, m), 0.57-0.46 (2H, m), 0.40-0.32 (2H, m). |

TABLE 35-2

| | | |
|---|---|---|
| 43 | 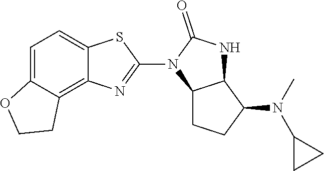 [SMILES: CN(C1CC1)[C@H]1CC[C@@H]2[C@H]1NC(=O)N2c1nc2c3c(ccc2s1)OCC3] | LC-MS [M + H]⁺/Rt (min): 371.2/0.523 (Method B); ¹H-NMR (DMSO-D₆) δ: 7.62 (1H, s), 7.56 (1H, d, J = 8.3 Hz), 6.71 (1H, d, J = 8.3 Hz), 4.93-4.88 (1H, m), 4.59 (2H, t, J = 9.2 Hz), 4.29-4.24 (1H, m), 3.35 (2H, t, J = 9.2 Hz), 2.68-2.61 (1H, m), 2.32 (3H, s), 2.09-2.01 (1H, m), 1.94-1.82 (2H, m), 1.74-1.67 (1H, m), 1.61-1.48 (1H, m), 0.51-0.34 (4H, m). |
| 44 | 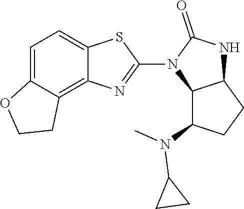 [SMILES: CN(C1CC1)[C@@H]1CC[C@@H]2NC(=O)N(c3nc4c5c(ccc4s3)OCC5)[C@@H]21] | LC-MS [M + H]⁺/Rt (min): 371.2/0.496 (Method B); ¹H-NMR (DMSO-D₆) δ: 7.77 (1H, s), 7.52 (1H, d, J = 8.6 Hz), 6.68 (1H, d, J = 8.6 Hz), 5.02-4.96 (1H, m), 4.66-4.54 (2H, m), 4.24-4.18 (1H, m), 3.46-3.24 (3H, m), 2.18 (3H, s), 2.03-1.87 (2H, m), 1.78-1.60 (3H, m), 0.32- 0.18 (2H, m), 0.16-0.09 (1H, m), −0.07-−0.16 (1H, m). |

### Examples 45 to 48

The compounds of Examples 45 to 48 were obtained by using the corresponding raw material compounds according to the method described in Example 13 or Example 34.

TABLE 36

| Example | Chemical Structure | Physical property data |
|---|---|---|
| 45 | 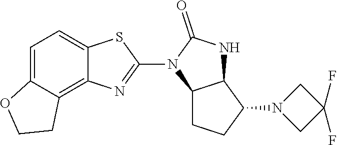 [SMILES: O=C1N[C@H]2[C@H](N3CC(F)(F)C3)CC[C@H]2N1c1nc2c3c(ccc2s1)OCC3] | LC-MS [M + H]⁺/Rt (min): 393.2/0.771 (Method B); ¹H-NMR (DMSO-D₆) δ: 8.00 (1H, s), 7.57 (1H, d, J = 8.2 Hz), 6.72 (1H, d, J = 8.2 Hz), 4.94-4.89 (1H, m), 4.60 (2H, t, J = 9.1 Hz), 3.86-3.82 (1H, m), 3.70-3.52 (4H, m), 3.35 (2H, t, J = 9.1 Hz), 2.86-2.81 (1H, m), 2.21-2.11 (1H, m), 2.09-2.01 (1H, m), 1.70-1.61 (1H, m), 1.56-1.48 (1H, m). |
| 46 | 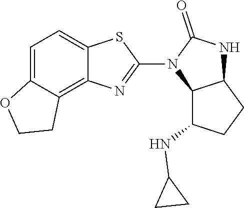 [SMILES: O=C1N[C@H]2CC[C@H](NC3CC3)[C@H]2N1c1nc2c3c(ccc2s1)OCC3] | LC-MS [M + H]⁺/Rt (min): 357.2/0.499 (Method B); ¹H-NMR (DMSO-D₆) δ: 7.96 (1H, s), 7.57 (1H, d, J = 8.3 Hz), 6.72 (1H, d, J = 8.3 Hz), 4.67-4.55 (3H, m), 4.26-4.21 (1H, m), 3.45-3.40 (1H, m), 3.39-3.22 (2H, m), 2.70-2.62 (1H, m), 2.33- 2.28 (1H, m), 2.07-1.98 (1H, m), 1.81-1.72 (1H, m), 1.61-1.52 (2H, m), 0.51-0.41 (2H, m), 0.34-0.21 (2H, m). |
| 47 | 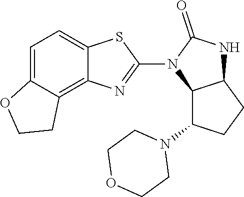 [SMILES: O=C1N[C@H]2CC[C@H](N3CCOCC3)[C@H]2N1c1nc2c3c(ccc2s1)OCC3] | LC-MS [M + H]⁺/Rt (min): 387.2/0.495 (Method B); ¹H-NMR (DMSO-D₆) δ: 7.95 (1H, s), 7.56 (1H, d, J = 8.2 Hz), 6.71 (1H, d, J = 8.2 Hz), 4.78-4.74 (1H, m), 4.66-4.55 (2H, m), 4.34-4.28 (1H, m), 3.64-3.58 (4H, m), 3.35-3.26 (2H, m), 2.94- 2.84 (3H, m), 2.56-2.51 (2H, m), 2.02-1.95 (1H, m), 1.87-1.76 (1H, m), 1.64-1.44 (2H, m). |
| 48 | 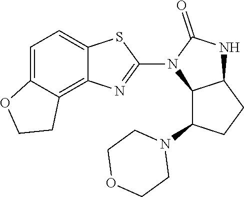 [SMILES: O=C1N[C@H]2CC[C@@H](N3CCOCC3)[C@H]2N1c1nc2c3c(ccc2s1)OCC3] | LC-MS [M + H]⁺/Rt (min): 387.2/0.455 (Method B); ¹H-NMR (DMSO-D₆) δ: 7.81 (1H, s), 7.55 (1H, d, J = 8.3 Hz), 6.70 (1H, d, J = 8.3 Hz), 5.09-5.05 (1H, m), 4.63-4.54 (2H, m), 4.27-4.22 (1H, m), 3.39-3.29 (1H, m), 3.24-3.13 (3H, m), 3.04- 2.85 (4H, m), 2.80-2.74 (1H, m), 2.34-2.30 (2H, m), 1.74-1.52 (4H, m). |

### Examples 49 and 50

The compounds of Examples 49 and 50 were obtained by using the corresponding raw material compounds according to the method described in Example 19.

TABLE 37

| Example | Chemical Structure | Physical property data |
|---|---|---|
| 49 | 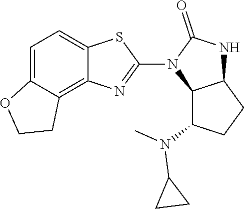 [SMILES: CN(C1CC1)[C@H]1CC[C@@H]2NC(=O)N(c3nc4c5c(ccc4s3)OCC5)[C@@H]21] | LC-MS [M + H]⁺/Rt (min): 371.2/0.513 (Method B); ¹H-NMR (DMSO-D₆) δ: 7.94 (1H, s), 7.56 (1H, d, J = 8.5 Hz) 6.71 (1H, d, J = 8.5 Hz), 4.97-4.93 (1H, m), 4.60 (2H, t, J = 9.1 Hz), 4.32-4.27 (1H, m), 3.39- 3.28 (2H, m), 3.21-3.17 (1H, m), 2.52 (3H, s), 2.25-2.17 (1H, m), 1.96-1.79 (2H, m), 1.67-1.52 (2H, m), 0.56-0.38 (3H, m), 0.36-0.30 (1H, m). |
| 50 | 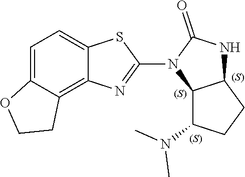 | LC-MS [M + H]⁺/Rt (min): 345.2/0.557 (Method A); ¹H-NMR (DMSO-D₅) δ: 7.94 (1H, s), 7.56 (1H, d, J = 8.5 Hz), 6.71 (1H, d, J = 8.5 Hz), 4.78-4.74 (1H, m), 4.60 (2H, t, J = 8.9 Hz), 4.31-4.25 (1H, m), 3.36- 3.27 (2H, m), 2.83-2.78 (1H, m), 2.35 (6H, s), 1.97-1.79 (2H, m), 1.64-1.57 (1H, m), 1.55-1.44 (1H, m). |

### Example 51

### 2-(Trimethylsilyl)ethyl [(3aS,4S,6aS)-3-(7,8-dihydrofuro[3,2-e][1,3]benzothiazol-2-yl)-2-oxooctahydrocyclopenta[d]imidazol-4-yl]carbamate

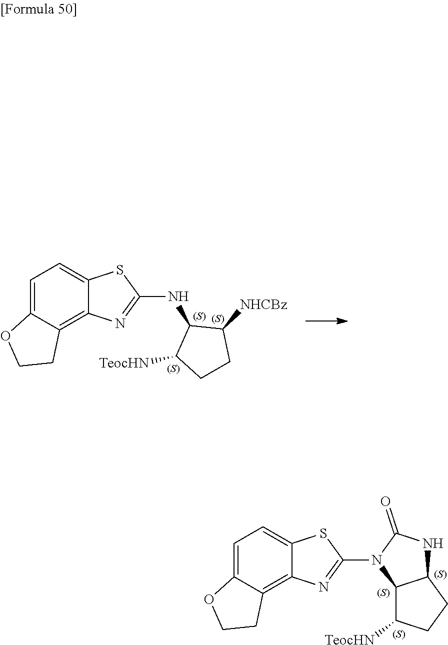

tert-Butoxypotassium (28.1 mg) was added to a tetrahydrofuran solution (10 mL) of the compound of Reference Example 68 (68.2 mg) at room temperature, and the resulting mixture was stirred at room temperature for 2 hours. Saturated aqueous ammonium chloride was added to the reaction mixture, and the resulting mixture was extracted with chloroform. The organic layer was dried over sodium sulfate, then filtered, and concentrated under reduced pressure. The residue was purified by silica gel column chromatography (chloroform/methanol) to obtain the title compound (48.8 mg).

LC-MS [M+H]⁺/Rt (min): 461.4/1.094 (Method A)

### Example 52

### (3aS,6S,6aR)-6-Amino-1-(7,8-dihydrofuro[3,2-e][1,3]benzothiazol-2-yl)hexahydrocyclopenta[d]imidazol-2 (1H)-one

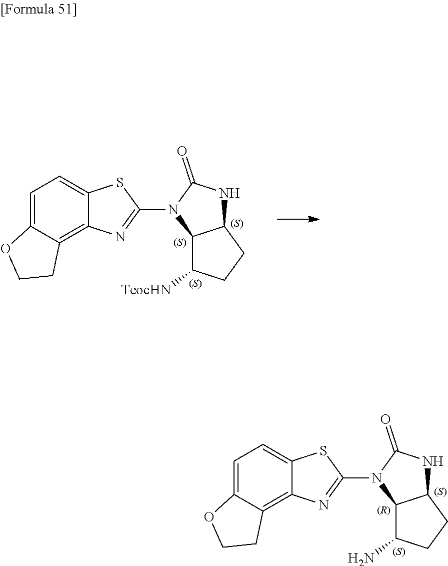

Trifluoroacetic acid (3 mL) was added to a chloroform solution (1 mL) of the compound of Example 51 (48.8 mg) at room temperature, and the resulting mixture was stirred at room temperature for 1.5 hours and then stirred at 60° C. for 2.5 hours. The reaction mixture was concentrated under reduced pressure. Saturated aqueous sodium bicarbonate was added to the residue, and the resulting mixture was extracted with chloroform. The organic layer was dried over sodium sulfate, then filtered, and concentrated under reduced pressure to obtain the title compound (37.9 mg).

LC-MS [M+H]⁺/Rt (min): 317.2/0.541 (Method A)

### Examples 53 to 56

The compounds of Examples 53 to 56 were obtained by using the corresponding raw material compounds according to the method described in Reference Example 54.

TABLE 38

| Example | Chemical Structure | Physical property data |
|---|---|---|
| 53 | 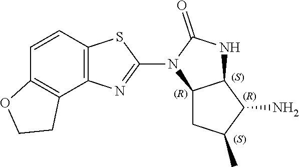 | LC-MS [M + H]⁺/Rt (min): 331.1/0.938 (Method D) |
| 54 | 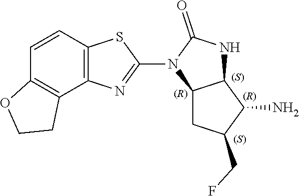 | LC-MS [M + H]⁺/Rt (min): 349.1/0.928 (Method D) |
| 55 | 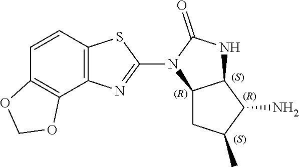 | LC-MS [M + H]⁺/Rt (min): 333.1/0.920 (Method D) |
| 56 | 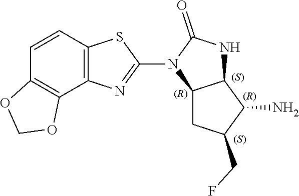 | LC-MS [M + H]⁺/Rt (min): 351.0/0.919 (Method D) |

### Examples 57 to 60

The compounds of Examples 57 to 60 were obtained by using the corresponding raw material compounds according to the method described in Example 19.

TABLE 39

| Example | Chemical Structure | Physical property data |
|---|---|---|
| 57 | 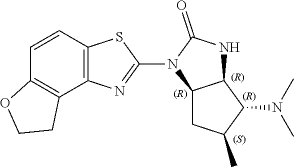 | LC-MS [M + H]⁺/Rt (min): 359.2/0.938 (Method D); ¹H-NMR (DMSO-D₆) δ: 8.25 (1H, s), 7.56 (1H, d, J = 8.5 Hz), 6.71 (1H, d, J = 8.5 Hz), 4.74-4.67 (1H, m), 4.64-4.55 (2H, m), 4.07-4.01 (1H, m), 3.38-3.28 (3H, m), 2.70-2.62 (1H, m), 2.48- 2.42 (1H, m), 2.27 (6H, s), 2.01- 1.92 (1H, m), 1.33-1.24 (1H, m), 0.99 (3H, d, J = 6.1 Hz). |
| 58 | 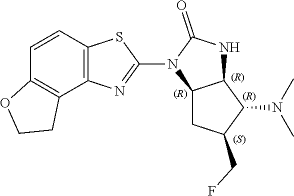 | LC-MS [M + H]⁺/Rt (min): 377.1/0.923 (Method D); ¹H-NMR (DMSO-D₆) δ: 8.31 (1H, s), 7.57 (1H, d, J = 8.5 Hz), 6.72 (1H, d, J = 8.5 Hz), 4.84-4.77 (1H, m), 4.63-4.57 (2H, m), 4.56-4.44 (1H, m), 4.44-4.31 (1H, m), 4.15-4.10 (1H, m), 3.39- 3.32 (2H, m), 2.92-2.77 (1H, m), 2.73-2.63 (1H, m), 2.43-2.29 (1H, m), 2.27 (6H, s), 1.61-1.53 (1H, m). |
| 59 | 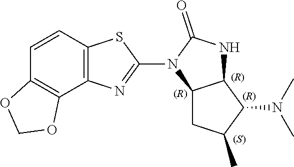 | LC-MS [M + H]⁺/Rt (min): 361.1/0.939 (Method D); ¹H-NMR (DMSO-D₆) δ: 8.30 (1H, s), 7.33 (1H, d, J = 8.3 Hz), 6.90 (1H, d, J = 8.3 Hz), 6.08 (2H, s), 4.73-4.64 (1H, m), 4.08- 3.99 (1H, m), 2.67-2.59 (1H, m), 2.47-2.42 (1H, m), 2.27 (6H, s), 2.01-1.89 (1H, m), 1.38-1.26 (1H, m), 0.99 (3H, d, J = 6.7 Hz). |
| 60 | 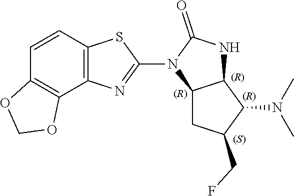 | LC-MS [M + H]⁺/Rt (min): 379.2/0.902 (Method D); ¹H-NMR (DMSO-D₆) δ: 8.36 (1H, s), 7.34 (1H, d, J = 8.2 Hz), 6.91 (1H, d, J = 8.2 Hz), 6.10-6.08 (2H, m), 4.83-4.74 (1H, m), 4.55-4.29 (2H, m), 4.17-4.11 (1H, m), 2.82-2.76 (1H, m), 2.69- 2.62 (1H, m), 2.44-2.30 (1H, m), 2.27 (6H, s), 1.63-1.54 (1H, m). |

### Test Example 1

### [Inhibition Test on Activity of DYRK Family (DYRK1A, DYRK1B, DYRK2, and DYRK3)]

### (Method for Measuring Kinase Activity)

The kinase activity was measured by mobility shift assay (MSA) method using QuickScout Screening Assist™ MSA (commercially available kit manufactured by Carna Biosciences, Inc.). The substrate of the kinase reaction used was an FITC-labeled DYRKtide peptide included in the kit. An assay buffer [20 mM HEPES, 0.01% Triton X-100™, 2 mM dithiothreitol, pH 7.5] was used to create a substrate mixture solution with a substrate (4 μM), MgCl₂ (20 mM), and ATP (DYRK1A: 100 μM; DYRK1B: 200 μM; DYRK2: 40 μM; and DYRK3: 20 μM). In addition, kinases (DYRK1A: manufactured by Carna Biosciences, Inc., Cat. No. 04-130; DYRK1B: manufactured by Carna Biosciences, Inc., Cat. No. 04-131; DYRK2; manufactured by Carna Biosciences, Inc., Cat. No. 04-132; and DYRK3; manufactured by Carna Biosciences, Inc., Cat. No. 04-133) were diluted with the assay buffer to prepare enzyme solutions (DYRK1A: 0.2 ng/μL; DYRK1B: 0.08 ng/μL; DYRK2: 0.04 ng/μL; and DYRK3: 0.25 ng/μL). The 10 mM solution of the test compound in DMSO was further diluted with DMSO to 10 levels of the concentration (0.00003 mM, 0.0001 mM, 0.0003 mM, 0.001 mM, 0.003 mM, 0.01 mM, 0.03 mM, 0.1 mM, 0.3 mM, and 1 mM), each of which was subjected to 25-fold dilution with the assay buffer to obtain a drug solution (4% DMSO solution). 5 μL of the drug solution or a control solution (4% DMSO-assay buffer), 5 μL of the substrate mixture solution, and 10 μL of the enzyme solution were mixed in the wells of a polypropylene 384-well plate and allowed to react at room temperature for 1 hour, and then the reaction was quenched by adding 60 μL of the termination buffer included in the kit. Subsequently, the quantities of the substrates (S) and the phosphorylated substrate (P) in the reaction solution were measured using LabChip EZ Reader II system (manufactured by Caliper Life Sciences) according to the protocol of the assay kit.

### (Method for Evaluating Inhibitory Activity)

The heights of the peaks of the “substrate” and the “phosphorylated substrate” were expressed as S and P, respectively, and a blank containing the assay buffer instead of the enzyme solution was also measured.

The inhibition rate (%) of the test compound was calculated according to the following equation:

$$Inhibition rate ( \% ) = ( 1 - ( C - A ) / ( B - A ) ) \times 100$$

wherein, A, B, and C represent P/(P+S) of the blank well, P/(P+S) of the control solution well, and P/(P+S) of the compound-containing well, respectively.

The IC₅₀ value was calculated via a regression analysis of the inhibition rate and the test compound concentration (logarithmic value).

### (Evaluation Result)

The inhibiting activities of representative compounds of the present invention are shown against DYRK1A, DYRK1B, DYRK2, and DYRK3 in Table 40. The kinase activity inhibitory effect was indicated with the mark *** at an IC₅₀ value of less than 0.01 μM; the mark ** at 0.01 μM or more and less than 0.1 μM; the mark * at 0.1 μM or more and less than 1 μM; and the mark—at 1 μM or more.

1. A compound represented by: 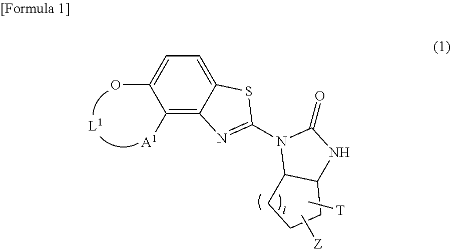
wherein
A¹ represents optionally substituted methylene or an oxygen atom,
L¹ represents optionally substituted methylene or optionally substituted ethylene,
1 represents 1, 2, or 3,
T represents a hydrogen atom or optionally substituted C₁₋₆ alkyl,
Z represents —NR¹R² or —OR³,
R¹ and R² each independently represent a hydrogen atom, optionally substituted C₁₋₆ alkyl, or
C(O)—R^{A}, or R¹ and R², together with the nitrogen atom to which they are attached, may form an optionally substituted 4- to 7-membered saturated heterocycle,
R^{A} represents —R^{A1} or —OR^{A1},
R^{A1} represents optionally substituted C₁₋₆ alkyl,
R³ represents a hydrogen atom, optionally substituted C₁₋₆ alkyl, or C(O)—R^{B}, and
R^{B} represents optionally substituted C₁₋₆ alkyl,
or a pharmaceutically acceptable salt thereof.

2. The compound according to claim 1 or a pharmaceutically acceptable salt thereof, wherein A¹ is methylene, and L¹ is optionally substituted methylene or optionally substituted ethylene.

3. The compound according to claim 1 or a pharmaceutically acceptable salt thereof, wherein A¹ is an oxygen atom, and L¹ is optionally substituted methylene or optionally substituted ethylene.

4. The compound according to claim 1 or a pharmaceutically acceptable salt thereof, wherein 1 is 1 or 2.

5. The compound according to claim 1 or a pharmaceutically acceptable salt thereof, wherein Z is —NR¹R².

6. The compound according to claim 1 or a pharmaceutically acceptable salt thereof, wherein Z is —OR³.

7. The compound according to claim 1 or a pharmaceutically acceptable salt thereof, wherein R¹ and R² each independently are a hydrogen atom or optionally substituted C₁₋₆ alkyl.

8. The compound according to claim 1 or a pharmaceutically acceptable salt thereof, wherein R¹ and R², together with the nitrogen atom to which they are attached, form an optionally substituted 4- to 7-membered saturated heterocycle.

9. The compound according to claim 1 or a pharmaceutically acceptable salt thereof, wherein R³ is a hydrogen atom or optionally substituted C₁₋₆ alkyl.

10. The compound according to claim 1 or a pharmaceutically acceptable salt thereof, wherein T is methyl optionally substituted with a halogen atom.

11. The compound according to claim 1 or a pharmaceutically acceptable salt thereof, wherein the compound is selected from the group of the following compounds:
(3aR,4R,6aR)-1-(7,8-dihydrofuro[3,2-e][1,3]benzothiazol 2-yl)-2-oxooctahydrocyclopenta[d]imidazol 4-yl rac-acetate (Example 6);
(3aR,4R,6aR)-3-(7,8-dihydrofuro[3,2-e][1,3]benzothiazol 2-yl)-2-oxooctahydrocyclopenta[d]imidazol 4-yl rac-acetate (Example 7);
rac-(3aR,6R,6aR)-1-(7,8-dihydrofuro[3,2-e][1,3]benzothiazol 2-yl)-6-hydroxyhexahydrocyclopenta[d]imidazol 2(1H)-one (Example 10);
rac-(3aR,4R,6aR)-1-(7,8-dihydrofuro[3,2-e][1,3]benzothiazol 2-yl)-4-hydroxyhexahydrocyclopenta[d]imidazol 2(1H)-one (Example 11);
rac-(3aR,4S,6aS)-1-(7,8-dihydrofuro[3,2-e][1,3]benzothiazol 2-yl)-4-(methylamino)hexahydrocyclopenta[d]imidazol 2(1H)-one (Example 14);
rac-(3aR,4S,6aS)-1-(7,8-dihydrofuro[3,2-e][1,3]benzothiazol 2-yl)-4-(dimethylamino)hexahydrocyclopenta[d]imidazol 2(1H)-one (Example 19);
rac-{[(3aR,4S,6aS)-1-(7,8-dihydrofuro[3,2-e][1,3]benzothiazol 2-yl)-2-oxooctahydrocyclopenta[d]imidazol 4-yl](methyl)amino}acetonitrile (Example 20);
rac-(3aR,6R,6aR)-1-(7,8-dihydrofuro[3,2-e][1,3]benzothiazol 2-yl)-6-(dimethylamino)hexahydrocyclopenta[d]imidazol 2(1H)-one (Example 21);
rac-{[(4R)-3-(7,8-dihydrofuro[3,2-e][1,3]benzothiazol 2-yl)-2-oxooctahydrocyclopenta[d]imidazol 4-yl](methyl)amino}acetonitrile (Example 22);
rac-{[(3aR,5R,6aS)-1-(7,8-dihydrofuro[3,2-e][1,3]benzothiazol 2-yl)-2-oxooctahydrocyclopenta[d]imidazol 5-yl](methyl)amino}acetonitrile (Example 30);
rac-{[(3aR,5R,6aS)-1-(7,8-dihydrofuro[3,2-e][1,3]benzothiazol 2-yl)-2-oxooctahydrocyclopenta[d]imidazol 5-yl]amino}acetonitrile (Example 31);
rac-(3aR,6R,6aR)-6-(3,3-difluoroazetidin-1-yl)-1-(7,8-dihydrofuro[3,2-e][1,3]benzothiazol 2-yl)hexahydrocyclopenta[d]imidazol 2(1H)-one (Example 34);
(3aS,4R,6aR)-4-amino-1-(7,8-dihydrofuro[3,2-e][1,3]benzothiazol 2-yl)hexahydrocyclopenta[d]imidazol 2(1H)-one (Example 37);
(3aS,4R,6aR)-4-(cyclopropylamino)-1-(7,8-dihydrofuro[3,2-e][1,3]benzothiazol-2-yl)hexahydrocyclopenta[d]imidazol 2(1H)-one (Example 40);
rac-(3aR,4S,6aS)-4-(3,3-difluoroazetidin-1-yl)-1-(7,8-dihydrofuro[3,2-e][1,3]benzothiazol-2-yl)hexahydrocyclopenta[d]imidazol-2(1H)-one (Example 45);
rac-(3aR,6R,6aS)-6-(cyclopropylamino)-1-(7,8-dihydrofuro[3,2-e][1,3]benzothiazol-2-yl)hexahydrocyclopenta[d]imidazol-2(1H)-one (Example 46);
rac-(3aR,6R,6aR)-1-(7,8-dihydrofuro[3,2-e][1,3]benzothiazol-2-yl)-6-(morpholin-4-yl)hexahydrocyclopenta[d]imidazol-2(1H)-one (Example 47);
rac-(3aR,6R,6aR)-6-[cyclopropyl(methyl)amino]-1-(7,8-dihydrofuro[3,2-e][1,3]benzothiazol-2-yl)hexahydrocyclopenta[d]imidazol-2(1H)-one (Example 49);
(3aS,6S,6aS)-1-(7,8-dihydrofuro[3,2-e][1,3]benzothiazol-2-yl)-6-(dimethylamino)hexahydrocyclopenta[d]imidazol-2(1H)-one (Example 50);
(3aR,4R,5S,6aR)-1-(7,8-dihydrofuro[3,2-e][1,3]benzothiazol-2-yl)-4-(dimethylamino)-5-methylhexahydrocyclopenta[d]imidazol-2(1H)-one (Example 57);
(3aR,4R,5S,6aR)-1-(7,8-dihydrofuro[3,2-e][1,3]benzothiazol-2-yl)-4-(dimethylamino)-5-(fluoromethyl)hexahydrocyclopenta[d]imidazol-2(1H)-one (Example 58);
(3aR,4R,5S,6aR)-4-(dimethylamino)-1-(2H-[1,3]dioxolo[4,5-e][1,3]benzothiazol-7-yl)-5-methylhexahydrocyclopenta[d]imidazol-2(1H)-one (Example 59); and
(3aR,4R,5S,6aR)-4-(dimethylamino)-1-(2H-[1,3]dioxolo[4,5-e][1,3]benzothiazol-7-yl)-5-(fluoromethyl)hexahydrocyclopenta[d]imidazol-2(1H)-one (Example 60).

12. A medicament comprising the compound according to claim 1 or a pharmaceutically acceptable salt thereof as an active ingredient.

13. A pharmaceutical composition comprising the compound according to claim 1 or a pharmaceutically acceptable salt thereof as an active ingredient.

14. A therapeutic agent and/or a prophylactic agent for a disease involving DYRK, comprising the compound according to claim 1 or a pharmaceutically acceptable salt thereof as an active ingredient.

15. The therapeutic agent and/or the prophylactic agent according to claim 14, wherein the disease involving DYRK is frontotemporal dementia, progressive supranuclear palsy, corticobasal degeneration, Lewy body dementia, vascular dementia, traumatic brain injury, chronic traumatic encephalopathy, stroke, Alzheimer's disease, Parkinson's disease, Down's syndrome, or depression, and mental retardation, memory impairment, memory loss, learning disability, intellectual disability, cognitive dysfunction, mild cognitive impairment, or dementia symptom associated therewith, or brain tumor, pancreatic cancer, ovarian cancer, osteosarcoma, colorectal cancer, lung cancer, bone resorption disease, osteoporosis, sickle cell anemia, chronic renal disease, or bone resorption disease.

16. A method for treating and/or preventing a disease involving DYRK, comprising administration of a therapeutically effective amount of the compound according to claim 1 or a pharmaceutically acceptable salt thereof to a patient in need of treatment.

17. Use of the compound according to claim 1 or a pharmaceutically acceptable salt thereof, for producing a therapeutic agent and/or a prophylactic agent for a disease involving DYRK.

18. The compound according to claim 1 or a pharmaceutically acceptable salt thereof, for use in treatment and/or prevention of a disease involving DYRK.

19. A medicament obtained by combining the medicament according to claim 12 and at least one or more agents selected from agents classified into an anticancer agent, an antipsychotic drug, an antidementia drug, an antiepileptic drug, an antidepressant drug, a gastrointestinal drug, a thyroid hormone drug, or an antithyroid drug.

20. The medicament according to claim 12, for treating frontotemporal dementia, progressive supranuclear palsy, corticobasal degeneration, Levy body dementia, vascular dementia, traumatic brain injury, chronic traumatic encephalopathy, stroke, Alzheimer's disease, Parkinson's disease, Down's syndrome, or depression, and complication, mental retardation, memory impairment, memory loss, learning disability, intellectual disability, cognitive dysfunction, mild cognitive impairment, or treating dementia symptom progression or preventing dementia onset associated therewith, or treating brain tumor, pancreatic cancer, ovarian cancer, osteosarcoma, colorectal cancer, lung cancer, bone resorption disease, osteoporosis, sickle cell anemia, chronic renal disease, or bone resorption disease, in combination with at least one or more agents selected from agents classified into an anticancer agent, an antipsychotic drug, an antidementia drug, an antiepileptic drug, an antidepressant drug, a gastrointestinal drug, a thyroid hormone drug, or an antithyroid drug.
